(19) United States
(12) Patent Application Publication
(10) Pub. No.: US 2024/0002380 A1
(43) Pub. Date: Jan. 4, 2024

(54) REV-ERB AGONISTS

(71) Applicants: Saint Louis University, St. Louis, MO (US); Pelagos Pharmaceuticals, Inc., Houston, TX (US)

(72) Inventors: John K. Walker, St. Louis, MO (US); Thomas Patrick Burris, Columbia, IL (US); Sadichha Sitaula, Clarksburg, MD (US); Arindam Chatterjee, Ballwin, MO (US); Napoleon Clement D'Cunha, St. Louis, MO (US); Eric Jon Jacobsen, Wildwood, MO (US); James Robert Blinn, O'Fallon, MO (US)

(73) Assignees: Saint Louis University, St. Louis, MO (US); Pelagos Pharmaceuticals, Inc., Houston, TX (US)

(21) Appl. No.: 18/134,055

(22) Filed: Apr. 13, 2023

Related U.S. Application Data
(63) Continuation of application No. PCT/US2021/055165, filed on Oct. 15, 2021.
(60) Provisional application No. 63/092,893, filed on Oct. 16, 2020.

(51) Int. Cl. C07D 471/04; C07D 487/04; C07D 215/227; A61K 9/00; A61P 25/28

(52) U.S. Cl. CPC C07D 471/04; C07D 487/04; A61P 25/28; A61K 9/0019; C07D 215/227

(57) ABSTRACT

Disclosed are compounds that regulate REV-ERB nuclear receptors that would be useful for treatment of various diseases. These compounds may be used in therapeutic applications such as in the treatment of neurodegenerative disease such as anxiety disorder, autoimmune diseases or disorders, or muscular disorders such as sarcopenia.

### CROSS-REFERENCE TO RELATED APPLICATIONS

This application is a Continuation of International Patent Application No. PCT/US2021/055165, entitled “REV-ERB AGONISTS” which was filed Oct. 15, 2021, the entire contents of which is incorporated herein by reference in its entirety. International Patent Application No. PCT/US2021/055165 claims priority to U.S. Provisional Patent Application No. 63/092,893, entitled “REV-ERB AGONISTS” which was filed Oct. 16, 2020, the entire contents of which is incorporated herein by reference in its entirety.

### GOVERNMENT SUPPORT CLAUSE

This invention was made with government support under Grant No. W81WH-16-1-0236 awarded by the United States Department of Defense. The government has certain rights in the invention.

### FIELD OF THE DISCLOSURE

The disclosure relates to the fields of pathology, nuclear receptors, molecular biology and pharmaceuticals. More specifically, the disclosure relates to agonists for the treatment of REV-ERB nuclear receptors that may be used in the treatment of one or more diseases or disorders. The compounds regulate REV-ERB nuclear receptors that would be useful for treatment of various diseases.

### BACKGROUND OF THE DISCLOSURE

Nuclear receptors are generally classified as ligand-regulated transcription factors since many of the members serve as receptors for a variety of physiological ligands including steroid hormones, lipids and fatty acids. The nuclear receptor superfamily is one of the primary classes of therapeutic drug targets for human disease. Members of the nuclear receptor family have a conserved modular domain structure. Binding of ligands to a region called the ligand-binding domain (LBD) causes a conformational change in this domain that results in a cascade of downstream events. A number of hormones, such as the steroid hormones (estrogens, progestins, glucocorticoids, androgens, and mineralocorticoids) and thyroid hormones, were identified well before they were known to target members of the nuclear receptor superfamily (before the existence of the superfamily was even known) and development of analogues of these ligands led to the design of many therapeutic compounds. Currently, physiological ligands are known for more than half of the nuclear receptor superfamily (of which there are 48 members in humans). The success of drugs that target ligand-regulated nuclear receptors led to substantial interest in identification of either natural or synthetic ligands for the “orphan” members of the superfamily that could be used as chemical tools to probe receptor function and to understand their potential therapeutic value.

The REV-ERB nuclear receptors were originally identified as orphan receptors. The REV-ERBs obtained their unusual name due to the unique genomic organization of REV-ERBα. REV-ERBβ is encoded by the opposite DNA strand of the c-erbA oncogene and hence the name is derived from “reverse strand of c-erbA”.

REV-ERBα and REV-ERBβ are orphan nuclear receptors (NRs) which are present in numerous tissues such as skeletal muscles, brain, adipose tissues, and the liver. These receptors along with ROR work to modulate inflammation in the body. The REV-ERBα was originally identified as an orphan NR based on its canonical NR domain structure. REV-ERBα was identified based on its homology to other NRs and has an overlapping expression pattern with REV-ERBα. Although considerably more is known about the function of REV-ERBα than REV-ERBO, this overlap in expression along with the similarity in DNA-binding and transcriptional activity indicates that they are likely to lack the carboxy-terminal tail of the LBD called activation function 2 (AF-2, helix 12), which is required for coactivator recognition. Instead, both of these receptors have been shown to be repressors of transcription due to their binding to corepressors such as NcoR. Both receptors also bind to identical DNA response elements termed RevREs where they were observed to constitutively repress target gene transcription via active recruitment of transcriptional corepressors. Recently, heme has been identified as an endogenous ligand for these NRs, but recent work has been on developing synthetic ligands for this target. While work has been done to develop these potential therapeutics, none have advanced to a clinical setting yet.

The discovery that the REV-ERBs are ligand-regulated as well as considerable information regarding the therapeutic potential of targeting the REV-ERBs led to the discovery of synthetic REV-ERB ligands and their validation in several models of human disease including type 2 diabetes, obesity, heart disease, autoimmunity, chronic inflammation, anxiety, sleep disorders, cancer, muscular dystrophy and cognitive disorders.

Given the importance of these NRs, the development of therapeutic agents that modulate the activity of REV-ERBα and REV-ERBβ, particular those that can target the NRs in the blood-brain barrier, is of commercial interest.

Thus, it is desirable to develop compounds that regulate REV-ERB nuclear receptors that would be useful for treatment of various diseases.

### SUMMARY OF THE INVENTION

An object of this invention is to provide compounds that regulate REV-ERB nuclear receptors.

Thus, in accordance with the present disclosure, there are provided compounds useful for modulating, such as antagonizing the activity of REV-ERB.

This disclosure provides a compound, or a pharmaceutically acceptable salt, hydrate or solvate thereof, of Formula I

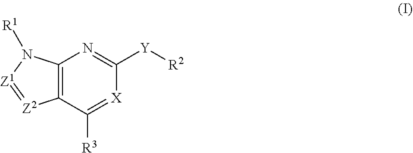

wherein
X is CR⁴ or N;
Y is a direct bond, O, S, SO, SO₂, CH₂ or NR⁵ or N⁺(R⁵)₂;
Z¹ is CR⁹ or N;
Z² is CR¹⁰ or N;
R¹ is C₁-C₅ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R¹¹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkylene-G¹, OC₁-C₄ alkylene-G¹ or C₃-C₇-cycloalkyl; or G²;
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
each R⁹ and R¹⁰ is independently H, C₁-C₆ alkyl, halogen, C₁-C₅ haloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R¹¹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R¹¹ is independently H, C₁-C₅ alkyl, C₁-C₅ haloalkyl, or C₁-C₅ alkyl substituted with NR⁵R⁵ or OR⁵.

This disclosure also provides a compound, or a pharmaceutically acceptable salt, hydrate or solvate thereof, of Formula II:

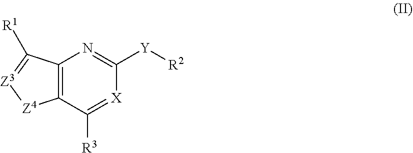

X is CR⁴ or N;
Y is a direct bond, O, S, SO, SO₂, CH₂ or NR⁵ or N⁺(R⁵)₂;
Z³ is CR⁹ or N;
Z⁴ is O, S or NR¹⁰;
R¹ is C₁-C₅ haloalkyl, halogen, cyano, C₁-C₅ alkoxy or NR⁵R⁵; or C₁-C₅ alkyl or C₃-C₇ cycloalkyl, each optionally substituted with one or more groups selected from halogen, hydroxy, C₁-C₅ alkoxy, or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R¹¹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or —OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or —OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇ cycloalkyl; or G².
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
R⁸ is G¹, O—C₁-C₄ alkylene-G³, NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R¹² on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R¹² on nitrogen atom ring members;
each R⁹ is independently H, C₁-C₆ alkyl, halogen, C₁-C₅ haloalkyl, cyano, C₁-C₅ alkoxy, or O—C₃-C₇ cycloalkyl;
each R¹⁰ is independently H, C₁-C₆ alkyl or C₁-C₅ haloalkyl;
R¹¹ is H, C₁-C₆ alkyl or C₁-C₅ haloalkyl; and
each R¹² is independently H, C₁-C₆ alkyl or C₁-C₅ haloalkyl.

This disclosure also provides a compound, or a pharmaceutically acceptable salt, hydrate or solvate thereof, of Formula III:

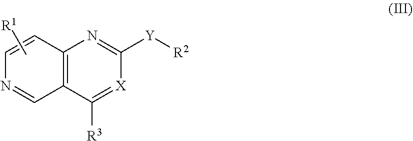

X is CR⁴ or N;
Y is a direct bond, O, S, SO, SO₂, CH₂ or NR⁵ or N⁺(R⁵)₂;
R¹ is H, halogen, NHR⁵, C₁-C₅ haloalkyl, C₁-C₅ alkoxy, or O—C₃-C₇ cycloalkyl; or C₁-C₆ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇-cycloalkyl; or G².
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
each R⁹ and R¹⁰ is independently H, C₁-C₆ alkyl, halogen, C₁-C₅ haloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R⁹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R⁹ is independently H, C₁-C₅ alkyl, C₁-C₅ haloalkyl, or C₁-C₅ alkyl substituted with NR⁵R⁵ or OR⁵.

This disclosure also provides a compound, or a pharmaceutically acceptable salt, hydrate or solvate thereof, of Formula IV

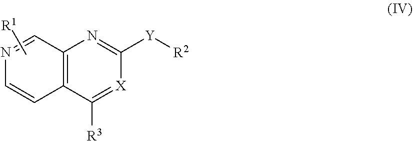

X is CR⁴ or N;
Y is a direct bond, O, S, SO, SO₂, CH₂ or NR⁵ or N⁺(R⁵)₂;
R¹ is H, halogen, NHR⁵, C₁-C₅ haloalkyl, C₁-C₅ alkoxy, or O—C₃-C₇ cycloalkyl; or C₁-C₆ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇-cycloalkyl; or G².
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
each R⁹ and R¹⁰ is independently H, C₁-C₆ alkyl, halogen, C₁-C₅ haloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R¹¹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R⁹ is independently H, C₁-C₅ alkyl, C₁-C₅ haloalkyl, or C₁-C₅ alkyl substituted with NR⁵R⁵ or OR⁵.

The disclosure also provides a compound, or a pharmaceutically acceptable salt, hydrate or solvate thereof, of Formula V

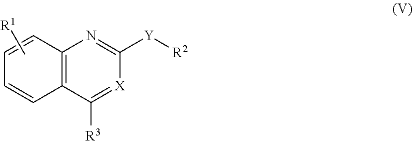

wherein
X is CR⁴ or N;
Y is a direct bond, O, S, SO, SO₂, CH₂ or NR⁵ or N⁺(R⁵)₂;
R¹ is H, halogen, NHR⁵, C₁-C₅ haloalkyl, C₁-C₅ alkoxy, or O—C₃-C₇ cycloalkyl; or C₁-C₆ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇ cycloalkyl; or G²;
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R⁹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R⁹ is independently H, C₁-C₅ alkyl, C₁-C₅ haloalkyl, or C₁-C₅ alkyl substituted with NR⁵R⁵ or OR⁵.

This disclosure also provides a compound, or a pharmaceutically acceptable salt, hydrate or solvate thereof, of Formula VI

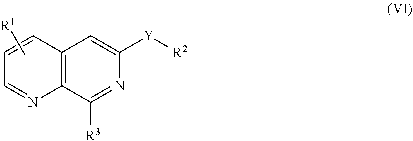

wherein
X is CR⁴ or N;
Y is a direct bond, O, S, SO, SO₂, CH₂ or NR⁵ or N⁺(R⁵)₂;
R¹ is H, halogen, NHR⁵, C₁-C₅ haloalkyl, C₁-C₅ alkoxy, or O—C₃-C₇ cycloalkyl; or C₁-C₆ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇ cycloalkyl; or G².
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R⁹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R⁹ is independently H, C₁-C₅ alkyl, C₁-C₅ haloalkyl, or C₁-C₅ alkyl substituted with NR⁵R⁵ or OR⁵.

This disclosure also provides a composition comprising a compound of Formula I, II, III, IV, V or VI as described above and a pharmaceutically acceptable adjuvant.

This disclosure also provides a method for treating a subject suffering from type 2 diabetes, obesity, heart disease such as congestive heart failure, autoimmunity and autoimmune diseases such as multiple sclerosis (MS) and rheumatoid arthritis, chronic inflammation and inflammatory diseases such as Non-Alcoholic SteatoHepatitis (NASH) and irritable bowel disease (IBD), neuroinflammation and neuroinflammatory diseases such as Alzheimer's disease and Parkinson's disease, sepsis such as caused by bacterial, viral or fungal infections, anxiety, sleep disorders, cancer, muscular dystrophy and cognitive disorders, the method comprising administering a pharmaceutically effective amount of a compound of Formula I, II, III, IV, V, VI, VII, or VIII as described above or a composition as described above.

Embodiments of this disclosure, including Embodiments of the Summary of the Disclosure or any other embodiments described herein, can be combined in any manner, and the descriptions of variables in the embodiments pertain not only to the compositions of this disclosure, but also to the methods or uses of any of the compositions of the disclosure.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWING

FIG. 1A is a graph illustrating the mean plasma concentration over time after intraperitoneal injection of the compound of Example 71 (SLUPP-1799) in female and male mice.

FIG. 1B is a graph illustrating the mean plasma concentration over time after intraperitoneal injection of the compound Example 78 (SLUPP-1657) in female and male mice.

### DETAILED DESCRIPTION OF THE DISCLOSURE

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a mixture, composition or method that comprises a list of elements is not necessarily limited to only those elements but may include other elements not expressly listed or inherent to such mixture, composition or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a mixture, composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one, one or more, or at least one, and the singular word form of the element or component also includes the plural unless the number is obviously meant to be singular.

Unless stated otherwise, all percentages, parts, ratios, etc., are by weight. When an amount, concentration, or other value or parameter is given as either a range, preferred range or a range defined as being from a list of lower limits or lower preferable values to a list of upper limits or upper preferable values, this is to be understood as specifically disclosing any or all ranges formed from any pair of any lower range limit or preferred value and any upper range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range. When the term “about” is used in describing a value or an end-point of a range, the disclosure includes the specific value or end-point referred to.

In the above recitations, the term “alkyl”, used either alone or in compound words such as “haloalkyl” includes straight-chain or branched alkyl, such as, methyl, ethyl, n-propyl, i-propyl, or the different butyl, pentyl or hexyl isomers. “Alkoxy” includes, for example, methoxy, ethoxy, n-propyloxy, isopropyloxy and the different butoxy, pentoxy and hexyloxy isomers. “Alkylamino”, “dialkylamino”, and the like, are defined analogously to the above examples. “Cycloalkyl” includes, for example, cyclopropyl, cyclobutyl, cyclopentyl and cyclohexyl. The term “cycloalkoxy” denotes cycloalkyl linked through an oxygen atom such as cyclopentyloxy and cyclohexyloxy. The terms “alkylene” or “alkylenyl” means a hydrocarbon group substituted with two groups, including straight-chain or branched alkylene, such as, methylene, ethylene, 1,2-propylene, 1,3-propylene.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms, which may be the same or different. Examples of “haloalkyl” or “alkyl substituted with halogen” include F₃C, ClCH₂, CF₃CH₂ and CF₃CCl₂.

When the attachment point between a substituent group (e.g. R¹) and the remainder of compound of Formulae I, II, III, IV, V, VI, VII, or VIII is illustrated as floating, it can be attached to any available carbon atom or nitrogen atom of the remainder of the compound by replacement of a hydrogen atom.

The term “optionally substituted” refers to moieties that are unsubstituted or have at least one non-hydrogen substituent that does not extinguish the biological activity possessed by the unsubstituted analog. As used herein, the following definitions shall apply unless otherwise indicated. The term “optionally substituted with” is used interchangeably with the phrase “unsubstituted or unsubstituted with” or with the term “(un)substituted.” Unless otherwise indicated, an optionally substituted moiety may have a substituent at any substitutable position of the moiety, and each substitution is independent of the other. The phrase “optionally substituted with up to n substituents” (wherein n is an integer) means that the moiety is unsubstituted or is substituted at any substitutable position in the moiety with a number of substituents ≤n. For example, when n is 5, the group may be substituted with 0, 1, 2, 3, 4 or 5 substituents. If the moiety has less than n substitutable positions, the amount of substituents is limited to the maximum of substitutable positions on the moiety.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxylsulfonyl” means —SO₂OH; “aminosulfonyl” means —SO₂NH₂ and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

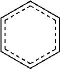

includes

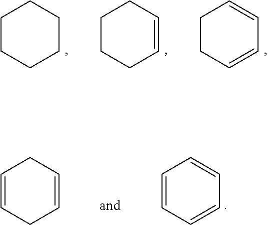

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

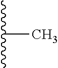

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

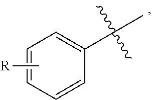

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

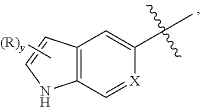

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and compound classes below, the number of carbon atoms in the group is as indicated as follows: “Cn” defines the exact number (n) of carbon atoms in the group/class. “C n” defines the maximum number (n) of carbon atoms that can be in the group/class, with the minimum number as small as possible for the group in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. Also compare “phosphine_{(C≤10)}”, which designates phosphine groups having from 0 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. Typically the carbon number indicator follows the group it modifies, is enclosed with parentheses, and is written entirely in subscript; however, the indicator may also precede the group, or be written without parentheses, without signifying any change in meaning. Thus, the terms “C5 olefin”, “C5-olefin”, “olefin_{(C5)}”, and “olefin_{C5}” are all synonymous. When any group or compound class below is used with the term “substituted”, any carbon atoms of the chemical group replacing the hydrogen atom do not count towards the total carbon atom limit for that group or compound class. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or an atom means the compound or atom has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

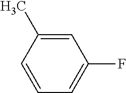

is also taken to refer to

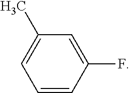

Aromatic compounds may also be depicted using a circle to represent the delocalized nature of the electrons in the fully conjugated cyclic π system, two non-limiting examples of which are shown below:

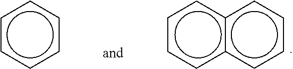

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound H—R, wherein R is alkyl as this term is defined above. When these terms are used with the “substituted” modifier one or more hydrogen atom has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

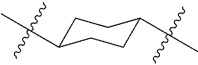 [SMILES: *C1CCC(*)CC1]

is a non-limiting example of cycloalkanediyl group. A “cycloalkane” refers to the compound H—R, wherein R is cycloalkyl as this term is defined above. When these terms are used with the “substituted” modifier one or more hydrogen atom has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When these terms are used with the “substituted” modifier one or more hydrogen atom has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When these terms are used with the “substituted” modifier one or more hydrogen atom has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

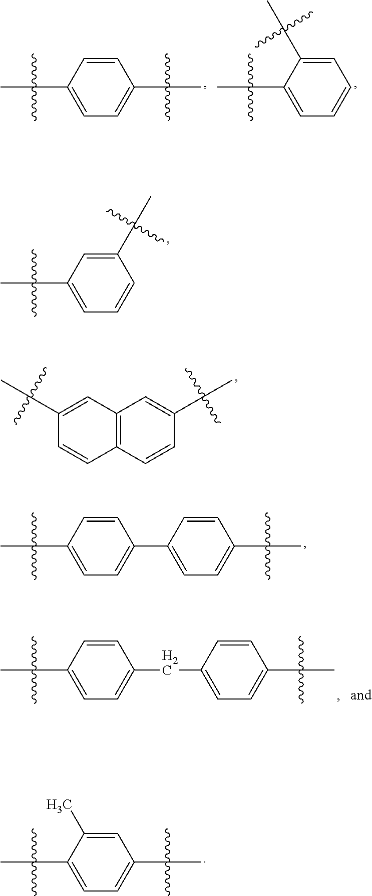

An “arene” refers to the compound H—R, wherein R is aryl as that term is defined above. Benzene and toluene are non-limiting examples of arenes. When these terms are used with the “substituted” modifier one or more hydrogen atom has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Furthermore, one or more of the sulfur atoms present in the group may be oxidized to the sulfonyl or sulfinyl state. If more than one ring is present, the rings may be fused or unfused in a pendent fashion. As used herein, the term does not preclude the presence of one or more alkyl, aryl, and/or aralkyl groups (carbon number limitation permitting) attached to the aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

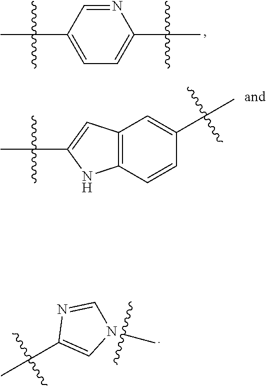

A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms or an aromatic group fused to the heterocycloalkyl group. Also, the term does not preclude the presence of one or more double bonds in the ring or ring system, provided that the resulting group contains at least one non-aromatic ring system which is the point of attachment. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group.

The term “acyl” when used without the “substituted” modifier refers to the group —C(O)R, in which R is a hydrogen, alkyl, cycloalkyl, alkenyl, aryl, aralkyl, heteroaryl, or heterocycloalkyl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —N₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —C(O)NHCH₃, —C(O)N(CH₃)₂, —OC(O)CH₃, —NHC(O)CH₃, —S(O)₂OH, —S(O)₂NH₂, or an amino protecting group.

The terms “alkylsulfinyl”, “alkylsulfinylamino”, “alkylsulfonyl”, and “alkylsulfonylamino” refers to the groups —S(O)R, —NHS(O)R, —S(O)₂R, and —NHS(O)₂R, respectively, in which R is an alkyl, as that term is defined above. The terms above may be used with any other appropriate chemical groups such as “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” wherein R is a cycloalkyl, alkenyl, alkynyl, aryl, aralkyl, heteroaryl, or heterocycloalkyl group, as those terms are defined above.

An “amino acid” is a functional group which contains a —CO₂H and a —NH₂ group on the same linear carbon skeleton. In its preferred embodiment, the term “amino acid” refers to one of the naturally occurring or commercially available amino acids as well as their enantiomers and diastereomers. As used herein, the term “amino acid residue” refers to a divalent amino acid which is linked through both the amine group and carboxylate group which are connected by an alkanediyl_{(C≤6)} which has been optionally substituted by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —OC(O)CH₃, —NHC(O)NH₂, —NHC(NH)NH₂, or —S(O)₂NH₂ or an alkyl_{(C1-12)}, alkenyl_{(C2-12)}, alkynyl_{(C2-12)}, aryl_{(C6-12)}, aralkyl_{(C7-12)}, heteroaryl_{(C1-12)}, heterocycloalkyl_{(C2-12)}, acyl_{(C1-12)}, or a substituted version of any of these groups wherein one or more hydrogen atoms on the chemical group has been substituted with —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —N(CH₂CH₃)₂, —C(O)NH₂, —OC(O)CH₃, —NHC(O)NH₂, —NHC(NH)NH₂, or —S(O)₂NH₂, e.g.

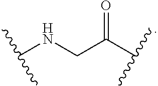 [SMILES: *NCC(*)=O]

In some embodiments, the amino acid residue is an α-amino acid wherein the alkanediyl is a methylene such that the carbonyl and the amine are joined by a single carbon. The amino acid residue may be one of the canonical amino acids such as leucine, isoleucine, tryptophan, cysteine, methionine, lysine, arginine, serine, threonine, tyrosine, phenylalanine, alanine, glycine, valine, glutamic acid, aspartic acid, asparagine, glutamine, proline, or histidine. These amino acid residues may be protected with one or more protecting groups on either the functional group on the side chain, the amine group, or the carboxylic acid group.

An “amino protecting group” is well understood in the art. An amino protecting group is a group which prevents the reactivity of the amine group during a reaction which modifies some other portion of the molecule and can be easily removed to generate the desired amine. Amino protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amino protecting group” can be a divalent protecting group such that both hydrogen atoms on a primary amine are replaced with a single protecting group. In such a situation the amino protecting group can be phthalimide (phth) or a substituted derivative thereof wherein the term “substituted” is as defined above. In some embodiments, the halogenated phthalimide derivative may be tetrachlorophthalimide (TCphth).

Throughout this application, the term “about” is used to indicate that a value includes the inherent variation of error for the device, the method being employed to determine the value, or the variation that exists among the study subjects, or +/−5% of the stated value.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living vertebrate organism, such as a human, monkey, cow, sheep, goat, dog, cat, mouse, rat, guinea pig, bird, fish or transgenic species thereof. In certain embodiments, the patient or subject is a primate. Non-limiting examples of human subjects are adults, juveniles, infants and fetuses.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of the compound of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease, including reactivation.

“Prodrug” means a compound that is convertible in vivo metabolically into an inhibitor according to the present disclosure. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-p-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diasteromers can be resolved or separated using techniques known in the art. It is contemplated that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Effective amount,” “therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease. In some embodiments, treatment of a patient afflicted with one of the pathological conditions described herein comprises administering to such a patient an amount of compound described herein which is therapeutically effective in controlling the condition or in prolonging the survivability of the patient beyond that expected in the absence of such treatment. As used herein, the term “inhibition” of the condition also refers to slowing, interrupting, arresting or stopping the condition and does not necessarily indicate a total elimination of the condition. It is believed that prolonging the survivability of a patient, beyond being a significant advantageous effect in and of itself, also indicates that the condition is beneficially controlled to some extent.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

Provided herein are compounds that may be used to modulate the activity of a nuclear receptor such as REV ERB. These compounds may be able to modulate the activity of either the REV ERBα or REV ERBO receptor. These compounds may be used to treat a disease or disorder associated with misregulation of these receptors and the biological pathways that these receptors regulate. The compounds may present a different scaffold of compounds than those known in the art or these compounds may show one or more favorable drug properties such as improved activity, pharmacokinetic profile, or stability. These details will be described in more detail.

The present disclosure relates to compounds of Formulae I, II, III, IV, V, VI, VII, and VIII, as defined in the Summary of the Disclosure, any of Preferred Embodiments A through S below and any other embodiments herein, compositions comprising the compounds, and methods for treating subjects suffering from type 2 diabetes, obesity, heart disease such as congestive heart failure, autoimmunity and autoimmune diseases such as multiple sclerosis (MS) and rheumatoid arthritis, chronic inflammation and inflammatory diseases such as Non-Alcoholic SteatoHepatitis (NASH) and irritable bowel disease (IBD), neuroinflammation and neuroinflammatory diseases such as Alzheimer's disease and Parkinson's disease, sepsis such as caused by bacterial, viral or fungal infections, anxiety, sleep disorders, cancer, muscular dystrophy and cognitive disorders comprising administering an effective amount of any of the compounds to the subject.

Notable indications for treating subjects include neuroinflammation, heart disease such as congestive heart failure, inflammatory diseases, sepsis and autoimmune diseases. More notable indications include neuroinflammation, sepsis and/or IBD.

Preferred Embodiments of compounds of Formula I include the following.

Preferred A: The compound of Formula I wherein
Y is O, S, SO, SO₂ or CH₂;
R¹ is C₁-C₅ alkyl;
R² is C₁-C₅-alkyl or C₃-C₆-cycloalkyl; or phenyl or naphthalenyl, each optionally substituted with up to 5 substituents independently selected from R⁶ and R⁷;
R³ is H or C₁-C₅ alkyl; and
R⁴ is H or C₁-C₅ alkyl.

Preferred B: The compound of Formula I wherein
Y is O;
Z¹ is CR⁹;
Z² is N;
R¹ is methyl;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R¹¹ on nitrogen atom ring members;
each R⁷ is independently H, C₁-C₆ alkyl, halogen, NHR⁵ or C₁-C₅ haloalkyl;
R⁹ is H or methyl; and
each R¹¹ is independently H or methyl.

Preferred C: The compound of Formula I wherein
Y is O;
Z¹ is N;
Z² is CR¹⁰;
R¹ is methyl;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R¹¹ on nitrogen atom ring members;
each R⁷ is independently H, C₁-C₆ alkyl, halogen, NHR⁵ or C₁-C₅ haloalkyl;
R¹⁰ is H or methyl; and
each R¹¹ is H or methyl.

Preferred Embodiments of compounds of Formula II include the following.

Preferred D: The compound of Formula II wherein
Y is O, S, SO, SO₂ or CH₂;
R¹ is C₁-C₅ alkyl;
R² is C₁-C₅-alkyl or C₃-C₆-cycloalkyl; or phenyl or naphthalenyl, each optionally substituted with up to 5 substituents independently selected from R⁶ and R⁷;
R³ is H or C₁-C₅ alkyl
R⁴ is H or C₁-C₅ alkyl;
each R⁹ is independently H, C₁-C₆ alkyl, halogen or C₁-C₅ haloalkyl;
each R¹⁰ is independently H, C₁-C₆ alkyl or C₁-C₅ haloalkyl;

Preferred E: The compound of Formula II wherein
Y is O;
Z³ is CR⁹;
Z⁴ is N;
R¹ is methyl;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R¹² on nitrogen atom ring members;
each R⁷ is independently H, C₁-C₆ alkyl, halogen, NHR⁵ or C₁-C₅ haloalkyl;
R⁹ is H or methyl;
R¹¹ is H or methyl; and
each R¹² is independently H or methyl.

Preferred F: The compound of Formula II wherein
Y is O;
Z¹ is N;
Z² is CR¹⁰;
R¹ is methyl;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R¹² on nitrogen atom ring members;
each R⁷ is independently selected from H, C₁-C₆ alkyl, halogen, NHR⁵ or C₁-C₅ haloalkyl;
R¹⁰ is H or methyl;
R¹¹ is H or methyl; and
each R¹² is independently H or methyl.

Preferred Embodiments of compounds of Formula III include the following.

Preferred G: The compound of Formula III wherein
Y is O, S, SO, SO₂, NH or N⁺(CH₃)₂;
R¹ is H or C₁-C₅ alkyl;
R² is C₁-C₅-alkyl or C₃-C₆-cycloalkyl; or phenyl or naphthalenyl, each optionally substituted with up to 5 substituents independently selected from R⁶ and R⁷;
R³ is H or C₁-C₅ alkyl; and
R⁴ is H or C₁-C₅ alkyl.

Preferred H: The compound of Formula III wherein
Y is O;
R¹ is H;
R³ is H; and
R⁴ is H.

Preferred J: The compound of Preferred H wherein
Y is O;
R¹ is H;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R⁹ on nitrogen atom ring members;
each R⁷ is independently H, C₁-C₆ alkyl, halogen, NHR⁵ or C₁-C₅ haloalkyl; and
each R⁹ is independently H or methyl.

Preferred Embodiments of compounds of Formula IV include the following.

Preferred K: The compound of Formula IV wherein
Y is O, S, SO, SO₂, NH or N⁺(CH₃)₂;
R¹ is H or C₁-C₅ alkyl;
R² is C₁-C₅-alkyl or C₃-C₆-cycloalkyl; or phenyl or naphthalenyl, each optionally substituted with up to 5 substituents independently selected from R⁶ and R⁷;
R³ is H or C₁-C₅ alkyl; and
R⁴ is H or C₁-C₅ alkyl.

Preferred L: The compound of Preferred K wherein
Y is O;
R¹ is H;
R³ is H; and
R⁴ is H.

Preferred M: The compound of Formula IV wherein
Y is O;
R¹ is H;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R⁹ on nitrogen atom ring members;
each R⁷ is independently H, C₁-C₆ alkyl, halogen, NHR⁵ or C₁-C₅ haloalkyl; and
each R⁹ is independently H or methyl.

Preferred Embodiments of compounds of Formula V include the following.

Preferred N: The compound of Formula V wherein
Y is O, S, SO, SO₂, NH or N⁺(CH₃)₂;
R¹ is H or C₁-C₅ alkyl;
R² is C₁-C₅-alkyl or C₃-C₆-cycloalkyl; or phenyl or naphthalenyl, each optionally substituted with up to 5 substituents independently selected from R⁶ and R⁷;
R³ is H or C₁-C₅ alkyl; and
R⁴ is H or C₁-C₅ alkyl.

Preferred O: The compound of Preferred N wherein
Y is O;
R¹ is H;
R³ is H; and
R⁴ is H.

Preferred P: The compound of Formula V wherein
Y is O;
R¹ is H;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R⁹ on nitrogen atom ring members;
each R⁷ is independently H, C₁-C₆ alkyl, halogen, NHR⁵ or C₁-C₅ haloalkyl; and
each R⁹ is independently H or methyl.

Preferred Embodiments of compounds of Formula VI include the following.

Preferred Q: The compound of Formula VI wherein
Y is O, S, SO, or SO₂;
R¹ is H or C₁-C₅ alkyl;
R² is C₁-C₅-alkyl or C₃-C₆-cycloalkyl; or phenyl or naphthalenyl, each optionally substituted with up to 5 substituents independently selected from R⁶ and R⁷;
R³ is H or C₁-C₅ alkyl; and
R⁴ is H or C₁-C₅ alkyl.

Preferred R: The compound of Preferred Q wherein
Y is O;
R¹ is H;
R³ is H; and
R⁴ is H.

Preferred S: The compound of Formula VI wherein
Y is O;
R¹ is H;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R⁹ on nitrogen atom ring members;
each R⁷ is independently H, C₁-C₆ alkyl, halogen, NHR⁵ or C₁-C₅ haloalkyl; and
each R⁹ is independently H or methyl.

The compounds of Formula of Formula I, II, III, IV, V, VI, VII, and VIII, including any embodiments thereof, can be prepared by general methods known in the art of synthetic organic chemistry.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

One or more of the following methods and variations as described in Schemes 1 through 6 can be used to prepare the compounds using methods illustrated in the general synthetic schemes and experimental procedures detailed below. The definitions of variables in the intermediates in the schemes are as defined above in the Summary of the Disclosure unless otherwise noted.

General synthetic schemes and experimental procedures are presented for purposes of illustration and are not intended to be limiting. Starting materials used to prepare compounds of the present disclosure are commercially available or can be prepared using routine methods known in the art. Solvents and reagents, whose synthetic preparations are not described below, can be purchased, for example at Sigma-Aldrich or Fisher Scientific.

### Preparation of 5-aryloxy-3H-imidazol[4,5-b]pyridines and 2-aryloxy-9H-purines

Scheme 1 highlights the general synthesis of 5-aryloxy-3H-imidazol[4,5-b]pyridines and 2-aryloxy-9H-purines. Alkylation of the imidazolpyridine or purine 1a with the desired R¹ substituent is carried out using sodium hydride and the appropriate halo-R¹ reagent in a solvent such as DMF or THF to give 1b. Displacement of the chloro group in 1b is accomplished by reaction with the desired alcohol (or amine or thioether) in the presence of a weak base such as potassium carbonate in a solvent such as DMF or THF to give 1c.

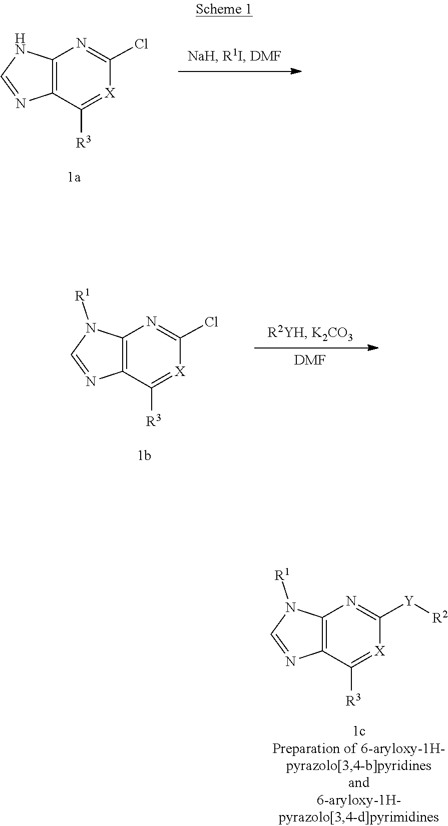

Scheme 2 highlights the general synthesis of 6-aryloxy-1H-pyrazolo[3,4-b]pyridines and 6-aryloxy-1H-pyrazolo[3,4-d]pyrimidines. Alkylation of the pyrazolopyridine or pyrazolopyrimidine 2a with the desired R¹ substituent is carried out using sodium hydride and the appropriate halo-R¹ reagent in a solvent such as DMF or THF to give 2b. Displacement of the chloro group in 2b is accomplished by reaction with the desired alcohol (or amine or thioether) in the presence of a weak base such as potassium carbonate in a solvent such as DMF or THF to give 2c.

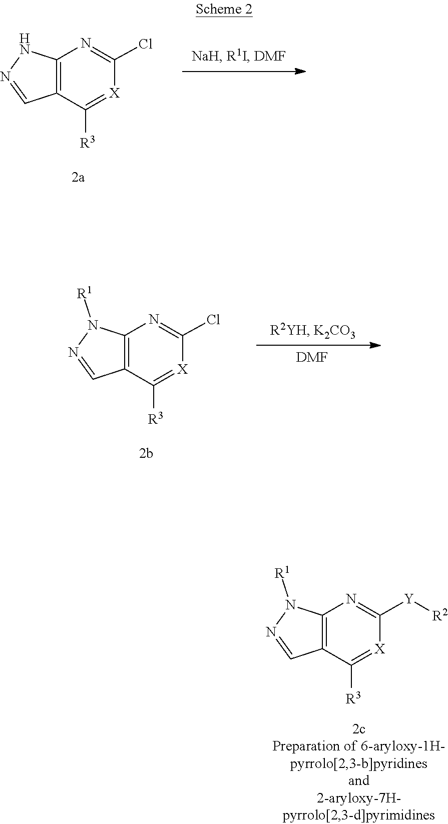

Scheme 3 highlights the general synthesis of 6-aryloxy-1H-pyrrolo[2,3-b]pyridines and 2-aryloxy-7H-pyrrolo[2,3-d]pyrimidines. Alkylation of the pyrrolopyridine or pyrrolopyrimidine 3a with the desired R¹ substituent is carried out using sodium hydride and the appropriate halo-R¹ reagent in a solvent such as DMF or THF to give 3b. Displacement of the chloro group in 3b is accomplished by reaction with the desired alcohol (or amine or thioether) in the presence of a weak base such as potassium carbonate in a solvent such as DMF or THF to give 3c.

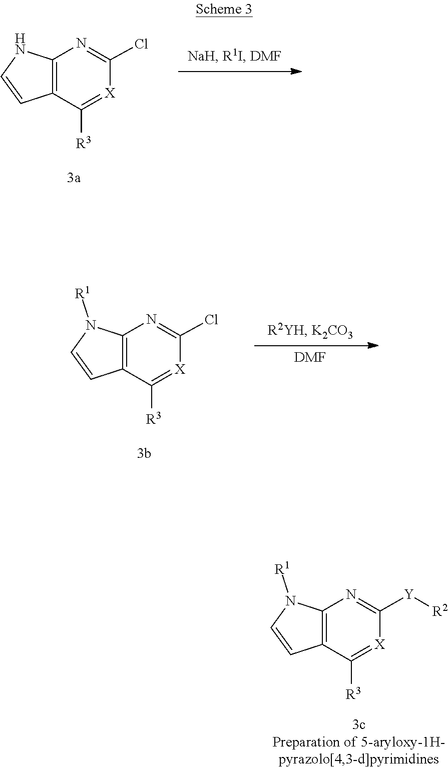

Scheme 4 highlights the general synthesis of aryloxy-pyrazolo[4,3-d]pyrimidines. Exposure of pyrazole 4a to a mixture of nitric acid and sulfuric acid provides nitro analog 4b. Methylation of the acid of 4b is accomplished using iodomethane and potassium carbonate in DMF to give ester 4c. Reduction of the nitro group of 4c is accomplished using Raney nickel in methanol to give amine 4d. Reaction of 4d with benzoyl isothiocyanate in acetone provides a thiourea intermediate which was cyclized in a water/acetone mixture in the presence of potassium carbonate to give thioxo pyrazolopyrimidine-one 4e. Thiourea 4e was alkylated using iodomethane and potassium carbonate in a solvent such as DMF to give thioether 4f. Exposure of amide 4f to phosphorous oxychloride and pyridine in DMF provides chloro 4g. Hydrogenolysis of 4g using palladium on carbon as the catalyst in isopropanol provides 4h where R³ is hydrogen. If desired, 4g may be converted to analogs with a R³ substituent using methods known to those skilled in the art. Oxidation of thioether 4h with meta-chloroperbenzoic acid in dichloromethane gives sulfone 4i. Displacement of the sulfone group in 4i is accomplished by reaction with the desired alcohol (or amine or thioether) in the presence of a weak base such as potassium carbonate in a solvent such as DMF or THF to give 4j.

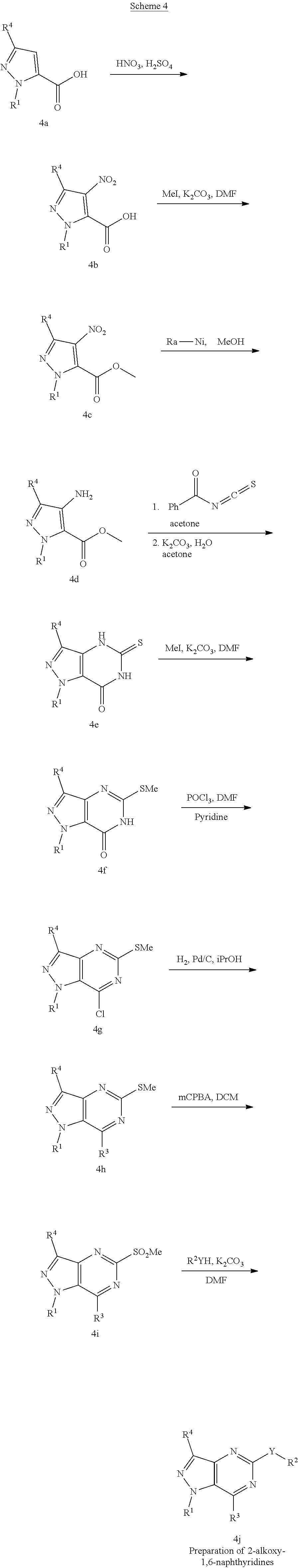

Scheme 5 highlights the general synthesis of alkoxy-naphthyridines and alkoxy-quinolines. Displacement of the chloro group in 5a is accomplished by reaction with the desired alcohol (or amine or thioether) in the presence of a weak base such as potassium carbonate in a solvent such as DMF or THF to give 5b.

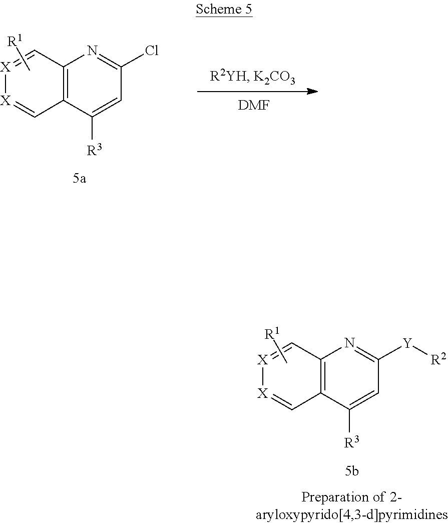

Scheme 6 highlights the general synthesis of 2-aryloxypyrido[4,3-d]pyrimidines. Reaction of ethyl 3-oxobutanoate with acetic anhydride and triethoxymethane gives enol ether 6b. Exposure of 6b to 2-methyl-2-thiopseudourea sulfate and trimethylamine as a weak base in ethanol provides cyclized product 6c. Formation of the enamine is carried out by treating 6c with 1,1-dimethyl-N,N-dimethylmethanamine in DMF as a solvent gives 6d. Reaction of 6d with ammonium acetate in a mixture of ammonium hydroxide and ethanol to give pyridopyrimidone 6e. Exposure of amide 6e to phosphorous oxychloride provides chloro 6f. Hydrogenolysis of 6f using palladium on carbon as the catalyst with ammonium formate in methanol provides 6g. Reaction of 6g with sulfuryl chloride in a dichloromethane/acetonitrile solvent mixture gives a crude intermediate which was reacted with the desired alcohol (or amine or thioether) in the presence of a weak base such as potassium carbonate in a solvent such as DMF to give 6h.

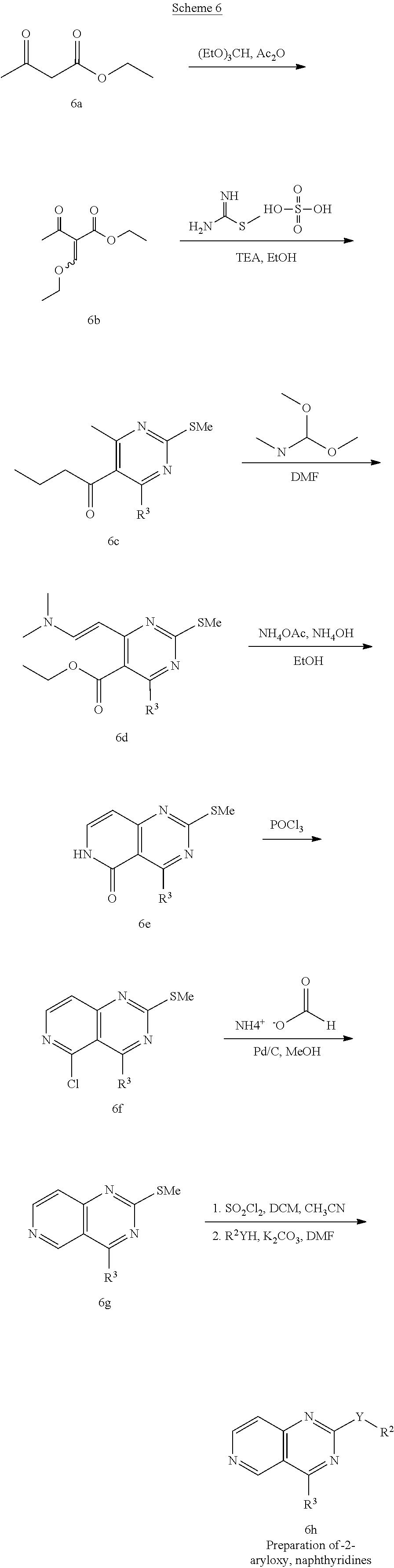

Scheme 7 highlights the general synthesis of aryloxy-naphthyridines and aryloxy-quinolines. Displacement of the chloro group in 7a is accomplished by addition of the appropriately substituted phenoxy compound in the presence of a weak base such as potassium carbonate in a solvent such as DMF to give 7c. Alternatively, the chloro can be displaced in with the appropriately substituted 2-bromophenol to give compound 7b. The corresponding aryl bromide can undergo a palladium catalyzed cross-coupling with the desired aryl bromide using a catalyst such tetrakis(triphenylphosphine)palladium(0), a weak base such as potassium carbonate in a solvent such as dioxane to give 7c.

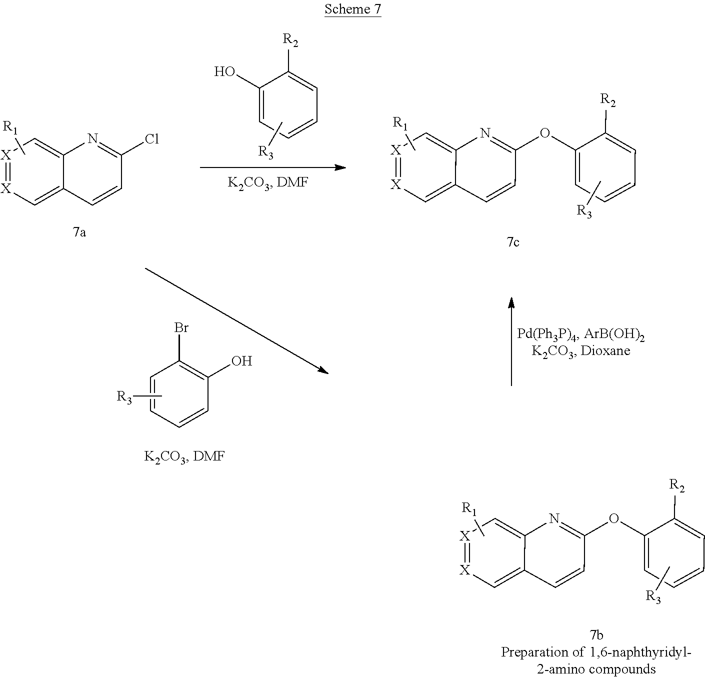

Scheme 8 highlights the general synthesis for 2-aminosubstituted 1,6-naphthyridinmes. The corresponding chloro group in the starting material, 8a, can be replaced with an amino group via a palladium catalyzed amine Buchwald type reaction, using a palladium source such as Tris(dibenzylidineacetone)palladium(0), a catalyst such as BINAP, an alkoxy base such as sodium tbutoxide in a solvent such as toluene to give 8b. Methylation of the corresponding amine group can be achieved through reaction of 8b with iodomethane in a solvent such as acetone to give compound 8c.

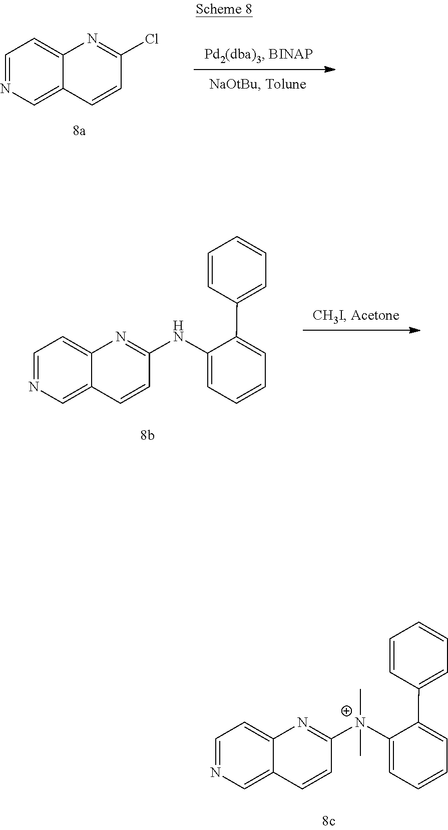 [SMILES: Clc1ccc2cnccc2n1 ; c1ccc(-c2ccccc2Nc2ccc3cnccc3n2)cc1 ; C[N+](C)(c1ccc2cnccc2n1)c1ccccc1-c1ccccc1]

It is recognized by one skilled in the art that various functional groups can be converted into others to provide different compounds of Formulae I, II, III, IV, V and VI. Compounds of Formulae I, II, III, IV, V and VI and the intermediates described herein can be subjected to various electrophilic, nucleophilic, radical, organometallic, oxidation, and reduction reactions to add substituents or modify existing substituents. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. The above reactions can also in many cases be performed in alternate order.

Any of the compounds of any of Formulae I, II, III, IV, V and VI may be obtained as the free base or a pharmaceutically acceptable salt. One skilled in the art recognizes that because under physiological conditions salts of chemical compounds are in equilibrium with their corresponding nonsalt forms, salts share the biological utility of the nonsalt forms. Thus a wide variety of salts of a compound any of Formulae I, II, III, IV, V and VI are useful for treating subjects suffereing from disease according to this disclosure (i.e. are pharmaceutically acceptable). The salts of a compound of any of Formulae I, II, III, IV, V and VI include acid-addition salts with inorganic or organic acids such as hydrobromic, hydrochloric, nitric, phosphoric, sulfuric, acetic, trifluroacetic, butyric, fumaric, lactic, maleic, malonic, oxalic, propionic, salicylic, tartaric, 4-toluenesulfonic or valeric acids. When a compound of any of Formulae I, II, III, IV, V and VI contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. When a compound of any of Formulae I, II, III, IV, V and VI comprises a quaternary ammonium ion (e.g. Y is N⁺(R⁵)₂), salts also include halides such as iodides, hydroxides or carbonates. Accordingly, the present disclosure comprises compounds of any of Formulae I, II, III, IV, V and VI and pharmaceutically acceptable salts thereof. Notably, trifluoracetic acid salts of compounds of Formulae I, II, III, IV, V and VI may be obtained after removal of Boc protecting groups. Notably, iodide salts of compounds of Formulae I, II, III, IV, V and VI may be obtained after alkylation of amino groups.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular order presented to prepare the compounds of the disclosure.

The compounds described herein are useful for regulating REV-ERBs and, consequently, may be used to treat several human disease including type 2 diabetes, obesity, heart disease, autoimmunity, chronic inflammation, anxiety, sleep disorders, cancer, muscular dystrophy and cognitive disorders.

### A. Therapeutic Activity

Nuclear receptors are a class of proteins which are prevalent in a wide variety of therapeutic applications. Nuclear receptors are proteins found with cells that are responsible for sensing steroid and thyroid hormones and other signaling molecules. These receptors often work with other proteins to regulate the expression of specific genes. The nuclear receptors often will bind directly to DNA and regulate the expression of genes. This binding process is generally controlled by the binding of a ligand. While there are many nuclear receptors, these proteins are generally grouped as thyroid hormone receptor-like, retinoid X receptor-like, estrogen receptor-like, nerve growth factor IB-like, steroidogenic factor-like, germ cell nuclear factor-like, nuclear receptor 8, nuclear receptors with two DNA binding domains, or miscellaneous nuclear receptors. The compounds described herein may be used to modulate the activity of one or more nuclear receptors such as the REV ERBα or REV ERBβ receptor. The compounds may disrupt the activity of these nuclear receptors. The disruption of these activities may be useful in one or more therapeutic applications such as neurodegenerative diseases, autoimmune disorders, or muscular disorders such as sarcopenia.

1. Neurodegenerative Diseases

In some embodiments, the compounds and methods described herein may be used to treat one or more neurodegenerative disease. A neurodegenerative disease, generally, refers to a disease or condition in which the function of a subject's nervous system becomes impaired. The term “neurodegenerative disease or disorder” and “neurological disorders” encompass a disease or disorder in which the peripheral nervous system or the central nervous system is principally involved. As used herein, the terms “neurodegenerative disease”, “neurodegenerative disorder”, “neurological disease”, and “neurological disorder” are used interchangeably.

Examples of neurological disorders or diseases include, but are not limited to chronic neurological diseases such as diabetic peripheral neuropathy (including third nerve palsy, mononeuropathy, mononeuropathy multiplex, diabetic amyotrophy, autonomic neuropathy and thoracoabdominal neuropathy), Alzheimer's disease, age-related memory loss, senility, age-related dementia, Pick's disease, diffuse Lewy body disease, progressive supranuclear palsy (Steel-Richardson syndrome), multisystem degeneration (Shy-Drager syndrome), motor neuron diseases including amyotrophic lateral sclerosis (“ALS”), degenerative ataxias, cortical basal degeneration, ALS-Parkinson's-Dementia complex of Guam, subacute sclerosing panencephalitis, Huntington's disease, Parkinson's disease, multiple sclerosis (“MS”), synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Wernicke-Korsakoffs related dementia (alcohol induced dementia), Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia). Other conditions also included within the methods of the present invention include age-related dementia and other dementias, and conditions with memory loss including vascular dementia, diffuse white matter disease (Binswanger's disease), dementia of endocrine or metabolic origin, dementia of head trauma and diffuse brain damage, dementia pugilistica, and frontal lobe dementia. Also other neurodegenerative disorders resulting from cerebral ischemia or infarction including embolic occlusion and thrombotic occlusion as well as intracranial hemorrhage of any type (including, but not limited to, epidural, subdural, subarachnoid, and intracerebral), and intracranial and intravertebral lesions (including, but not limited to, contusion, penetration, shear, compression, and laceration). Thus, the term also encompasses acute neurodegenerative disorders such as those involving stroke, traumatic brain injury, schizophrenia, peripheral nerve damage, hypoglycemia, spinal cord injury, epilepsy, and anoxia and hypoxia.

In some embodiments, the neurodegenerative disorder is amyloidosis. Amyloidosis is observed in Alzheimer's Disease, hereditary cerebral angiopathy, nonneuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis arthropathy, and Finnish and Iowa amyloidosis.

Examples of neurodegenerative diseases that may be treated with a compound or method described herein include Parkinson's disease and analogous conditions such as drug-induced Parkinsonism, progressive supranuclear palsy, Idiopathic Parkinson's disease, Autosomal dominant Parkinson disease, Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or Mitochondrial Parkinson's disease.

(a) i. Fear and Anxiety Related Diseases and Disorders

Additionally, the neurodegenerative disorder may be a disorder or diseases associated with fear or anxiety. Fear and anxiety related diseases and disorders are associated with the dysregulation of the fear processing centers in the brain. In particular, testosterone or derivatives thereof or the formulations of the present disclosure may be used to treat a fear or anxiety related disease or disorder. Without being bound by theory, the treatment of these diseases and disorder with an agent that modulates the brain's response to fear is effective in treating these diseases and disorders. In general, phobias such as social phobias and non-social phobias are centered around the fear of a particular thing. In a non-limiting example, non-social phobias include arachnophobia, hemophobia, or chemophobia and related to a fear of specific object such as spiders, blood, and chemicals, respectively. Social phobia, on the other hand, is a fear of either a generalized or specific social situation. In a few non-limiting examples, social phobias can be associated with such generalized social situations as attending an event with a crowd, conversing with strangers, or meeting new people at a club. On the other hand, specific social phobias can include fear of public speaking, fear of conversing with a particular group such as the opposite gender, or a fear or interacting with a specific group of people such as dentist or doctors in a few non-limiting examples.

Furthermore, fear or anxiety related diseases or disorders include panic disorders which are associated with fear of a particular situation or stimulus that is present during an initial attack. Panic disorders are noted by the rapid and repeated onset of fear, in some cases, debilitating fear, which can impact an individual's ability to work and can last anywhere from minutes to hours. Additionally, the patient tends to be afraid of having another attack. Treatment of these diseases or disorders with compounds that can modulate the fear are potentially therapeutically important treatment options. Additionally, generalized anxiety disorder is when a patient exhibits anxiety towards a routine worry which cannot be resolved even when the patient no longer has a rational reason to worry.

Additionally, people can become fixed on patterns and routines such that these become an obsession. When the individual feels compelled to perform these activities as a means of reducing anxiety—even though the activities interfere with the individual's daily life—the individual may be diagnosed with obsessive compulsive disorder. Such anxiety-driven compulsions in an individual can be modulated by changing the fear response of the individual.

Finally, post-traumatic stress disorder (PTSD) results when the body's fight or flight systems become dysregulated from exposure to actual or imagined fearful stimuli. In individuals with PTSD, the individual continues to react as if the fearful stimuli are present even after the stimuli are removed. Traditionally, the disorder is most associated with war veterans but can occur after the individual experiences any traumatic event or someone close to the individual experiences a traumatic event. Often, these events are associated with a threat of bodily harm.

2. Auto-Immune Disorders or Inflammatory Conditions

As provided herein, the compounds may be used to modulate a REV-ERB NR such that it may be used to treat one or more autoimmune disorders or a condition associated with chronic inflammation. Autoimmune diseases are conditions that arise from an abnormal immune response to a functioning body part or organ system. As of now, there are 80 types of autoimmune disease. These autoimmune diseases often have a few minor symptoms in common that include low grade fever and a general feeling of lethargy or tired. The other symptoms vary with the specific autoimmune diseases. The causes of autoimmune disorders are generally unknown but some are known to run in families or have a genetic component. Others are triggered by an infection or some environmental factor. Current treatments vary with the type and severity of the conditions but often include some anti-inflammatory compounds such as an NSAID and/or an immunosuppressant. It is estimated that 24 million people in the US suffer from some form of autoimmune disease or disorder. Autoimmune disorders generally start during adulthood but can in some cases affect children. Furthermore, autoimmune disorders are more prevalent in women than in men.

Some non-limiting examples of such diseases or disorders include acquired immunodeficiency syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma, systemic scleroderma, progressive systemic sclerosis (PSS), systemic sclerosis (SS), Sjogren's syndrome, stiff-man syndrome, systemic lupus erythematosus (SLE), Takayasu arteritis, temporal arteritis/giant cell arteritis, inflammatory bowel disease (IBD), ulcerative colitis, Cohn's disease, intestinal mucosal inflammation, wasting disease associated with colitis, uveitis, vitiligo and Wegener's granulomatosis, Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy, ventilator induced lung injury, viral infections, autoimmune diabetes and the like. Inflammatory disorders include, for example, chronic and acute inflammatory disorders.

3. Sarcopenia

Sarcopenia (from the Greek meaning “poverty of flesh”) is the degenerative loss of skeletal muscle mass (0.5-1% loss per year after the age of 25), quality, and strength associated with aging. Sarcopenia is a component of the frailty syndrome. As of 2009, there was no generally accepted definition of sarcopenia in the medical literature.

Sarcopenia is characterized first by a muscle atrophy (a decrease in the size of the muscle), along with a reduction in muscle tissue “quality,” caused by such factors as replacement of muscle fibres with fat, an increase in fibrosis, changes in muscle metabolism, oxidative stress, and degeneration of the neuromuscular junction. Combined, these changes lead to progressive loss of muscle function and frailty.

Lack of exercise is currently thought to be a significant risk factor for sarcopenia. Not only muscle but the entire musculoskeletal system of muscle, neuromuscular responsiveness, endocrine function, vasocapillary access, tendon, joint, ligament, and bone, depends on regular and lifelong exercise to maintain integrity. Exercise and increases in activity have been shown to be beneficial in settings of sarcopenia, even in the very old. However, even highly trained athletes experience the effects of sarcopenia. Even Master class athletes who continue to train and compete throughout their adult life, exhibit a progressive loss of muscle mass and strength, and records in speed and strength events decline progressively after age 30.

Simple circumference measurement does not provide enough data to determine whether or not an individual is suffering from severe sarcopenia. Sarcopenia is also marked by a decrease in the circumference of distinct types of muscle fibers. Skeletal muscle has different fiber-types, which are characterized by expression of distinct myosin variants. During sarcopenia, there is a decrease in “type 2” fiber circumference (Type II), with little to no decrease in “type I” fiber circumference (Type I), and deinervated type 2 fibers are often converted to type 1 fibers by reinnervation by slow type 1 fiber motor nerves.

Satellite cells are small mononuclear cells that abut the muscle fiber. Satellite cells are normally activated upon injury or exercise. These cells then differentiate and fuse into the muscle fiber, helping to maintain its function. One theory is that sarcopenia is in part caused by a failure in satellite cell activation. Therefore, the ability to repair damaged muscles or respond to nutritional signals is impaired.

Extreme muscle loss is often a result of both diminishing anabolic signals, such as growth hormone and testosterone, and promotion of catabolic signals, such as proinflammatory cytokines.

Due to the lessened physical activity and increased longevity of industrialized populations, sarcopenia is emerging as a major health concern. Sarcopenia may progress to the extent that an older person may lose his or her ability to live independently. Furthermore, sarcopenia is an important independent predictor of disability in population-based studies, linked to poor balance, gait speed, falls, and fractures. Sarcopenia can be thought of as a muscular analog of osteoporosis, which is loss of bone, also caused by inactivity and counteracted by exercise. The combination of osteoporosis and sarcopenia results in the significant frailty often seen in the elderly population.

Exercise has been considered of great interest in treatment of sarcopenia. There are several reports showing increased ability and capacity of skeletal muscle to synthesize proteins in response to short term resistance exercise. Also, it has been reported exercise can improve physical performance (strength and mobility) in elderly subjects. However, there is insufficient research demonstrating such findings in long term.

Currently, there are no agents approved for treatment of sarcopenia. Possible therapeutic strategies include use of testosterone or anabolic steroids, though long term use of these agents is controversial in men given concerns of prostate symptoms, and essentially contraindicated in women, given concerns of virilization. Recent experimental results have shown testosterone treatments may induce adverse cardiovascular events. Other approved medications have been shown to have little to no effect in this setting, including agents such DHEA and human growth hormone. New therapies in clinical development hold great promise in this area, including the selective androgen receptor modulators (SARMs), as evidenced by recent studies. Nonsteriodal SARMs are of particular interest, given they exhibit significant selectivity between the anabolic effects of testosterone on muscle, but apparently with little to no androgenic effects such as prostate stimulation in men or virilization in women.

### C. Therapeutic Methods

1. Pharmaceutical Formulations

In particular embodiments, where clinical application of an active ingredient is undertaken, it will be necessary to prepare a pharmaceutical composition appropriate for the intended application. Generally, this will entail preparing a pharmaceutical composition that is essentially free of pyrogens, as well as any other impurities or contaminants that could be harmful to humans or animals. One also will generally desire to employ appropriate buffers to render the complex stable and allow for uptake by target cells.

Aqueous compositions of the present disclosure comprise an effective amount of the active compound, as discussed above, further dispersed in pharmaceutically acceptable carrier or aqueous medium. Such compositions also are referred to as inocula. The phrase “pharmaceutically or pharmacologically acceptable” refers to compositions that do not produce an adverse, allergic or other untoward reaction when administered to an animal, or a human, as appropriate, as well as the requisite sterility for in vivo uses.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the compositions.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The therapeutic compositions of the present disclosure are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, a controlled release patch, salve or spray. In some embodiments, the topical formulation by used for administration to the skin, to mucosa membranes such as the eye, the eye lids, the genitals, the anus, or the inside of the mouth or nose, or in particular to the cornea.

An effective amount of the therapeutic composition is determined based on the intended goal. The term “unit dose” or “dosage” refers to physically discrete units suitable for use in a subject, each unit containing a predetermined quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment and the potency, stability and toxicity of the particular therapeutic substance.

2. Routes of Administration

Formulations of the present disclosure are suitable for oral administration. However, the therapeutic compositions of the present disclosure may be administered via any common route so long as the target tissue is available via that route. This includes ocular, nasal, buccal, corneal, rectal, vaginal, or topical administration, and intradermal, subcutaneous, intramuscular, intraperitoneal or intravenous injection. As such, compositions would be formulated pharmaceutically in route-acceptable compositions that include physiologically acceptable carriers, buffers or other excipients.

As with dosing amounts, the timing of delivery (including intervals and total number of doses) depends on the judgment of the practitioner and are peculiar to each individual. Factors affecting dose include physical and clinical state of the patient, the route of administration, the intended goal of treatment and the potency, stability and toxicity of the particular therapeutic substance.

3. Combination Therapy

In many clinical situations, it is advisable to use a combination of distinct therapies. Thus, it is envisioned that, in addition to the therapies described above, one would also wish to provide to the patient another clinically approved pharmaceutical therapies. Examples of standard therapies are described above. Combinations may be achieved by administering a single composition or pharmacological formulation that includes both agents, or with two distinct compositions or formulations, at the same time, wherein one composition includes the agents of the present disclosure and the other includes the standard therapy. Alternatively, standard therapy may precede or follow the present agent treatment by intervals ranging from minutes to weeks to months. In embodiments where the treatments are applied separately, one would generally ensure that a significant period of time did not expire between the time of each delivery, such that the agents would still be able to exert an advantageously combined effect on the subject. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the agent of the present disclosure, or the standard therapy will be desired. Various combinations may be employed, where the present disclosure compound is “A” and the standard therapy is “B,” as exemplified below:
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B

Other combinations are contemplated as well.

c-erbA encodes the thyroid hormone receptor (TRO) and thus REV-ERBα is encoded by sequences of DNA on the opposite strand of the gene that encodes TRO. Both REV-ERBα and the closely related REV-ERBβ that was identified a few years after REV-ERBα, have an atypical LBD that lacks the carboxy-terminal activation function-2 (AF-2). Because the AF2 region recognizes coactivators that are required for transcriptional activation, these receptors have been generally characterized as unable to activate transcription. Indeed, the REV-ERBs are constitutive repressors of transcription, due their constant binding of corepressors such as the nuclear receptor co-repressor 1 (NCoR). The recruitment of corepressors to the target gene by the nuclear receptor (via the DNA response element) leads to repression of the target gene due to active histone deacetylation and condensation of the chromatin. Unlike many other nuclear receptors that function as obligate heterodimers (either homodimers or heterodimers with RXR) and recognize 2 copies of a core “half site” organized in either palindromic or repeated manner, REV-ERBs typically function as monomers and recognize a single 5′ extended AGGTCA “half site”. However, there have been reports of REV-ERB homodimers under some conditions. REV-ERBs have overlapping patterns of temporal and spatial expression, which is consistent with our current understanding that they display significant overlap in function. Both are widely expressed and interestingly both receptors exhibit a circadian pattern of expression that is essential for their role in circadian regulation of transcription.

Direct binding of heme to LBD of the REV-ERBs was demonstrated using several biochemical and biophysical methods, including mutation of a key residue that blocked heme binding and led to loss of transcriptional repressor function and derepression of target gene transcription. Moreover, reduction of intracellular heme levels decreased REV-ERB mediated repression of REV-ERB target genes, decreased interaction between REV-ERB and the NCoR-HDAC3 corepressor complex in cells, and impaired recruitment of NCoR to REV-ERB target gene promoters. These studies along with additional biophysical studies examining the affinity of heme for REV-ERB suggested that heme functions as an exchangeable ligand for the REV-ERBs. Crystal structures of REV-ERBs in the apo form and bound to heme have provided some insight into the molecular details of heme coordination by REV-ERBs and as to how REV-ERB might be targeted by synthetic ligands. The structure of heme-bound REV-ERBα LBD, revealed that the REV-ERB ligand-binding pocket is located in the same structural region as other nuclear receptors. The discovery that the REV-ERBs are ligand-regulated as well as considerable information regarding the therapeutic potential of targeting the REV-ERBs led to the discovery of synthetic REV-ERB ligands and their validation in several models of human disease including type 2 diabetes, obesity, heart disease, autoimmunity, chronic inflammation, anxiety, sleep disorders, cancer, muscular dystrophy and cognitive disorders.

REV-ERB is a key regulator of the oxidative capacity of skeletal muscle and mitochondrial biogenesis. REV-ERBα null mice had reduced mitochondrial content and oxidative function that resulted in reduced exercise capacity. REV-ERBs are also involved in adipogenesis. REV-ERBα expression is highly induced during adipogenesis and overexpression of REV-ERBα in 3T3-L1 cells results in increased expression of markers of adipogenesis including aP2, PPARγ and C/EBPα along with an increase in lipid accumulation. Furthermore, overexpression of REV-ERBα in these cells synergized with a PPARγ ligand to increase markers of adipogenesis. Although REV-ERBα expression is required for adipogenesis in cell-based models, Rev-erbα deficiency in vivo is associated with increased adiposity and increased weight gain due to a high fat diet. This apparent discrepancy may be due to a dual role for REV-ERBα in adipogenesis, where REV-ERBα expression is elevated in the initial stages of adipogenesis, but the protein is degraded in the late stages of the process to allow for efficient development of the fat cells. Interestingly, the degradation of REV-ERBα in late stage adipogenesis appears to be dependent upon increasing levels of the natural ligand for REV-ERBα. REV-ERBα deficient mice also display significant hepatic steatosis suggesting that pharmacological activation of REV-ERB may be useful in treating fatty liver and non-alcoholic steatohepatitis (NASH).

REV-ERBα has been demonstrated to regulate the production and release of the proinflammatory cytokine IL-6 in macrophages. Additionally, genome wide analysis of REV-ERBα and REV-ERBβ binding sites in macrophages revealed that these receptors were involved in a complex level of regulation of target genes suggesting an important role in this cell type. Beyond regulation of IL-6, REV-ERB has been demonstrated to play additional, essential roles in regulation of the innate immune system where it directly regulates expression of components of the NLRP3 inflammasome. REV-ERB suppresses the activity of the NLRP3 inflammasome by direct repression of the Nlrp3 and IIIb genes. REV-ERB agonists have been demonstrated to display efficacy in treatment of disease states where the NLRP3 inflammasome is abnormally elevated such as fulminant hepatitis and sepsis. A range of chronic inflammatory diseases have also been shown to be associated with elevated NLRP3 activity (Alzheimer's disease as well as other neurodegenerative disease, metabolic disease (obesity, NASH, type 2 diabetes), autoimmune diseases, gout, heart disease (atherosclerosis and heart failure), etc.) and these studies suggest that REV-ERB agonists may hold utility in treatment of these diseases as well. Given the opposing roles of the RORs and REV-ERBs, it is possible that that REV-ERBs may repress T_{H}17 cell development. Assessment of T_{H}17 cell differentiation is altered in REV-ERBα null mice indicated that synthetic REV-ERB ligands could be used to alter T_{H}17 development and thus treat autoimmunity.

Knock-down of REV-ERBα in hematopoietic cells followed by bone marrow transplantation into LDL receptor null mice revealed a critical role for REV-ERB in atherosclerosis. These mice displayed increased atherosclerotic plaque development while lipid levels were unaffected. The effect was attributed to altered macrophage function since overexpression of REV-ERBα was shown to lead to increased anti-inflammatory M2 macrophages. These data suggest that increasing REV-ERB repressive activity may be useful for the treatment/prevention of atherosclerosis, which was recently demonstrated using a REV-ERB agonist. Further studies have demonstrated that REV-ERB is very effective in inhibition of cholesterol synthesis and reducing LDL levels indicating that there may be additional advantages to development of a REV-ERB agonist for treatment of atherosclerosis. Recently, REV-ERB synthetic ligands have shown efficacy in treatment of heart failure and ischemic heart disease models.

The REV-ERBs are major regulatory components of the mammalian circadian clock. REV-ERBα is a key regulator of the cyclic expression of Bmal1. Two response elements are located in the Bmal1 promoter and Bmal1 expression is repressed by REV-ERBα. The circadian feedback loop exhibits additional complexity given that REV-ERBα expression is itself regulated by the BMAL1/CLOCK heterodimers via E box DNA response elements found within the REV-ERBα promoter. REV-ERBα^{-/-} mice exhibit aberrant expression of Bmal1 and exhibit alterations in the period and phase of their circadian locomotor behavior. REV-ERBβ^{-/-} mice display a much more subtle circadian phenotype, but the double REV-ERB null mice are arrhythmic and display a similar phenotype to the Bmal1^{-/-}, Cry1^{-/-}/Cry2^{-/-}, and Per1^{-/-}/Per2^{-/-} mice. In fact, the expression of the REV-ERB genes is driven by E-boxes in their promoter elements that are similar to that that drive the circadian expression of the Cry and Per genes. These data suggest that the REV-ERBs should be considered core clock genes rather than components of an accessory loop that only modulates the pattern of expression of the core clock genes. Given the role of the REV-ERBs in regulation of the clock, several have demonstrated the utility of pharmacologically targeting the REV-ERBs as a method to module clock associated diseases including sleep disorders and metabolic disorders. Additionally, with the well characterized like between aberrant circadian rhythms and cancer, there has been a number of investigators who have examined the efficacy of REV-ERB ligands in animal models of cancer. Indeed, REV-ERB agonists have been shown to have anti-cancer activity in models of glioblastoma and breast cancer.

Without further elaboration, it is believed that one skilled in the art using the preceding description can utilize the present disclosure to its fullest extent. The following non-limiting Examples are illustrative of the disclosure.

The following examples are included to further illustrate various aspects of the disclosure. It should be appreciated by those of skill in the art that the techniques disclosed in the examples which follow represent techniques and/or compositions discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra (DMSO-d6, 400 MHz unless indicated otherwise) are reported in ppm downfield from tetramethylsilane; “s” means singlet, “d” means doublet, “t” means triplet, “dd” means doublet of doublets, “dt” means doublet of triplets, “q” means quartet, “m” means multiplet, and “br s” means broad singlet.

### Synthesis Examples

### Example 1: Preparation of 2-([1,1′-biphenyl]-2-yloxy)-9-methyl-9H-purine (CDD-1285

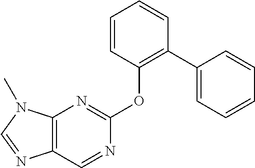 [SMILES: Cn1cnc2cnc(Oc3ccccc3-c3ccccc3)nc21]

### Step 1: Preparation of 2-chloro-9-methyl-9H-purine

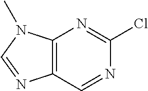

A mixture of 2-chloro-9H-purine (200 mg, 1.30 mmol), K₂CO₃ (540 mg, 3.9 mmol), and iodomethane (370 mg, 2.6 mmol) was stirred at 0° C. for 2 h. Water (50 mL) was added and the mixture extracted with DCM (3×100 mL). The combined organic layers were washed with brine and concentrated in vacuo. The residue was purified by Prep-HPLC to afford the desired crude material (80 mg, 37%) as a white solid: MS (ES): m/z=169 (M+H).

### Step 2: Preparation of 2-([1,1′-biphenyl]-2-yloxy)-9-methyl-9H-purine

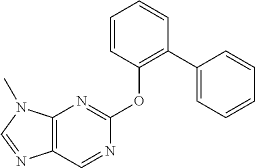 [SMILES: Cn1cnc2cnc(Oc3ccccc3-c3ccccc3)nc21]

A mixture of [1,1′-biphenyl]-2-ol (67 mg, 0.39 mmol), 2-chloro-9-methyl-9H-purine (60 mg, 0.36 mmol) and K₂CO₃ (148 mg, 1.10 mmol) in DMF (2 mL) was heated at 110° C. overnight. After cooling, water (50 mL) was added and the mixture extracted with DCM (3×100 mL). The combined DCM layers were washed with brine and concentrated in vacuo. The residue was purified by Prep-HPLC to afford the desired product (70 mg, 65%) as an off-white solid: ¹H NMR (400 MHz, DMSO-d6) δ 8.81 (s, 1H), 8.37 (s, 1H), 7.52-7.45 (m, 1H), 7.45-7.39 (m, 3H), 7.36 (td, J=7.5, 1.3 Hz, 1H), 7.33-7.27 (m, 2H), 7.24 (ddd, J=7.4, 4.2, 1.2 Hz, 2H), 3.68 (s, 3H); MS (ES): m/z=302 (M+H).

### Example 2: Preparation of 9-methyl-2-(2-(pyridin-3-yl)-5-(trifluoromethyl)phenoxy)-9H-purine

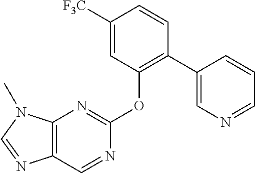 [SMILES: Cn1cnc2cnc(Oc3cc(C(F)(F)F)ccc3-c3cccnc3)nc21]

### Step 1: Preparation of 2-([1,1′-biphenyl]-2-yloxy)-9-methyl-9H-purine

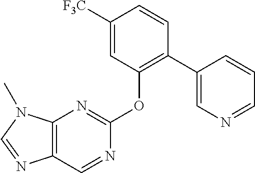 [SMILES: Cn1cnc2cnc(Oc3cc(C(F)(F)F)ccc3-c3cccnc3)nc21]

A mixture of 2-(pyridin-3-yl)-5-(trifluoromethyl)phenol (125 mg, 0.523 mmol), 2-chloro-9-methyl-9H-purine (80 mg, 0.48 mmol), and K₂CO₃ (197 mg, 1.43 mmol) in DMF (2 mL) was heated at 110° C. overnight. After cooling water was added (50 mL) and the mixture extracted with DCM (3×100 mL). The combined DCM layers were washed with brine and concentrated in vacuo. The residue was purified by Prep-HPLC to afford the desired material (65 mg, 37%) as a gray solid: ¹H NMR (400 MHz, DMSO-d6) δ: 8.84 (s, 1H), 8.67 (dd, J=2.3, 0.7 Hz, 1H), 8.50 (dd, J=4.8, 1.6 Hz, 1H), 8.42 (s, 1H), 7.95-7.87 (m, 1H), 7.86-7.74 (m, 3H), 7.40 (ddd, J=7.9, 4.8, 0.8 Hz, 1H), 3.69 (s, 3H); MS (ES): m/z=372 (M+H).

### Example 3: Preparation of 5-([1,1′-biphenyl]-2-yloxy)-3-methyl-3H-imidazo[4,5-b]pyridine (CDD-1316

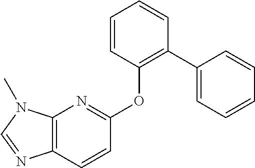 [SMILES: Cn1cnc2ccc(Oc3ccccc3-c3ccccc3)nc21]

### Step 1: Preparation of 6-chloro-N-methyl-3-nitropyridin-2-amine

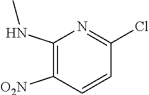

A solution of 2,6-dichloro-3-nitropyridine (5.00 g, 30.6 mmol) in ethanol (152 mL) was added to a solution of methylamine in ethanol (40%, 5.29 g, 46.0 mmol) and the mixture was stirred at room temperature overnight. The solvent was removed in vacuo and the crude was purified by flash chromatography (ethyl acetate/petroleum ether, 0-60%) to provide the expected product as a yellow solid (2.52 g, 44%); MS (ES) m/z=188 (M+H).

### Step 2: Preparation of 6-chloro-N²-methylpyridine-2,3-diamine

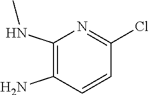

6-Chloro-N-methyl-3-nitropyridin-2-amine (2.58 g, 13.8 mmol) was dissolved in hydrochloric acid (35%, 70 mL). Stannous chloride (13.0 g, 68.8 mmol) was added and then the mixture was heated at reflux overnight. The mixture was cooled to room temperature and adjusted to pH=7 to 8. The resultant solid was filtered via Celite. The filtrate was extracted with ethyl acetate (3×100 mL). The combined the organic phase was washed with water (100 mL), brine (100 mL), dried over Na₂SO₄, filtered, and concentrated to give the expected crude product (2.3 g, 100%) as a brown solid: MS (ES) m/z=158 (M+H).

### Step 3: Preparation of 5-chloro-3-methyl-3H-imidazo[4,5-b]pyridine

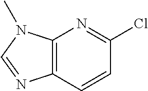

To a mixture of 6-chloro-N²-methylpyridine-2,3-diamine (2.30 g, 14.6 mmol) and trimethoxymethane (73 mL) was added TsOH (8.5 mg, 1.5 mmol) and the mixture heated at reflux overnight. After cooling the mixture was concentrated in vacuo, and the crude was purified by flash chromatography (ethyl acetate/petroleum ether: 0-90%) to give the expected product (1.1 g, 45%) as a yellow solid: MS (ES) m/z=168 (M+H).

### Step 4: Preparation of 5-([1,1′-biphenyl]-2-yloxy)-3-methyl-3H-imidazo[4,5-b]pyridine

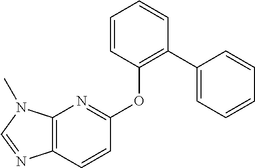 [SMILES: Cn1cnc2ccc(Oc3ccccc3-c3ccccc3)nc21]

To a mixture of 5-chloro-3-methyl-3H-imidazo[4,5-b]pyridine (300 mg, 1.79 mmol) in 1-methyl-2-pyrrolidinone (9 mL) was added K₃PO₄ (760 mg, 0.900 mmol), [1,1′-biphenyl]-2-ol (60 mg, 3.58 mmol), cyclohexane-1,2-diamine (102 mg, 0.180 mmol), and CuI (34 mg, 0.18 mmol). The mixture was flushed with nitrogen for 10 min and then heated at 230° C. for 8 h. After cooling the mixture was filtered and concentrated. The residue was purified by reverse phase column (MeCN/water: 0-95%) to give the expected product (105 mg, 20%) as a white solid: ¹H NMR (400 MHz, DMSO-d6) δ: 8.23 (s, 1H), 8.02 (d, J=8.5 Hz, 1H), 7.54-7.46 (m, 3H), 7.45-7.38 (m, 1H), 7.34 (dd, J=8.1, 6.8 Hz, 3H), 7.30-7.22 (m, 1H), 7.18 (dd, J=8.1, 1.1 Hz, 1H), 6.75 (d, J=8.5 Hz, 1H), 3.63 (s, 3H); MS (ES) m/z=302 (M+H)

### Example 4: Preparation of 3-methyl-5-(3′-methylbiphenyl-2-yloxy)-3H-imidazo[4,5-b]pyridine (CDD-1459

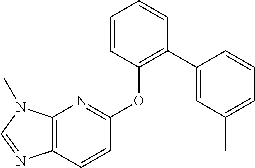 [SMILES: Cc1cccc(-c2ccccc2Oc2ccc3ncn(C)c3n2)c1]

### Step 1: Preparation of 3′-methylbiphenyl-2-ol

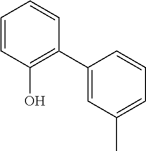

A mixture of 3-bromo-3-toluene (1.00 g, 5.88 mmol), K₂CO₃ (1.62 g, 11.8 mmol), 2-hydroxyphenylboronic acid (1.14 g, 8.24 mmol), and PdCl₂(PPh₃)₄ (430 mg, 0.59 mmol) in H₂O (1.5 mL) and dioxane (15 mL) was flushed with N₂ and then heated at 90° C. overnight. The mixture was diluted with water and extracted EtOAc. The combined EtOAc layers were concentrated in vacuo. The residue was purified chromatography on silica gel to afford the expected product (0.80 g, 74%) as a yellow oil: MS (ES): m/z=185 (M+H).

### Step 2: Preparation of N-methyl-6-(3′-methylbiphenyl-2-yloxy)-3-nitropyridin-2-amine

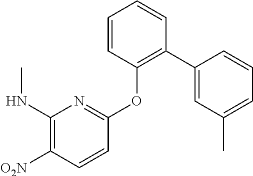 [SMILES: CNc1nc(Oc2ccccc2-c2cccc(C)c2)ccc1[N+](=O)[O-]]

A mixture of 3′-methylbiphenyl-3-ol (300 mg, 1.63 mmol), 6-chloro-N-methyl-3-nitropyridin-2-amine (277 mg, 1.48 mmol), and K₂CO₃ (614 mg, 4.45 mmol) in DMF (2 mL) was heated at 70° C. for 6 h. The mixture was cooled, water (15 mL) was added, and the mixture extracted with EtOAc (2×15 mL). The combined EtOAc layers were dried and concentrated in vacuo. The residue was purified by chromatography to afford the expected product (256 mg, 47%) as a yellow oil: MS (ES): m/z=336 (M+H).

### Step 3: Preparation of N²-methyl-6-(3′-methylbiphenyl-2-yloxy)pyridine-2,3-diamine

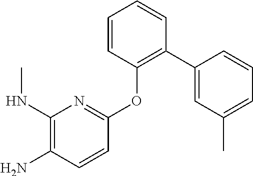 [SMILES: CNc1nc(Oc2ccccc2-c2cccc(C)c2)ccc1N]

To a solution of N-methyl-6-(3′-methylbiphenyl-2-yloxy)-3-nitropyridin-2-amine (256 mg, 0.76 mmol) in MeOH (20 mL) was added Pd/C (100 mg, 0.076 mmol) under a N₂ atmosphere and the mixture was stirred under hydrogen at room temperature overnight. The mixture was filtered and the filtrate was concentrated in vacuo to afford the expected product (110 mg, 47%) as a yellow oil: MS (ES): m/z=306 (M+H).

### Step 4: Preparation of 3-methyl-5-(3′-methylbiphenyl-2-yloxy)-3H-imidazo[4,5-b]pyridine

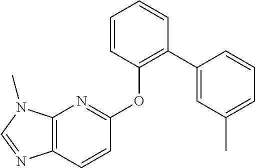 [SMILES: Cc1cccc(-c2ccccc2Oc2ccc3ncn(C)c3n2)c1]

A mixture of N²-methyl-6-(3′-methylbiphenyl-2-yloxy)pyridine-2,3-diamine (110 mg, 0.360 mmol) in trimethyl orthoformate (15 mL) was added toluene sulfonic acid (20 mg, 0.036 mmol) and the mixture heated at 102° C. overnight. The reaction mixture was cooled to room temperature and concentrated in vacuo. The residue was purified by Prep-HPLC to afford the expected product (40 mg, 35%) as a yellow oil: ¹H NMR (400 MHz, DMSO-d6) δ: 7.9-7.92 (d, J=9.6 Hz, 1H), 7.85 (d, J=9.6 Hz, 1H), 7.02-7.48 (m, 9H), 6.63-6.65 (d, J=9.6 Hz, 1H), 3.73 (s, 3H), 2.29 (s, 3H), MS (ES): m/z=316 (M+H).

### Example 5: Preparation of 5-(3′-fluorobiphenyl-2-yloxy)-3-methyl-3H-imidazo[4,5-b]pyridine (CDD-1496

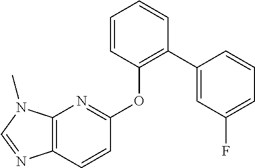 [SMILES: Cn1cnc2ccc(Oc3ccccc3-c3cccc(F)c3)nc21]

### Step 1: Preparation of 6-(2-bromophenoxy)-N-methyl-3-nitropyridin-2-amine

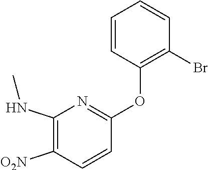 [SMILES: CNc1nc(Oc2ccccc2Br)ccc1[N+](=O)[O-]]

To a solution of 6-(2-bromophenoxy)-N-methyl-3-nitropyridin-2-amine (400 mg, 1.20 mmol) in water (1 mL) and dioxane (8 mL) was added 3-fluorophenylboronic acid (242 mg, 1.70 mmol), K₂CO₃ (340 mg, 2.40 mmol), and PdCl₂(dppf) (60 mg, 0.06 mmol). The mixture was flushed with N₂ and then heated at 90° C. for 8 h. After cooling, water (20 mL) was added and the mixture extracted with EtOAc (2×20 mL). The combined EtOAc layers were concentrated under reduced pressure. The residue was purified by silica gel column to afford 6-(3′-fluorobiphenyl-2-yloxy)-N-methyl-3-nitropyridin-2-amine (115 mg, 27%) as a yellow oil: MS (ES) m/z=349.9 (M+H).

### Step 2: Preparation of 6-(3′-fluorobiphenyl-2-yloxy)-N²-methylpyridine-2,3-diamine

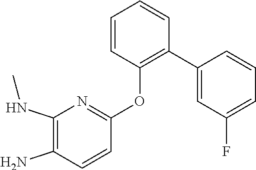 [SMILES: CNc1nc(Oc2ccccc2-c2cccc(F)c2)ccc1N]

To a solution of 6-(2-bromophenoxy)-N-methyl-3-nitropyridin-2-anine (115 ng, 0.34 mmol) in MeOH (15 mL) was added Pd/C (95 mg) under a N₂ atmosphere, and the mixture was stirred under a H₂ atmosphere at room temperature for 4 h. The mixture was filtered and the filtrate was concentrated in vacuo to afford 6-(3′-fluorobiphenyl-2-yloxy)-N²-methylpyridine-2,3-diamine (90 mg, 86%) as a brown oil: MS (ES) m/z=310.1 (M+H).

### Step 3: Preparation of 5-(3′-fluorobiphenyl-2-yloxy)-3-methyl-3H-imidazo[4,5-b]pyridine

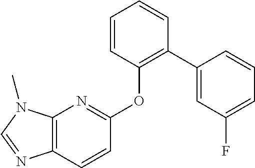 [SMILES: Cn1cnc2ccc(Oc3ccccc3-c3cccc(F)c3)nc21]

To a solution of 6-(3′-fluorobiphenyl-2-yloxy)-N²-methylpyridine-2,3-diamine (90 mg, 0.29 mmol) in trimethoxymethane (3 mL) was added p-TsOH (5 mg, 0.03 mmol) and the mixture heated at 102° C. overnight under a N₂ atmosphere. After cooling, the mixture was concentrated to dryness and the residue was purified by prep-HPLC to afford 5-(3′-fluorobiphenyl-2-yloxy)-3-methyl-3H-imidazo[4,5-b]pyridine (12 mg, 13%) as a yellow oil: ¹H NMR (400 MHz, DMSO-d6) δ 8.48 (s, 1H), 8.09 (d, J=8.6 Hz, 1H), 7.55 (dd, J=7.6, 1.7 Hz, 1H), 7.50-7.44 (m, 1H), 7.41-7.27 (m, 4H), 7.22 (dd, J=8.1, 1.1 Hz, 1H), 7.11 (t, J=7.9 Hz, 1H), 6.86 (d, J=8.6 Hz, 1H), 3.65 (s, 3H); MS (ES) m/z=320 (M+H).

### Example 6: Preparation of 3-methyl-5-(2-(pyridin-3-yl)phenoxy)-3H-imidazo[4,5-b]pyridine (CDD-1317

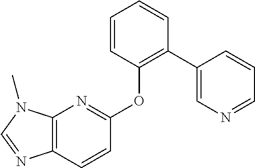 [SMILES: Cn1cnc2ccc(Oc3ccccc3-c3cccnc3)nc21]

### Step 1: Preparation of 6-chloro-N-methyl-3-nitropyridin-2-amine

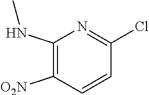

To a solution of 2,6-dichloropyridin-3-amine (5.00 g, 30.6 mmol) in ethanol (152 mL) was added a solution of methylamine in ethanol (40%, 5.29 g, 46.0 mmol) and the mixture stirred at room temperature overnight. The solvent was removed in vacuo and the crude was purified by flash chromatography (ethyl acetate/petroleum ether 0-60%) to give the expected product as a yellow solid (2.52 g, 44%): MS (ES) m/z=188 (M+H).

### Step 2: Preparation of N-methyl-3-nitro-6-(2-(pyridin-3-yl)phenoxy)pyridin-2-amine

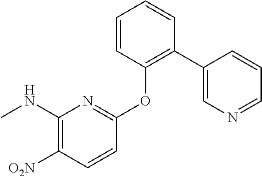 [SMILES: CNc1nc(Oc2ccccc2-c2cccnc2)ccc1[N+](=O)[O-]]

To a solution of 6-chloro-N-methyl-3-nitropyridin-2-amine (1.00 g, 5.30 mmol) in N,N-dimethylformamide (25 mL) was added 2-(pyridin-3-yl)phenol (913 mg, 5.30 mmol) and potassium carbonate (1.47 g, 10.7 mmol). The mixture was heated at 80° C. for 3 h. After cooling the mixture was partitioned between water (50 mL) and ethyl acetate (3×40 mL). The combined organic phase was washed with water (30 mL), brine (30 mL), dried over Na₂SO₄, filtered, and concentrated in vacuo. The crude was purified by flashed chromatography (ethyl acetate/petroleum ether: 0-95%) to give the expected product (890 mg, 49%) as an orange solid: MS (ES) m/z=323 (M+H).

### Step 3: Preparation of N²-methyl-6-(2-(pyridin-3-yl)phenoxy)pyridine-2,3-diamine

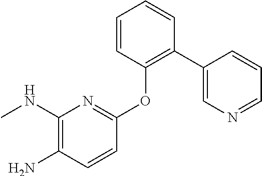 [SMILES: CNc1nc(Oc2ccccc2-c2cccnc2)ccc1N]

To a suspension of N-methyl-3-nitro-6-(2-(pyridin-3-yl)phenoxy)pyridin-2-amine (840 mg, 2.60 mmol) in CH₃OH (13 mL) was added Pd/C (10%, 200 mg) and the mixture stirred under a hydrogen atmosphere overnight. The mixture was filtered through Celite. The filtrate was concentrated in vacuo to give the expected product (486 mg, 56%) as a black solid: MS (ES) m/z=293 (M+H).

### Step 4: Preparation of 3-methyl-5-(2-(pyridin-3-yl)phenoxy)-3H-imidazo[4,5-b]pyridine

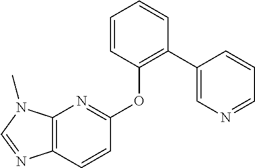 [SMILES: Cn1cnc2ccc(Oc3ccccc3-c3cccnc3)nc21]

To a mixture of N²-methyl-6-(2-(pyridin-3-yl)phenoxy)pyridine-2,3-diamine (486 mg, 1.65 mmol) in trimethoxymethane (9 mL) was added 4-methylbenzenesulfonic acid (8.5 mg, 0.05 mmol). The mixture was then heated at reflux for 3 h. After cooling the mixture was concentrated in vacuo, and the crude purified by reverse phase column (aqueous NaHCO₃/acetonitrile: 0-95%) to give the expected product (200 mg, 45%) as a yellow solid: ¹H NMR (400 MHz, DMSO-d6) δ: 8.68 (dd, J=2.3, 0.8 Hz, 1H), 8.45 (dd, J=4.8, 1.6 Hz, 1H), 8.24 (s, 1H), 8.04 (d, J=8.5 Hz, 1H), 7.94-7.86 (m, 1H), 7.57 (dd, J=7.6, 1.7 Hz, 1H), 7.52-7.43 (m, 1H), 7.42-7.32 (m, 2H), 7.23 (dd, J=8.1, 1.1 Hz, 1H), 6.79 (d, J=8.5 Hz, 1H), 3.62 (s, 3H); MS (ES) m/z=303 (M+H).

### Example 7: Preparation of 6-([1,1′-biphenyl]-2-yloxy)-1-methyl-1H-pyrazolo[3,4-d]pyrimidine (CDD-1304

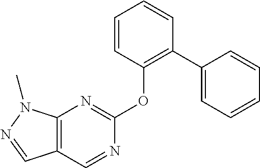 [SMILES: Cn1ncc2cnc(Oc3ccccc3-c3ccccc3)nc21]

### Step 1: Preparation of 6-chloro-1-methyl-1H-pyrazolo[3,4-d]pyrimidine

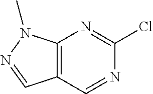

To a mixture of 6-chloro-1H-pyrazolo[3,4-d]pyrimidine (100 mg, 0.650 mmol) and K₂CO₃ (270 mg, 1.95 mmol) in DMF (2 mL) was added iodomethane (184 mg, 1.30 mmol) and the mixture stirred at 0° C. for 3 h. Water (30 mL) was added and the mixture extracted with DCM (3×50 mL). The combined DCM layers were washed with brine and concentrated in vacuo. The residue was purified by column chromatography to afford the desired material (82 mg, 75%) as a white solid: MS (ES): m/z=169 (M+H).

### Step 2: Preparation of 6-([1,1′-biphenyl]-2-yloxy)-1-methyl-1H-pyrazolo[3,4-d]pyrimidine

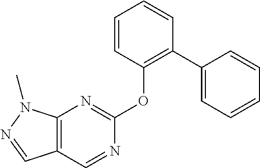 [SMILES: Cn1ncc2cnc(Oc3ccccc3-c3ccccc3)nc21]

A mixture of [1,1′-biphenyl]-2-ol (91 mg, 0.54 mmol), 6-chloro-1-methyl-1H-pyrazolo[3,4-d]pyrimidine (91 mg, 0.49 mmol), and K₂CO₃ (202 mg, 1.46 mmol) in DMF (2 mL) was heated at 110° C. overnight. After cooling, water (50 mL) was added and the mixture extracted with DCM (3×100 mL). The combined DCM layers were washed with brine and concentrated in vacuo. The residue was purified by Prep-HPLC to afford the desired material (95 mg, 65%) as a white solid: ¹H NMR (400 MHz, DMSO-d6) δ: 9.03 (s, 1H), 8.22 (s, 1H), 7.44 (dqd, J=21.4, 7.4, 1.4 Hz, 1H), 7.31 (dd, J=9.6, 4.4 Hz, 1H), 7.23 (t, J=7.3 Hz, 1H), 3.83 (s, 1H); MS (ES): m/z=303 (M+H).

### Example 8: Preparation of 1-methyl-6-(3′-methylbiphenyl-2-yloxy)-1H-pyrazolo[3,4-b]pyridine (CDD-1450

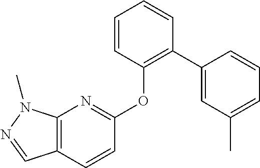 [SMILES: Cc1cccc(-c2ccccc2Oc2ccc3cnn(C)c3n2)c1]

### Step 1: Preparation of 6-chloro-1-methyl-1H-pyrazolo[3,4-b]pyridine

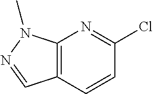

To a solution of 6-chloro-1-methyl-1H-pyrazolo[3,4-b]pyridine (200 mg, 1.30 mmol) in DMF (5 mL) was added sodium hydride (60% mineral oil dispersion, 78 mg, 1.95 mmol) at 0° C. under N₂. The mixture was stirred at 0° C. for 30 min and then iodomethane (0.10 mL, 1.6 mmol) was added. The mixture was stirred at room temperature for 2 h, then poured to cold water (5 mL), and the mixture extracted with ethyl acetate (2×10 mL). The organic phase was dried over Na₂SO₄ and concentrated under reduced pressure. The residue was purified by flash chromatography (ethyl acetate/petroleum ether: 0-60%) to give the expected product (160 mg, 73%) as a yellow solid: MS (ES): m/z=168.1 (M+H).

### Step 2: Preparation of 1-methyl-6-(3′-methylbiphenyl-2-yloxy)-1H-pyrazolo[3,4-b]pyridine

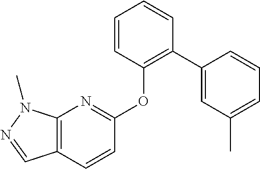 [SMILES: Cc1cccc(-c2ccccc2Oc2ccc3cnn(C)c3n2)c1]

To a solution of 6-chloro-1-methyl-1H-pyrazolo[3,4-b]pyridine (80 mg, 0.48 mmol) in DMF (3 mL) was added 3′-methylbiphenyl-2-ol (97 mg, 0.52 mmol) and K₂CO₃ (199 mg, 1.43 mmol). The mixture was heated at 130° C. overnight. After cooling the mixture was filtered and concentrated. The residue was purified by prep-HPLC to give the expected product (21.9 mg, 15%) as a yellow oil: ¹H NMR (400 MHz, DMSO-d6) δ: 8.13 (d, J=8.6 Hz, 1H), 7.98 (s, 1H), 7.53-7.42 (m, 2H), 7.37 (td, J=7.4, 1.3 Hz, 1H), 7.29 (dd, J=8.0, 1.2 Hz, 1H), 7.26-7.15 (m, 3H), 7.04 (d, J=7.3 Hz, 1H), 6.70 (d, J=8.6 Hz, 1H), 3.76 (s, 3H); MS (ES): m/z=316.1 (M+H).

### Example 9: Preparation of N-methyl-2′-(1-methyl-1H-pyrazolo[3,4-d]pyrimidin-6-yloxy)biphenyl-3-amine (CDD-1430

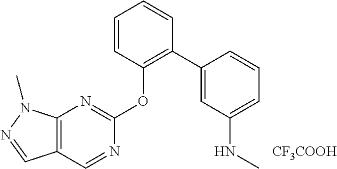 [SMILES: CNc1cccc(-c2ccccc2Oc2ncc3cnn(C)c3n2)c1 ; "CF3COOH"]

### Step 1: Preparation of 6-chloro-N-methyl-3-nitropyridin-2-amine

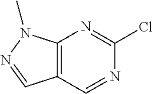

To a solution of 6-chloro-1H-pyrazolo[3,4-d]pyrimidine (300 mg, 1.94 mmol) in DMF (5 mL) at 0° C. under N₂ was added sodium hydride (60% mineral oil dispersion, 116 mg, 4.85 mmol). The mixture was stirred at 0° C. for 30 min. Iodomethane (0.15 mL, 2.3 mmol) was added dropwise to the mixture which was stirred at room temperature for 1.5 h. Cold water (10 mL) was added and the mixture extracted with ethyl acetate (2×10 mL). The organic phase was dried over Na₂SO₄ and concentrated in vacuo. The residue was purified by flash chromatography (ethyl acetate/petroleum ether: 0-60%) to give the expected product (170 mg, 52%) as a yellow solid: MS (ES): m/z=169.0 (M+H).

### Step 2: Preparation of tert-butyl methyl (2′-(1-methyl-1H-pyrazolo[3,4-d]pyrimidin-6-yloxy)biphenyl-3-yl)carbamate

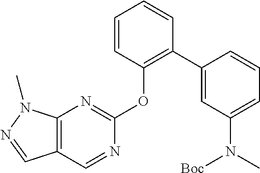 [SMILES: CN(C(=O)OC(C)(C)C)c1cccc(-c2ccccc2Oc2ncc3cnn(C)c3n2)c1]

To a solution of 6-chloro-N-methyl-3-nitropyridin-2-amine (90 mg, 0.53 mmol) in DMF (3 mL) was added tert-butyl 2′-hydroxybiphenyl-3-yl(methyl)carbamate (176 mg, 0.587 mmol), and K₂CO₃ (221 mg, 1.60 mmol). The mixture was heated at 130° C. overnight. After cooling the mixture was poured onto water (5 mL) and extracted with ethyl acetate (2×10 mL). The organic phase was concentrated under reduced pressure. The residue was purified by chromatography (ethyl acetate in petroleum ether: 0-80%) to give the product (120 mg, 52%) as a yellow oil: MS (ES): m/z=431.8 (M+H).

### Step 3: Preparation of N-methyl-2′-(1-methyl-1H-pyrazolo[3,4-d]pyrimidin-6-yloxy)biphenyl-3-amine

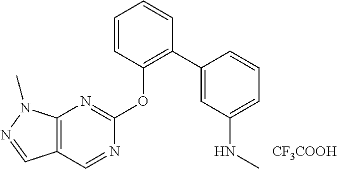 [SMILES: CNc1cccc(-c2ccccc2Oc2ncc3cnn(C)c3n2)c1 ; "CF3COOH"]

Trifluoroacetic acid (0.5 mL) was added to a solution of tert-butyl methyl (2′-(1-methyl-1H-pyrazolo[3,4-d]pyrimidin-6-yloxy)biphenyl-3-yl)carbamate (120 mg, 0.280 mmol) in DCM (2 mL). The mixture was stirred at room temperature for 1.5 h and then concentrated under reduced pressure. The residue was purified by prep-HPLC to get the expected product (69 mg, 56%) as a yellow solid: ¹H NMR (400 MHz, DMSO-d6) δ: 9.05 (s, 1H), 8.24 (s, 1H), 7.45 (ddd, J=15.4, 7.5, 1.8 Hz, 2H), 7.37 (td, J=7.4, 1.2 Hz, 1H), 7.28 (dd, J=7.9, 1.1 Hz, 1H), 6.70 (d, J=50.4 Hz, 3H), 2.55 (d, J=21.0 Hz, 3H); MS (ES): m/z=331.8 (M+H).

### Example 10: Preparation of 1-methyl-6-(2-(pyridin-3-yl)phenoxy)-1H-pyrazolo[3,4-d]pyrimidine (CDD-1427

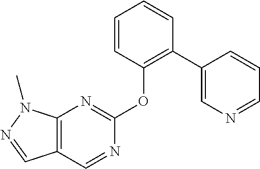 [SMILES: Cn1ncc2cnc(Oc3ccccc3-c3cccnc3)nc21]

### Step 1: Preparation of 6-chloro-N-methyl-3-nitropyridin-2-amine

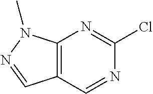

To a solution of 6-chloro-1H-pyrazolo[3,4-d]pyrimidine (80 mg, 0.52 mmol) in DMF (2 mL) at 0° C. under N₂ was added sodium hydride (60% mineral oil dispersion, 19 mg, 0.78 mmol). The mixture was stirred at 0° C. for 30 min. Iodomethane (0.04 mL, 0.62 mmol) was added to the mixture. After the mixture stirred at room temperature for 1.5 h, cold water (5 mL) was added, and the mixture extracted with ethyl acetate (2×10 mL). The organic phase was dried over Na₂SO₄, filtered, and concentrated under reduced pressure. The residue was purified by flash chromatography (ethyl acetate/petroleum ether: 0-60%) to give the expected product as a yellow solid (40 mg, 46%): MS (ES) m/z=169.0 (M+H).

### Step 2: Preparation of 1-methyl-6-(2-(pyridin-3-yl)phenoxy)-1H-pyrazolo[3,4-d]pyrimidine

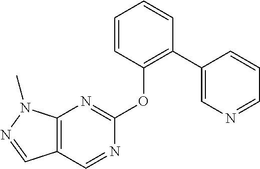 [SMILES: Cn1ncc2cnc(Oc3ccccc3-c3cccnc3)nc21]

To a mixture of 6-chloro-N-methyl-3-nitropyridin-2-amine (40 mg, 0.24 mmol) in DMF (3 mL) was added 2-(pyridin-3-yl)phenol (45 mg, 0.26 mmol) and K₂CO₃ (98 mg, 0.71 mmol). The mixture was heated at 130° C. overnight. After cooling the mixture was filtered and purified by prep-HPLC to afford the expected product as a gray solid (31.7 mg, 44%): ¹H NMR (400 MHz, DMSO-d6) δ: 9.04 (s, 1H), 8.62 (d, J=1.7 Hz, 1H), 8.44 (dd, J=4.8, 1.6 Hz, 1H), 8.24 (s, 1H), 7.90-7.78 (m, 1H), 7.60-7.48 (m, 2H), 7.44 (td, J=7.5, 1.2 Hz, 1H), 7.39-7.29 (m, 2H), 3.84 (s, 3H); MS (ES) m/z=304 (M+H).

### Example 11: Preparation of 1-methyl-6-(2-(pyridin-3-yl)-5-(trifluoromethyl)phenoxy)-1H-pyrazolo[3,4-d]pyrimidine (CDD-1429

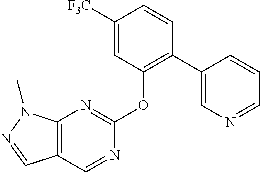 [SMILES: Cn1ncc2cnc(Oc3cc(C(F)(F)F)ccc3-c3cccnc3)nc21]

To a solution of 6-chloro-1-methyl-1H-pyrazolo[3,4-d]pyrimidine (40 mg, 237 umoL) in N,N-dimethylformamide (2 mL) was added 2-(pyridin-3-yl)-5-(trifluoromethyl)phenol (68.1 mg, 285 umol) and potassium carbonate (98.4 mg, 712 umol). The mixture was heated at 130° C. overnight. After cooling the crude was purified by Prep-HPLC (A: water (0.06% TFA); B: MeCN; 20-50% B) to give the expected product (65 mg, 73%) as a white solid: ¹H NMR (400 MHz, DMSO-d6) δ: 9.07 (s, 1H), 8.88 (s, 1H), 8.67 (d, J=4.6 Hz, 1H), 8.30-8.21 (m, 2H), 7.95-7.87 (m, 2H), 7.85 (d, J=8.2 Hz, 1H), 7.69 (dd, J=7.9, 5.2 Hz, 1H), 3.86 (s, 3H); MS (ES): m/z 372 (M+H).

### Example 12: Preparation of 6-([1,1′-biphenyl]-2-yloxy)-1-methyl-1H-pyrazolo[3,4-b]pyridine (CDD-1309

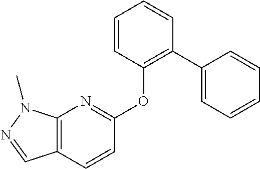 [SMILES: Cn1ncc2ccc(Oc3ccccc3-c3ccccc3)nc21]

### Step 1: Preparation of ethyl 3-ethoxy-3-((1-methyl-1H-pyrazol-5-yl)amino)propanoate

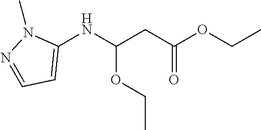 [SMILES: CCOC(=O)CC(Nc1ccnn1C)OCC]

Sodium metal (380 mg, 16.5 mmol) was dissolved in EtOH (12 mL) at 40° C. under a N₂ atmosphere. Once dissolved 1-methyl-1H-pyrazol-5-amine (400 mg, 4.12 mmol) was added in one portion. The reaction mixture was stirred for 1 h and then ethyl propiolate (808 mg, 8.24 mmol) was added dropwise over 20 min. The mixture was heated at reflux for 16 h, cooled to room temperature, and then concentrated in vacuo. Water (50 mL) was added and the mixture extracted with EtOAc (3×50 mL). The combined EtOAc layers were washed with brine and concentrated in vacuo. The residue was purified by silica gel column to afford the desired material (456 mg, 46%) as a brown solid: MS (ES): m/z=242 (M+H).

### Step 2: Preparation of 1-methyl-1H-pyrazolo[3,4-b]pyridin-6(7H)-one

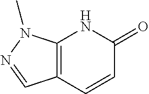

A solution of ethyl 3-ethoxy-3-((1-methyl-1H-pyrazol-5-yl)amino)propanoate (400 mg, 1.66 mmol) in acetic acid (5 mL) was heated at reflux for 3 h, and after cooling was then concentrated in vacuo. Water (30 mL) was added and the mixture extracted with DCM (3×50 mL). The combined DCM layers were washed with brine and concentrated in vacuo. The residue was purified by silica gel column to afford the desired material (215 mg, 87%) as a yellow solid: MS (ES): m/z=150 (M+H).

### Step 3: Preparation of 6-chloro-1-methyl-1H-pyrazolo[3,4-b]pyridine

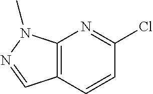

A mixture of 1-methyl-1H-pyrazolo[3,4-b]pyridin-6(7H)-one (150 mg, 1.00 mmol) and POCl₃ (3 mL) was heated at 90° C. overnight. The mixture was cooled to room temperature, poured onto ice, and extracted with EtOAc (3×50 mL). The combined EtOAc layers were washed with brine, and concentrated in vacuo. The residue was purified by column chromatography to afford the desired material (93 mg, 55%) as a yellow solid: MS (ES): m/z=168 (M+H).

### Step 4: Preparation of 6-([1,1′-biphenyl]-2-yloxy)-1-methyl-1H-pyrazolo[3,4-b]pyridine

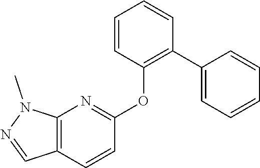 [SMILES: Cn1ncc2ccc(Oc3ccccc3-c3ccccc3)nc21]

A mixture of [1,1′-biphenyl]-2-ol (112 mg, 0.66 mmol), 6-chloro-1-methyl-1H-pyrazolo[3,4-b]pyridine (100 mg, 0.60 mmol), and K₂CO₃ (250 mg, 1.8 mmol) in DMF (1.5 mL) was heated at 130° C. overnight. After cooling water (50 mL) was added and the mixture extracted with DCM (3×100 mL). The combined DCM layers were washed with brine and concentrated in vacuo. The residue was purified by Prep-HPLC to afford the desired material (70 mg, 39%) as an off-white solid: ¹H NMR (400 MHz, DMSO-d6) δ: 8.13 (d, J=8.6 Hz, 1H), 7.98 (s, 1H), 7.52 (dd, J=7.5, 1.7 Hz, 1H), 7.50-7.41 (m, 3H), 7.39 (td, J=7.5, 1.3 Hz, 1H), 7.31 (ddd, J=8.0, 5.2, 1.5 Hz, 3H), 7.27-7.21 (m, 1H), 6.72 (d, J=8.6 Hz, 1H), 3.76 (s, 3H); MS (ES): m/z=302 (M+H).

### Example 13: Preparation of 1-methyl-6-(3′-methylbiphenyl-2-yloxy)-1H-pyrazolo[3,4-b]pyridine (CDD-1450

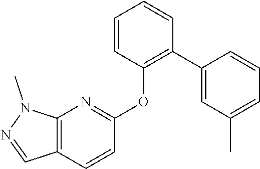 [SMILES: Cc1cccc(-c2ccccc2Oc2ccc3cnn(C)c3n2)c1]

### Step 1: Preparation of 6-chloro-1-methyl-1H-pyrazolo[3,4-b]pyridine

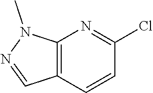

To a solution of 6-chloro-1H-pyrazolo[3,4-b]pyridine (200 mg, 1.3 mmol) in DMF (3 mL) was added sodium hydride (60% mineral oil dispersion, 78 mg, 1.9 mmol) at 0° C. and the mixture stirred at 0° C. for 0.5 h. Iodomethane (0.10 mL, 1.6 mmol) was added and the mixture stirred at room temperature for 2 h. Water (10 mL) was added and the mixture extracted with EtOAc (2×10 mL). The combined EtOAc layers were purified by combi-flash (EtOAc/petroleum ether: 0-50%) to give the expected product (160 mg, 73%) as a light yellow solid: MS (ES): m/z=168.1 (M+H).

### Step 2: Preparation of 1-methyl-6-(3′-methylbiphenyl-2-yloxy)-1H-pyrazolo[3,4-b]pyridine

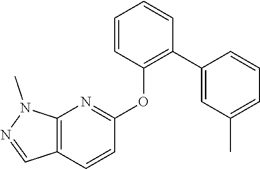 [SMILES: Cc1cccc(-c2ccccc2Oc2ccc3cnn(C)c3n2)c1]

A mixture of 6-chloro-1-methyl-1H-pyrazolo[3,4-b]pyridine (80 mg, 0.48 mmol), 3′-methylbiphenyl-2-ol (97 mg, 0.52 mmol) and K₂CO₃ (198 mg, 1.43 mmol) in DMF (3 mL) was heated at 130° C. overnight. After cooling the mixture was poured onto water (15 mL) and extracted with ethyl acetate (2×20 mL). The organic phase was concentrated under reduced pressure. The residue was purified by prep-HPLC to give the product (22 mg, 15%) as a yellow oil: ¹H NMR (400 MHz, DMSO-d6) δ 8.13 (d, J=8.6 Hz, 1H), 7.98 (s, 1H), 7.53-7.42 (m, 2H), 7.37 (td, J=7.4, 1.3 Hz, 1H), 7.29 (dd, J=8.0, 1.2 Hz, 1H), 7.26-7.14 (m, 3H), 7.04 (d, J=7.3 Hz, 1H), 6.70 (d, J=8.6 Hz, 1H), 3.76 (s, 3H), 2.23 (s, 3H); MS (ES): m/z=316.1 (M+H).

### Example 14: Preparation of 6-(3′-fluorobiphenyl-2-yloxy)-1-methyl-1H-pyrazolo[3,4-b]pyridine (CDD-1477

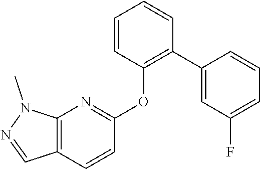 [SMILES: Cn1ncc2ccc(Oc3ccccc3-c3cccc(F)c3)nc21]

### Step 1: Preparation of 6-chloro-1-methyl-1H-pyrazolo[3,4-b]pyridine

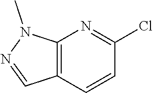

To a solution of 6-chloro-1-methyl-1H-pyrazolo[3,4-b]pyridine (200 mg, 1.30 mmol) in DMF (5 mL) was added sodium hydride (60% mineral oil dispersion, 78 mg, 1.95 mmol) at 0° C. under N₂. The mixture was stirred at 0° C. for 30 min, and then iodomethane (0.10 mL, 1.6 mmol) was added. The mixture was stirred at room temperature for 1.5 h. Cold water (5 mL) was dropped into the mixture which was extracted with ethyl acetate (2×10 mL). The organic phase was dried over Na₂SO₄ and concentrated in vacuo. The residue was purified by flash chromatography on silica gel (ethyl acetate in petroleum ether: 0-60%) to give the expected product (160 mg, 73%) as a yellow solid. The structure was confirmed by ¹H-NMR and 2D-NMR: ¹H NMR (400 MHz, DMSO-d6) δ 8.31 (d, J=8.3 Hz, 1H), 8.20 (s, 1H), 7.29 (d, J=8.3 Hz, 1H), 4.03 (s, 3H).

### Step 2: Preparation of 6-(3′-fluorobiphenyl-2-yloxy)-1-methyl-1H-pyrazolo[3,4-b]pyridine

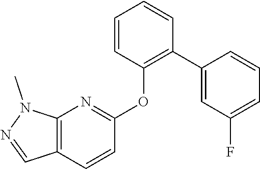 [SMILES: Cn1ncc2ccc(Oc3ccccc3-c3cccc(F)c3)nc21]

A mixture of 6-chloro-1-methyl-1H-pyrrolo[2,3-b]pyridine (200 mg, 1.20 mmol), 3′-fluorobiphenyl-2-ol (408 mg, 2.20 mmol), K₃PO₄ (509 mg, 2.40 mmol), cyclohexane-1,2-diamine (68 mg, 0.60 mmol), and CuI (23 mg, 0.10 mmol) in NMP (6 mL) was flushed with N₂ flow for 5 min, and then heated at 200° C. for 3 h. The mixture was cooled, concentrated and then purified by prep-HPLC to give the expected product (22 mg, 7%) as a yellow oil: ¹H NMR (400 MHz, DMSO-d6) δ: 8.15 (d, J=8.6 Hz, 1H), 7.99 (s, 1H), 7.56 (dd, J=7.6, 1.7 Hz, 1H), 7.50 (td, J=7.7, 1.7 Hz, 1H), 7.43-7.23 (m, 5H), 7.08 (td, J=8.1, 2.2 Hz, 1H), 6.75 (d, J=8.6 Hz, 1H), 3.76 (s, 3H); MS (ES): m/z=320.0 (M+H).

### Example 15: Preparation of 2-([1,1′-biphenyl]-2-yloxy)-7-methyl-7H-pyrrolo[2,3-d]pyrimidine (CDD-1306

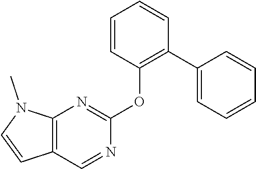 [SMILES: Cn1ccc2cnc(Oc3ccccc3-c3ccccc3)nc21]

### Step 1: 2-chloro-7-methyl-7H-pyrrolo[2,3-d]pyrimidine

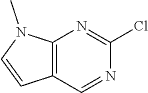

A solution of 2-chloro-7H-pyrrolo[2,3-d]pyrimidine (100 mg, 0.654 mmol), K₂CO₃ (271 mg, 1.96 mmol) and iodomethane (279 mg, 1.96 mmol) in DMF (2.0 mL) was stirred at 0° C. for 3 h. Water (30 mL) was added and the mixture extracted with DCM (3×50 mL). The combined DCM layers were washed with brine and concentrated in vacuo. The residue was purified by column chromatography to afford the desired material (83 mg, 76%) as a white solid: MS (ES) m/z=169 (M+H).

### Step 2: 2-([1,1′-biphenyl]-2-yloxy)-7-methyl-7H-pyrrolo[2,3-d]pyrimidine

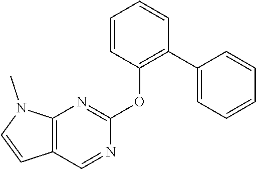 [SMILES: Cn1ccc2cnc(Oc3ccccc3-c3ccccc3)nc21]

A mixture of [1,1′-biphenyl]-2-ol (93 mg, 0.54 mmol), 2-chloro-7-methyl-7H-pyrrolo[2,3-d]pyrimidine (83 mg, 0.50 mmol), and K₂CO₃ (207 mg, 1.50 mmol) in DMF (2 mL) was heated at 110° C. overnight. After cooling water (50 mL) was added and the mixture extracted with DCM (3×100 mL). The combined DCM layers were washed with brine and concentrated in vacuo. The residue was purified by Prep-HPLC to afford the desired material (70 mg, 47%) as a white solid: ¹H NMR (400 MHz, DMSO-d6) δ: 8.67 (s, 1H), 7.50-7.37 (m, 1H), 7.37-7.27 (m, 1H), 7.27-7.17 (m, 1H), 6.51 (d, J=3.6 Hz, 1H), 3.64 (s, 1H); MS (ES) m/z=302 (M+H).

### Example 16: Preparation of 7-methyl-2-(2-(pyridin-3-yl)phenoxy)-7H-pyrrolo[2,3-d]pyrimidine (CDD-1399

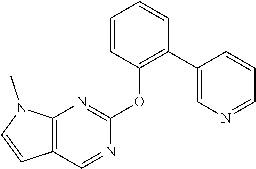 [SMILES: Cn1ccc2cnc(Oc3ccccc3-c3cccnc3)nc21]

### Step 1: Preparation of 2-chloro-7-methyl-7H-pyrrolo[2,3-d]pyrimidine

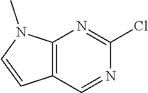

To a stirred solution of 2-chloro-7H-pyrrolo[2,3-d]pyrimidine (1.50 g, 9.77 mmoL) in acetonitrile (40 mL) at 0° C. was added sodium hydride (60% mineral oil dispersion, 470 mg, 11.7 mmol). The mixture was warmed to room temperature and stirred for 30 min. Iodomethane (3.00 g, 21.1 mmol) was added and the mixture stirred at room temperature for 2 h. The mixture was partitioned between water (40 mL) and ethyl acetate (3×30 mL). The combined organic phases were washed with brine (2×40 mL), dried over Na₂SO₄, and concentrated in vacuo. The residue was purified by chromatography on silica (ethyl acetate:petroleum ether 1:4) to give the expected product (1.3 g, 79%) as a white solid: MS (ES): m/z=168 (M+H).

### Step 2: Preparation of 7-methyl-2-(2-(pyridin-3-yl)phenoxy)-7H-pyrrolo[2,3-d]pyrimidine

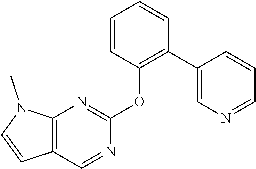 [SMILES: Cn1ccc2cnc(Oc3ccccc3-c3cccnc3)nc21]

To a solution of 2-chloro-7-methyl-7H-pyrrolo[2,3-d]pyrimidine (200 mg, 1.19 mmol) in N,N-dimethylformamide (6 mL) was added 2-(pyridin-3-yl)phenol (245 mg, 1.43 mmol) and potassium carbonate (294 mg, 3.58 mmol). The mixture was heated at 130° C. overnight. After cooling, the mixture was filtered and the filtrate purified by Prep-HPLC (A: water (10 mM NH₄HCO₃); B: acetonitrile: 20%-40% B) to give the expected product (104 mg, 29%) as a gray solid: ¹H NMR (400 MHz, DMSO-d6) δ: 8.67 (s, 1H), 8.64-8.62 (m, 1H), 8.43 (dd, J=4.8, 1.6 Hz, 1H), 7.88-7.83 (m, 1H), 7.56 (d, J=1.7 Hz, 1H), 7.49 (td, J=7.7, 1.7 Hz, 1H), 7.42-7.32 (m, 3H), 7.27 (dd, J=8.1, 1.1 Hz, 1H), 6.52 (d, J=3.6 Hz, 1H), 3.64 (s, 3H); MS (ES): m/z=303 (M+H).

### Example 17: Preparation of N-methyl-2′-(7-methyl-7H-pyrrolo[2,3-d]pyrimidin-2-yloxy)biphenyl-3-amine (CDD-1426

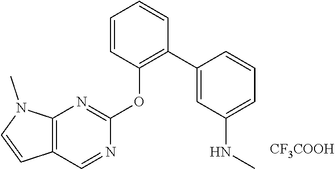

### Step 1: Preparation of tert-butyl 3-bromophenylcarbamate

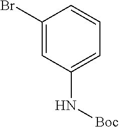 [SMILES: CC(C)(C)OC(=O)Nc1cccc(Br)c1]

Boc₂O (7.50 g, 34.9 mmol) was added to a solution of 3-bromoaniline (5.00 g, 29.1 mmol) in EtOH (50 mL) and the mixture heated at 38° C. for 3 h. The mixture was concentrated in vacuo. Petroleum ether (30 mL) was added and the resultant solid was filtered to give the desired product (7.0 g, 88%) as a light brown solid: ¹H NMR (400 MHz, DMSO-d6) δ: 9.57 (s, 1H), 7.76 (s, 1H), 7.40-7.32 (m, 1H), 7.21 (t, J=8.0 Hz, 1H), 7.14 (ddd, J=7.9, 1.9, 1.1 Hz, 1H), 1.47 (d, J=2.5 Hz, 9H).

### Step 2: Preparation of tert-butyl 3-bromophenyl(methyl)carbamate

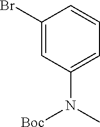 [SMILES: CN(C(=O)OC(C)(C)C)c1cccc(Br)c1]

Sodium hydride (60% mineral oil dispersion, 0.49 g, 12.1 mmol) was added portionwise to a solution of tert-butyl 3-bromophenylcarbamate (3.00 g, 11.0 mmol) in DMF (30 mL). After the evolution of gas had ceased, iodomethane (3.45 mL, 55.1 mmol) was added and the reaction mixture was stirred at 25° C. for 5 h. After addition of water (30 mL), the mixture was extracted with ethyl acetate (2×30 mL). The combined organic phase was dried over Na₂SO₄, filtered, and evaporated to give the product (2.9 g, 92%) as a brown oil: MS (ES): m/z=230.0 and 232.0 (M-55).

### Step 3: Preparation of tert-butyl 2′-hydroxybiphenyl-3-yl(methyl)carbamate

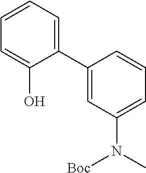 [SMILES: CN(C(=O)OC(C)(C)C)c1cccc(-c2ccccc2O)c1]

To a solution of tert-butyl 3-bromophenyl(methyl)carbamate (1.90 g, 6.64 mmol) and 2-hydroxyphenylboronic acid (1.37 g, 9.30 mmol) in dioxane/H₂O (10:1, 32 mL) was added K₂CO₃ (1.84 g, 113 mmol), and Pd(dppf)Cl₂ (0.49 g, 0.66 mmol) under N₂. The mixture was heated at 90° C. overnight. After cooling the mixture was filtered and the filtrate was concentrated in vacuo. The residue was purified by chromatography on silica gel (ethyl acetate in petroleum ether: 0-50%) to give the product (1.38 g, 69%) as a brown solid: MS (ES): m/z=322.0 (M+Na).

### Step 4: Preparation of tert-butyl methyl(2′-(7-methyl-7H-pyrrolo[2,3-d]pyrimidin-2-yloxy)biphenyl-3-yl)carbamate

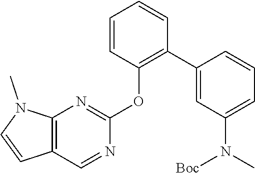 [SMILES: CN(C(=O)OC(C)(C)C)c1cccc(-c2ccccc2Oc2ncc3ccn(C)c3n2)c1]

To a solution of 2-chloro-7-methyl-7H-pyrrolo[2,3-d]pyrimidine (150 mg, 0.895 mmol) in DMF (5 mL) was added tert-butyl 2′-hydroxybiphenyl-3-yl(methyl)carbamate (295 mg, 0.984 mmol) and K₂CO₃ (371 mg, 2.69 mmol). The mixture was heated at 130° C. overnight. After cooling water (5 mL) was added and the mixture extracted with ethyl acetate (2×15 mL). The combined organic phase was concentrated under reduced pressure. The yellow oil was purified chromatography on silica gel (ethyl acetate in petroleum ether: 0-50%) to give the expected product (160 mg, 42%) as a yellow oil: MS (ES): m/z=431.2 (M+H).

### Step 5: Preparation of N-methyl-2′-(7-methyl-7H-pyrrolo[2,3-d]pyrimidin-2-yloxy)biphenyl-3-amine

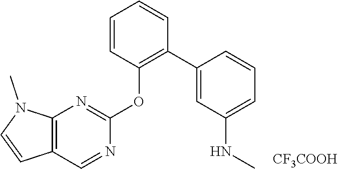 [SMILES: CNc1cccc(-c2ccccc2Oc2ncc3ccn(C)c3n2)c1 ; "CF3COOH"]

Trifluoroacetic acid (0.5 mL) was added to a solution of tert-butyl methyl (2′-(7-methyl-7H-pyrrolo[2,3-d]pyrimidin-2-yloxy)biphenyl-3-yl)carbamate (160 mg, 0.37 mmol) in DCM (2 mL). The mixture was stirred at room temperature for 1.5 h. The mixture was concentrated under reduced pressure. The residue was purified by prep-HPLC to get the expected product (112 mg, 68%) as a white solid: ¹H NMR (400 MHz, DMSO-d6) δ: 8.68 (s, 1H), 7.47-7.36 (m, 3H), 7.31 (dd, J=7.4, 6.4 Hz, 1H), 7.18 (d, J=7.6 Hz, 1H), 7.14-7.04 (m, 1H), 6.73 (s, 2H), 6.55 (dd, J=18.1, 5.0 Hz, 2H), 3.65 (s, 3H), 2.54 (s, 3H); MS (ES): m/z=331.2 (M+H).

### Example 18: Preparation of 6-(biphenyl-2-yloxy)-1-methyl-1H-pyrrolo[2,3-b]pyridine (CDD-1460

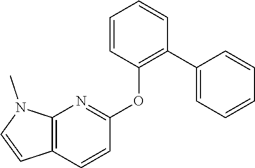 [SMILES: Cn1ccc2ccc(Oc3ccccc3-c3ccccc3)nc21]

A mixture of 6-chloro-1-methyl-1H-pyrrolo[2,3-b]pyridine (200 mg, 1.2 mmol), biphenyl-2-ol (408 mg, 2.4 mmol), K₃PO₄ (509 mg, 2.40 mmol), cyclohexane-1,2-diamine (68 mg, 0.60 mmol), and CuI (23 mg, 0.10 mmol) in NMP (6 mL) was flushed with N₂ for 5 min, and then heated at 200° C. for 3 h. The mixture was cooled and purified by prep-HPLC to give the expected product (27 mg, 8%) as a black oil: ¹H NMR (400 MHz, DMSO-d6) δ: 7.92 (d, J=8.3 Hz, 1H), 7.53-7.47 (m, 3H), 7.42-7.24 (m, 6H), 7.14 (dd, J=8.1, 1.1 Hz, 1H), 6.59 (d, J=8.3 Hz, 1H), 6.39 (d, J=3.4 Hz, 1H), 3.59 (s, 3H); MS (ES): m/z=301.0 (M+H).

### Example 19: Preparation of 6-(3′-fluorobiphenyl-2-yloxy)-1-methyl-1H-pyrrolo[2,3-b]pyridine (CDD-1479

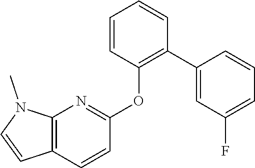 [SMILES: Cn1ccc2ccc(Oc3ccccc3-c3cccc(F)c3)nc21]

To a solution of 6-chloro-1-methyl-1H-pyrrolo[2,3-b]pyridine (160 mg, 0.96 mmol) in NMP (2 mL) was added 3′-fluorobiphenyl-2-ol (363 mg, 1.93 mmol), K₃PO₄ (409 mg, 1.93 mmol), CuI (18 mg, 0.10 mmol), and cyclohexane-1,2-diamine (55 mg, 0.48 mmol). The mixture was evacuated and re-charged with N₂ three times, heated at 150° C. for 30 min, and at 180° C. for 3.5 h. After cooling, water (20 mL) was added and the mixture extracted with EtOAc (2×20 mL). The combined EtOAc layers were concentrated and the residue was purified by prep-HPLC to afford the expected product (22 mg, 7%) as a yellow oil: ¹H NMR (400 MHz, DMSO-d6) δ: 7.92-7.94 (dd, J=2.3, 0.8 Hz, 1H), 7.5 (dd, J=4.8, 1.6 Hz, 1H), 7.5-7.1 (s, 6H), 7.1-6.6 (m, 2H), 6.6 (m, 1H), 6.39-6.4 (m, 1H), 3.59 (s, 3H); MS (ES) m/z=319 (M+H).

### Example 20: Preparation of 1-methyl-6-(3′-methylbiphenyl-2-yloxy)-1H-pyrrolo[2,3-b]pyridine (CDD-1478

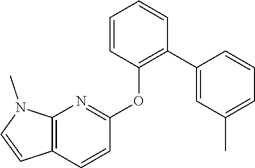 [SMILES: Cc1cccc(-c2ccccc2Oc2ccc3ccn(C)c3n2)c1]

### Step 1: Preparation of 3′-methylbiphenyl-2-ol

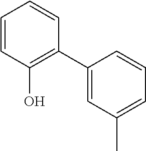

A mixture of 3-bromotoluene (1.00 g, 5.88 mmol), K₂CO₃ (1.62 g, 11.8 mmol), 3-hydroxyphenylboronic acid (1.14 g, 8.24 mmol), and PdCl₂(PPh₃)₄ (430 mg, 0.590 mmol) in 1-120 (1.5 ml) and dioxane (15 mL) was flushed with N₂, and then heated at 90° C. overnight. After cooling the mixture was diluted with water and extracted EtOAc. The combined EtOAc layers were concentrated in vacuo. The residue was purified by chromatography on silica gel to afford the expected product (0.80 g, 74%) as a yellow oil: MS (ES): m/z=185 (M+H).

### Step 2: Preparation of 1-methyl-6-(3′-methylbiphenyl-2-yloxy)-1H-pyrrolo[2,3-b]pyridine

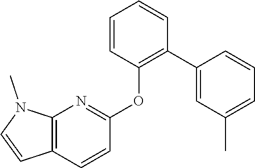 [SMILES: Cc1cccc(-c2ccccc2Oc2ccc3ccn(C)c3n2)c1]

To a solution of 6-chloro-1-methyl-1H-pyrrolo[2,3-b]pyridine (150 mg, 0.900 mmol) in NMP (2 mL) was added 3′-methylbiphenyl-2-ol (329 mg, 1.81 mmol), K₃PO₄ (380 mg, 1.81 mmol), CuI (17 mg, 0.09 mmol), and cyclohexane-1,2-diamine (51 mg, 0.45 mmol). The mixture was evacuated and re-changed with N₂ three times, heated at 150° C. for 30 min, and at 180° C. for 3.5 h. After cooling, water (20 mL) was added and the mixture extracted with EtOAc (2×20 mL). The combined EtOAc layers were concentrated and the residue was purified by prep-HPLC to afford the expected product (71 mg, 11%) as a yellow oil: ¹H NMR (400 MHz, DMSO-d6) δ: 7.91-7.93 (d, J=9.6 Hz, 1H), 7.09-7.49 (m, 9H), 6.57-6.59 (d, J=9.6 Hz, 1H), 6.39-6.40 (d, J=9.6 Hz, 1H), 3.6 (s, 3H), 2.27 (s, 3H); MS (ES): m/z=315 (M+H).

### Example 21: Preparation of 5-([1,1′-biphenyl]-2-yloxy)-1,3-dimethyl-1H-pyrazolo[4,3-d]pyrimidine (CDD-1435

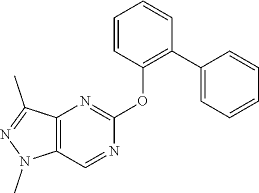 [SMILES: Cc1nn(C)c2cnc(Oc3ccccc3-c3ccccc3)nc12]

### Step 1: Preparation of 1,3-dimethyl-4-nitro-1H-pyrazole-5-carboxylic acid

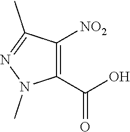

To fuming nitric acid (1.26 mL, 30.0 mmol) at 0° C. was slowly added fuming sulfuric acid (9.76 mL, 105 mmol) dropwise over 30 minutes. At this time 1,3-dimethyl-1H-pyrazole-5-carboxylic acid (2.1 g, 15.0 mmol) was added portion-wise, maintaining the internal temperature below 60° C. The reaction mixture was stirred at 60° C. for 4 h and then cooled to room temperature. The reaction mixture was poured onto ice. When the ice melted, the reaction mixture was extracted with EtOAc (3×100 mL). The organic layers were combined, washed with water and brine, dried over Na₂SO₄, filtered, and evaporated in vacuo to give the desired compound (1.75 g. 63%) as a white solid: MS (ES): m/z=186 (M+H).

### Step 2: Preparation of methyl 1,3-dimethyl-4-nitro-1H-pyrazole-5-carboxylate

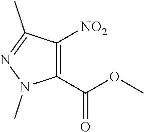

A solution of 1,3-dimethyl-4-nitro-1H-pyrazole-5-carboxylic acid (3.20 g, 17.3 mmol), K₂CO₃ (9.56 g, 69.2 mmol), and iodomethane (7.37 g, 51.9 mmol) in DMF (30 mL) was stirred at 30° C. overnight. Water (100 mL) was added and the mixture extracted with DCM (3×100 mL). The combined DCM layers were washed with brine and concentrated in vacuo. The residue was purified by column chromatography to afford the desired material (2.42 g, 71%) as a white solid: MS (ES): m/z=200 (M+H).

### Step 3: Preparation of methyl 4-amino-1,3-dimethyl-1H-pyrazole-5-carboxylate

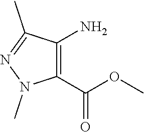

A mixture of methyl 1,3-dimethyl-4-nitro-1H-pyrazole-5-carboxylate (2.42 g, 12.2 mmol) and Raney-Ni (240 mg, 10% w) in MeOH (30 mL) was stirred at room temperature for 6 h under a H₂ atmosphere and then filtered through Celite. The filtrate was concentrated in vacuo to afford the desired material (1.89 g, 92%) as a yellow solid: MS (ES): m/z=170 (M+H).

### Step 4: Preparation of 1,3-dimethyl-5-thioxo-5,6-dihydro-1H-pyrazolo[4,3-d]pyrimidin-7(4H)-one

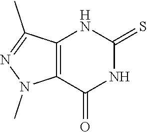

Benzoyl isothiocyanate (109 mg, 0.67 mmol) was added to a solution of methyl 4-amino-1,3-dimethyl-1H-pyrazole-5-carboxylate (103 mg, 0.610 mmol) in acetone (5 mL) and the mixture stirred for 1 h. The mixture was partitioned between EtOAc and brine. The organic layer was dried over MgSO₄ and evaporated in vacuo. The resulting crude material was dissolved in acetone (6 mL), MeOH (6 mL) and water (1.5 mL). Potassium carbonate (165 mg, 1.22 mmol) was added and the mixture heated at reflux for 2 h. The reaction mixture was cooled, concentrated and purified by Prep-HPLC to afford the desired material (85 mg, 71%) as a white solid: MS (ES): m/z=197 (M+H).

### Step 5: Preparation of 1,3-dimethyl-5-(methylthio)-1H-pyrazolo[4,3-d]pyrimidin-7(6H)-one

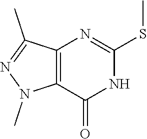

A mixture of 1,3-dimethyl-5-thioxo-5,6-dihydro-1H-pyrazolo[4,3-d]pyrimidin-7(4H)-one (1.1 g, 5.6 mmol), K₂CO₃ (2.32 g, 16.8 mmol), and iodomethane (877 mg, 6.17 mmol) in DMF (15 mL) was stirred at room temperature for 1 h. Water (100 mL) was added and the mixture extracted with DCM (3×100 mL). The combined DCM layers were washed with brine and concentrated in vacuo. The residue was purified by silica gel chromatography to afford the desired material (982 mg, 84%) as a white solid: MS (ES): m/z=211 (M+H).

### Step 6: Preparation of 7-chloro-1,3-dimethyl-5-(methylthio)-1H-pyrazolo[4,3-d]pyrimidine

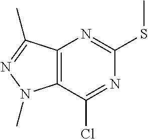

To a solution of 1,3-dimethyl-5-(methylthio)-1H-pyrazolo[4,3-d]pyrimidin-7(6H)-one (400 mg, 1.9 mmol) in POCl₃ (10 mL) was added DMF (0.1 mL) and pyridine (0.1 mL). The mixture was heated at 100° C. for 2 h. After cooling the mixture was concentrated in vacuo. The crude residue was neutralized with saturated aqueous NaHCO₃ solution and extracted with EtOAc (3×50 mL). The combined organic layers were dried over anhydrous Na₂SO₄ and concentrated in vacuo. The residue was purified by silica gel chromatography to afford the desired material (350 mg, 81%) as an off-white solid: MS (ES): m/z=229 (M+H).

### Step 7: Preparation of 1,3-dimethyl-5-(methylthio)-1H-pyrazolo[4,3-d]pyrimidine

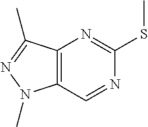

A mixture of 7-chloro-1,3-dimethyl-5-(methylthio)-1H-pyrazolo[4,3-d]pyrimidine (200 mg, 0.873 mmol) and Pd/C (20 mg, 10% w) in isopropanol (5 mL) was stirred at room temperature for 6 h under an H₂ atmosphere. The mixture was filtered through Celite and the filtrate concentrated in vacuo. The residue was purified by column chromatography to afford the desired material (105 mg, 62%) as an off-white solid: MS (ES): m/z=195 (M+H).

### Step 8: Preparation of 1,3-dimethyl-5-(methylsulfonyl)-1H-pyrazolo[4,3-d]pyrimidine

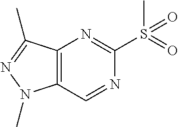

To a solution of 1,3-dimethyl-5-(methylthio)-1H-pyrazolo[43-d]pyrimidine (105 mg, 0.54 mmol) in dichloromethane (5 mL) was added 3-chloroperbenzoic acid (186 mg, 1.08 mmol) in several portions at 0-5° C. The reaction mixture was stirred overnight at room temperature and then quenched with saturated NaHCO₃ solution. The organic layer was separated, dried over sodium sulfate, filtered, and evaporated. The residue was purified by silica gel chromotography to afford the desired material as an off-white solid (80 mg, 66%): MS (ES): m/z=227 (M+H).

### Step 9: Preparation of 5-([1,1′-biphenyl]-2-yloxy)-1,3-dimethyl-1H-pyrazolo[4,3-d]pyrimidine

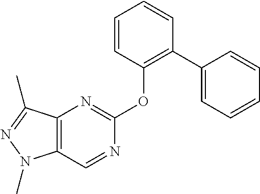 [SMILES: Cc1nn(C)c2cnc(Oc3ccccc3-c3ccccc3)nc12]

A mixture of 1,3-dimethyl-5-(methylsulfonyl)-1H-pyrazolo[4,3-d]pyrimidine (80 mg, 0.35 mmol), [1,1′-biphenyl]-2-ol (90 mg, 0.53 mmol), and K₂CO₃ (147 mg, 1.06 mmol) in DMF (2 mL) was heated at 130° C. overnight. After cooling, water (50 mL) was added and the mixture extracted with DCM (3×100 mL). The combined DCM layers were washed with brine and concentrated in vacuo. The residue was purified by Prep-HPLC to afford the desired material (25 mg, 22%) as an off-white solid: ¹H NMR (400 MHz, DMSO-d6) δ: 9.16 (s, 1H), 7.54-7.19 (m, 9H), 4.04 (s, 3H), 2.34 (d, J=11.5 Hz, 3H); MS (ES): m/z=317 (M+H).

### Example 22: Preparation of 2-(biphenyl-2-yloxy)pyrido[4,3-d]pyrimidine (CDD-1449

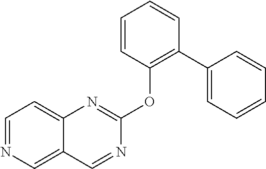 [SMILES: c1ccc(-c2ccccc2Oc2ncc3cnccc3n2)cc1]

### Step 1: Preparation of ethyl 2-(ethoxymethylene)-3-oxobutanoate

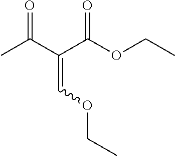

Ethyl 3-oxobutanoate (19.5 mL, 205 mmol) was added to a solution of acetic anhydride (13.0 mL, 103 mmol) in triethoxymethane (16.9 mL, 103 mmol). The mixture was heated at 120° C. for 2 hours under N₂. Upon completion the reaction mixture was cooled to room temperature and evaporated in vacuum. The residue was distilled to give the desired product (bp 148° C., 15 g, 78%) as an orange yellow liquid, which was used directly in next step.

### Step 2: Preparation of ethyl 4-methyl-2-(methylthio)pyrimidine-5-carboxylate

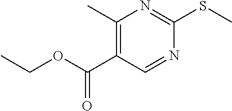

Triethylamine (11.6 mL, 83.8 mmol) was added dropwise to a solution of ethyl 2-(ethoxymethylene)-3-oxobutanoate (15.0 g, 80.6 mmol) and 2-methyl-2-thiopseudourea sulfate (26.9 g, 96.7 mmol) in EtOH (30 mL). The mixture was heated at 100° C. for 2 h. Ice water (70 mL) was added at 0° C. and the mixture stirred at room temperature overnight. The suspension was filtered, the solid washed with water, and dried under reduced pressure to give the title compound (10 g, 59%) as an off-white solid: ¹H NMR (400 MHz, CDCl₃) δ: 8.95 (s, 1H), 4.38 (q, J=7.1 Hz, 2H), 2.77 (s, 3H), 2.60 (s, 3H), 1.40 (t, J=7.1 Hz, 3H).

### Step 3: Preparation of ethyl 4-(2-(dimethylamino)vinyl)-2-(methylthio)pyrimidine-5-carboxylate

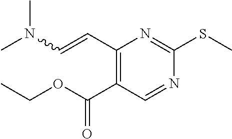 [SMILES: CCOC(=O)c1cnc(SC)nc1C=CN(C)C]

To a solution of ethyl 4-methyl-2-(methylthio)pyrimidine-5-carboxylate (10 g, 47 mmol) in DMF (50 mL) was added 1,1-dimethoxy-N,N-dimethylmethanamine (12.7 mL, 94.2 mmol). The mixture was heated at 130° C. for 2 h. The mixture was cooled and added to ice-water (150 mL). The yellow precipitate was filtered, washed with water, and dried for 14 h at 40° C. to give the expected product (14 g, 84%) as a yellow solid: MS (ES): m/z=268.1 (M+H).

### Step 4: Preparation of 2-(methylthio)pyrido[4,3-d]pyrimidin-5(6H)-one

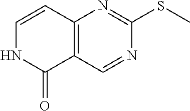

A mixture of ethyl 4-(2-(dimethylamino)vinyl)-2-(methylthio)pyrimidine-5-carboxylate (4.00 g, 15.0 mmol), ammonium acetate (3.46 g, 44.9 mmol), and ammonium hydroxide (4 mL) in EtOH (10 mL) was heated at 100° C. for 24 h. The mixture was cooled to room temperature, filtered, washed with EtOH (10 mL), and dried under reduced pressure to give the desired product (1.8 g, 62%) as a yellow solid: MS (ES): m/z=193.2.

### Step 5: Preparation of 5-chloro-2-(methylthio)pyrido[4,3-d]pyrimidine

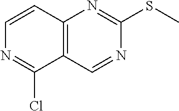

A mixture of 2-(methylthio)pyrido[4,3-d]pyrimidin-5(6H)-one (1.80 g, 9.32 mmol) in POCl₃ (2.86 mL) was heated at 105° C. for 2 h. After the mixture was cooled to room temperature, it was poured onto ice-water, neutralized with saturated NaHCO₃ solution, and extracted with ethyl acetate (2×30 mL). The combined EtOAc layers were dried and concentrated to obtain the desired product as a brown solid (1.8 g, 91%): MS (ES): m/z=211.9 (M+H).

### Step 6: Preparation of 2-(methylthio)pyrido[4,3-d]pyrimidine

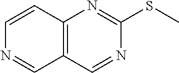

A suspension of 5-chloro-2-(methylthio)pyrido[4,3-d]pyrimidine (0.60 g, 2.8 mmol), ammonium formate (1.79 g, 28.3 mmol), and Pd/C (10%, 0.6 g) in MeOH (9 mL) in sealed tube was heated at 100° C. overnight. After cooling, the mixture was filtered and the filtrate was concentrated. The residue was purified by chromatography on silica gel (ethyl acetate in petroleum ether: 0-75%) to give the product (0.32 g, 64%) as a yellow solid: ¹H NMR (400 MHz, CDCl₃) δ: 9.25 (d, J=8.1, 0.8 Hz, 2H), 8.84 (d, J=6.0 Hz, 1H), 7.67 (d, J=6.0 Hz, 1H), 2.70 (s, 3H).

### Step 7: Preparation of 2-(biphenyl-2-yloxy)pyrido[4,3-d]pyrimidine

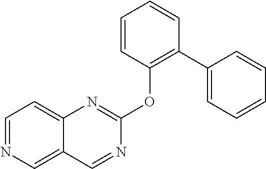 [SMILES: c1ccc(-c2ccccc2Oc2ncc3cnccc3n2)cc1]

Sulfuryl dichloride (0.65 mL, 5.6 mmol) was added to a mixture of 2-(methylthio)pyrido[4,3-d]pyrimidine (0.10 g, 0.56 mmol) in MeCN (2 mL) and DCM (2.5 mL) at 0° C. 2-hydroxy biphenyl was added. After the mixture was stirred for 1.5 h at 0° C., the precipitate was filtered and washed with MeCN to give a yellow solid. DMF (2 mL) was added to the solid followed by biphenyl-2-ol (209 mg, 1.13 mmol) and K₂CO₃ (234 mg, 1.89 mmol). The mixture was heated at 130° C. overnight and was then allowed to cool. The residue was concentrated and purified by prep-HPLC to give the desired product (1.3 mg, 1%) as a white solid: ¹H NMR (400 MHz, DMSO-d6) δ: 9.65 (s, 1H), 9.40 (s, 1H), 8.78 (d, J=5.9 Hz, 1H), 7.60 (d, J=5.4 Hz, 1H), 7.56-7.35 (m, 6H), 7.25 (d, J=22.3, 6.7 Hz, 3H); MS (ES): m/z=300.0 (M+H).

### Example 23: Preparation of N-methyl-2′-(pyrido[4,3-d]pyrimidin-2-yloxy)biphenyl-3-amine (CDD-001506

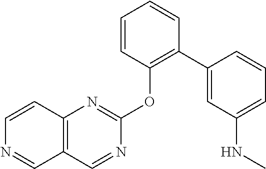 [SMILES: CNc1cccc(-c2ccccc2Oc2ncc3cnccc3n2)c1]

### Step 1: Preparation of tert-butyl methyl(2′-(pyrido[4,3-d]pyrimidin-2-yloxy)biphenyl-3-yl)carbamate

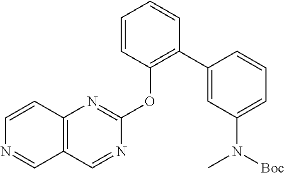 [SMILES: CN(C(=O)OC(C)(C)C)c1cccc(-c2ccccc2Oc2ncc3cnccc3n2)c1]

Sulfuryl dichloride (0.60 mL, 11 mmol) in DCM (1.5 mL) was added to a solution of 2-(methylthio)pyrido[4,3-d]pyrimidine (0.20 g, 1.1 mmol) in MeCN (2.5 mL) and DCM (3 mL) at 0° C. The mixture was stirred for 2 h at 0° C. and the solvent was blown away by N₂. NMP (3 mL) was added to the residue followed by tert-butyl 2′-hydroxybiphenyl-3-yl(methyl)carbamate (325 mg, 1.09 mmol) and DIPEA (1.18 g, 9.10 mmol). The mixture was heated at 60° C. overnight. After cooling the mixture was partitioned between EtOAc and water. The organic phase was concentrated and the residue purified by prep-TLC to give the desired product (150 mg, 31%) as a yellow oil: MS (ES): m/z=429.1 (M+H).

### Step 2: Preparation of N-methyl-2′-(pyrido[4,3-d]pyrimidin-2-yloxy)biphenyl-3-amine

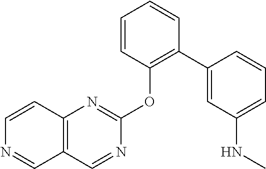 [SMILES: CNc1cccc(-c2ccccc2Oc2ncc3cnccc3n2)c1]

TFA (0.5 mL) was added to a solution of tert-butyl methyl(2′-(pyrido[4,3-d]pyrimidin-2-yloxy)biphenyl-3-yl)carbamate (150 mg, 0.35 mmol) in DCM (2 mL) and the mixture stirred at room temperature for 2 h. The mixture was concentrated and the residue was purified by prep-HPLC to give the desired product (11 mg, 9%) as a white solid: ¹H NMR (400 MHz, DMSO-d6) δ 9.68 (s, 1H), 9.42 (s, 1H), 8.78 (d, J=6.0 Hz, 1H), 7.62 (d, J=6.0 Hz, 1H), 7.51-7.42 (m, 2H), 7.43-7.31 (m, 2H), 6.97 (t, J=7.8 Hz, 1H), 6.54 (d, J=7.7 Hz, 1H), 6.49 (s, 1H), 6.38 (dd, J=8.1, 1.6 Hz, 1H), 5.57 (d, J=5.0 Hz, 1H), 2.45 (d, J=5.1 Hz, 3H); MS (ES): m/z=329.0 (M+H).

### Example 24: Preparation of 2-(biphenyl-2-yloxy)-1,6-naphthyridine (CDD-1487

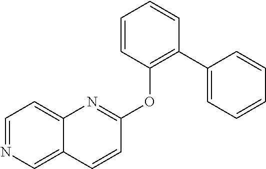 [SMILES: c1ccc(-c2ccccc2Oc2ccc3cnccc3n2)cc1]

To a solution of 2-chloro-1,6-naphthyridine (100 mg, 0.61 mmol) in DMF (1 mL) was added K₂CO₃ (168 mg, 1.22 mmol), CuI (11 mg, 0.06 mmol), biphenyl-2-ol (207 mg, 1.22 mmol), and cyclohexane-1,2-diamine (35 mg, 0.31 mmol). The mixture was flushed with N₂ three times, and then heated at 130° C. for 15 h. After cooling the mixture was partitioned between water (20 mL) and EtOAc (2×25 mL). The combined EtOAc layers were concentrated in vacuo and the residue was purified by prep-HPLC to afford 2-(biphenyl-2-yloxy)-1,6-naphthyridine (37 mg, 21%) as a yellow oil: ¹H NMR (400 MHz, DMSO-d6) δ 9.21 (s, 1H), 8.56 (s, 1H), 8.48 (d, J=8.7 Hz, 1H), 7.51 (qd, J=7.7, 1.8 Hz, 3H), 7.40 (tdd, J=19.0, 7.7, 1.2 Hz, 4H), 7.28 (dt, J=7.6, 2.9 Hz, 3H), 7.24-7.19 (m, 1H); MS (ES) m/z=299 (M+H).

### Example 25: Preparation of 2-(2-(pyridin-3-yl)phenoxy)-1,6-naphthyridine (CDD-1497

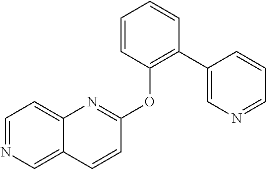 [SMILES: c1cncc(-c2ccccc2Oc2ccc3cnccc3n2)c1]

To a mixture of 2-chloro-1,6-naphthyridine (45 mg, 0.28 mmol) in DMF (3 mL) was added K₂CO₃ (78 mg, 0.56 mmol), CuI (6.0 mg, 0.028 mmol), 2-(pyridin-3-yl)phenol (96 mg, 0.56 mmol), and cyclohexane-1,2-diamine (17 mg, 0.14 mmol). The mixture was evacuated and re-charged with N₂ three times. The mixture was heated at 130° C. for 15 h. After cooling water (15 mL) was added and the mixture extracted with EtOAc (2×20 mL). The combined EtAOc layers were concentrated in vacuo. The residue was purified by prep-HPLC to afford 2-(2-(pyridin-3-yl)phenoxy)-1,6-naphthyridine (12 mg, 14%) as a yellow oil: ¹H NMR (400 MHz, DMSO-d6) δ 9.22 (s, 1H), 8.63-8.56 (m, 2H), 8.50 (d, J=9.0 Hz, 1H), 8.41 (d, J 4.7 Hz, 1H), 7.82 (d, J=8.1 Hz, 1H), 7.58 (dd, J=15.0, 7.6 Hz, 2H), 7.53-7.39 (m, 3H), 7.31 (dd, J=8.3, 3.51 Hz, 2H); MS (ES): m/z=300 (M+H).

### Example 26: N-([1,1′-biphenyl]-2-yl)-1,6-naphthyridin-2-amine (SLUPP-1101

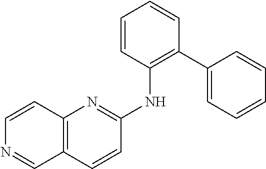 [SMILES: c1ccc(-c2ccccc2Nc2ccc3cnccc3n2)cc1]

To a mixture of 2-chloro-1,6-naphthyridine (100 mg, 0.61 mmol) and 2-aminobiphenyl (123 mg, 0.73 mmol) in toluene (2 mL) was added NaOtBu (117 mg, 1.22 mmol), Pd₂(dba)₃ (63 mg, 0.06 mmol), and BINAP (76 mg, 0.12 mmol). The mixture was flushed with argon three times, and then heated at 110° C. for 2 h. After cooling the mixture was diluted with water (10 mL) and extracted with EtOAc (2×25 mL). The combined organic layers were washed with brine, dried over anhyd Na₂SO₄ and concentrated in vacuo. The residue was purified on silica gel (hexane/EtOAc) to afford the desired product as a pale solid (98 mg, 54%); ¹H NMR (400 MHz, DMSO-d₆) δ ppm 6.90 (d, J=9.05 Hz, 1H) 7.21-7.26 (m, 1H) 7.27-7.36 (m, 4H) 7.38-7.45 (m, 4H) 7.67 (d, J=7.09 Hz, 1H) 8.02 (d, J=8.80 Hz, 1H) 8.38 (d, J=5.87 Hz, 1H) 8.87 (d, J=0.49 Hz, 1H) 9.09 (s, 1H). MS (ES): m/z=298.2 (M+H).

### Example 27: N-([1,1′-biphenyl]-2-yl)-N,N-dimethyl-1,7-naphthyridin-2-aminium iodide (SLUPP-1102

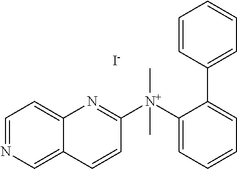 [SMILES: C[N+](C)(c1ccc2cnccc2n1)c1ccccc1-c1ccccc1 ; [I-]]

To a mixture of N-([1,1′-biphenyl]-2-yl)-1,7-naphthyridin-2-amine (39 mg, 0.13 mmol) and K₂CO₃ (34 mg, 0.26 mmol) in acetone (1 mL) was added CH₃I (114 mg, 0.8 mmol). The mixture was stirred at room temperature for 1 h. The reaction was concentrated in vacuo, water was added and was extracted with EtOAc (3×). The combined organic layers were washed with brine, dried over anhyd Na₂SO₄ and concentrated in vacuo. The residue was purified on silica gel (hexane/EtOAc) to afford the desired product as a pale solid (22 mg, 52%); ¹H NMR (400 MHz, CHLOROFORM-d₆) δ ppm 4.38 (s, 3H) 7.05 (d, J=8.80 Hz, 1H) 7.18-7.33 (m, 5H) 7.45-7.68 (m, 4H) 8.09 (d, J=8.07 Hz, 1H) 8.25 (d, J=6.36 Hz, 1H) 8.46 (d, J=6.36 Hz, 1H) 9.43 (br. s., 1H). MS (ES): m/z=326.2 (M+).

### Example 28: Preparation of N-benzyl-2-((7-methoxy-4-(trifluoromethyl)quinolin-2-yl)thio) (SLUPP-975

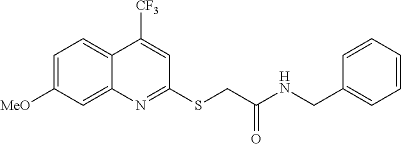 [SMILES: COc1ccc2c(C(F)(F)F)cc(SCC(=O)NCc3ccccc3)nc2c1]

### Step 1: Preparation of methyl 2-((7-methoxy-4-(trifluoromethyl)quinolin-2-yl)thio)acetate

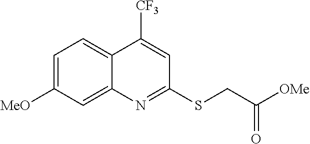 [SMILES: COC(=O)CSc1cc(C(F)(F)F)c2ccc(OC)cc2n1]

A mixture of 2-chloro-7-methoxy-4-(trifluoromethyl)quinoline (400 mg, 1.53 mmol), K₂CO₃ (634 mg, 4.59 mmol), and methyl thioglycolate (194.7 mg, 1.83 mmol) in acetonitrile (10 ml) was stirred overnight at room temperature (rt). The reaction was concentrated in vacuo, water was added and was extracted with EtOAc (3×). The combined organic layers were washed with brine, dried over anhyd Na₂SO₄ and concentrated in vacuo. The residue was purified on silica gel (hexane/EtOAc) to afford the desired product as a white solid (382 mg, 76%); ¹H NMR (400 MHz, DMSO-d6) δ ppm: 3.70 (s, 3H), 3.95 (s, 3H), 4.23 (s, 2H), 7.30 (d, J=2.69 Hz, 1H), 7.35 (dd, J=9.29, 2.69 Hz, 1H), 7.76 (s, 1H), 7.89 (dd, J=9.17, 2.08 Hz, 1H); MS (ES) m/z=332.1 (M+H).

### Step 2: Preparation of 2-((7-methoxy-4-(trifluoromethyl)quinolin-2-yl)thio)acetic acid

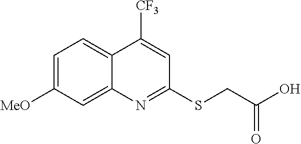 [SMILES: COc1ccc2c(C(F)(F)F)cc(SCC(=O)O)nc2c1]

To a mixture of methyl 2-((7-methoxy-4-(trifluoromethyl)quinolin-2-yl)thio)acetate (380 mg, 1.15 mmol) in THF (8 mL) was added LiOH solution in minimum H₂O and stirred for 1 hr at rt. The reaction was concentrated in vacuo, acidified with 1N HCl and extracted with EtOAc (3×). The combined organic layers were washed with brine, dried over anhyd Na₂SO₄ and concentrated in vacuo to afford the desired product as a white solid (319 mg, 87%): ¹H NMR (400 MHz, CHLOROFORM-d) 6 ppm: 1.58 (br. s., 1H), 3.96 (s, 2H), 3.99 (s, 3H), 7.29-7.33 (m, 2H), 7.54 (s, 1H), 8.00 (dd, J=9.78, 1.96 Hz, 1H); MS (ES): m/z=318.0 (M+H).

### Step 3: Preparation of N-benzyl-2-((7-methoxy-4-(trifluoromethyl)quinolin-2-yl)thio) acetamide (SLUPP-975

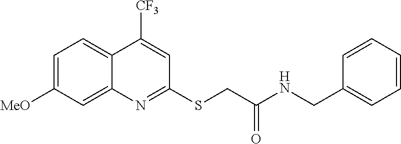 [SMILES: COc1ccc2c(C(F)(F)F)cc(SCC(=O)NCc3ccccc3)nc2c1]

A mixture of 2-((7-methoxy-4-(trifluoromethyl)quinolin-2-yl)thio)acetic acid (75 mg, 0.24 mmol), TBTU (76 mg, 0.24 mmol), and Hunig's base (0.12 ml, 0.71 mmol) in DMF (2 mL) was stirred under argon for 20 min at rt. Phenylmethanamine was added and stirred overnight. Mixture was quenched with water was and extracted with EtOAc (3×). The combined organic layers were washed with NaHCO₃ and brine, dried over anhydrous Na₂SO₄ and concentrated in vacuo. The residue was purified on a 50g C18 reversed-phase column (acetonitrile/water) to afford the desired product as a white solid (77 mg, 79%): ¹H NMR (400 MHz, DMSO-d6) δ ppm: 3.90 (s, 3H), 4.13 (s, 2H), 4.31 (d, J=6.11 Hz, 2H), 7.15-7.22 (m, 5H), 7.34-7.37 (m, 1H), 7.37-7.39 (m, 1H), 7.73 (s, 1H), 7.88-7.91 (m, 1H), 8.74 (t, J=5.75 Hz, 1H); MS (ES): m/z=407.1 (M+H).

Following the procedures similar to those outlined for Examples 1 through 28 the following compounds were prepared as shown in Table 2.

TABLE 2
Additional Compound Examples

| Ex. No. | | |
|---|---|---|
| CDD# | Name/Structure | Spectral Data |
| 29 | 2-(2-(benzyloxy)phenoxy)-1,7-naphthyridine | ¹H NMR (400 MHz, DMSO-d₆) δ ppm |
| PP- | | 5.06 (s, 2 H) 6.94 − 6.98 (m, 2 H) 7.03 − |
| 1199 | 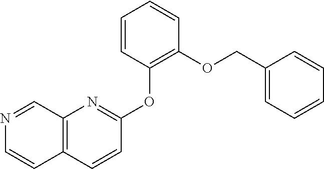 [SMILES: c1ccc(COc2ccccc2Oc2ccc3ccncc3n2)cc1] | 7.10 (m, 3 H) 7.11 − 7.17 (m, 1 H) 7.22 − 7.30 (m, 2 H) 7.32 (dd, J = 7.82, 1.47 Hz, 1 H) 7.62 (d, J = 8.80 Hz, 1 H) 8.00 (d, J = 5.14 Hz, 1 H) 8.50 (d, J = 8.56 Hz, 1 H) 8.55 (d, J = 5.62 Hz, 1 H) 9.07 (s, 1 H). MS (ES): m/z = 329.1 (M + H) |
| 30 | 2-((4′-bromo-[1,1′-biphenyl]-2-yl)oxy)-1,7-naphthyridine | ¹H NMR (400 MHz, DMSO-d₆) δ ppm |
| PP- | | 7.36 − 7.40 (m, 3 H) 7.40 − 7.45 (m, 1 H) |
| 1198 | 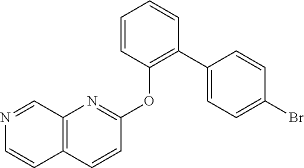 [SMILES: Brc1ccc(-c2ccccc2Oc2ccc3ccncc3n2)cc1] | 7.47 − 7.51 (m, 3 H) 7.51 − 7.56 (m, 2 H) 7.93 (d, J = 5.14 Hz, 1 H) 8.45 (d, J = 9.05 Hz, 1 H) 8.53 (d, J = 5.62 Hz, 1 H) 9.02 (s, 1 H). MS (ES): m/z = 379.0 (M + H) |
| 31 | 2-(2-(pyridin-3-yl)-5-(trifluoromethyl)phenoxy)-1,7- | ¹H NMR (400 MHz, DMSO-d₆) δ ppm |
| PP- | naphthyridine | 7.46 (dd, J = 7.82, 4.89 Hz, 1 H) 7.56 (d, |
| 1197 | | J = 9.05 Hz, 1 H) 7.84 − 7.90 (m, 2 H) 7.93 − |
| | 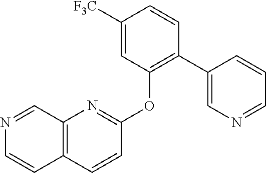 [SMILES: FC(F)(F)c1ccc(-c2cccnc2)c(Oc2ccc3ccncc3n2)c1] | 7.97 (m, 2 H) 8.04 (dt, J = 8.07, 1.96 Hz, 1 H) 8.48 (d, J = 8.80 Hz, 1 H) 8.52 (dd, J = 4.89, 1.47 Hz, 1 H) 8.55 (d, J = 5.62 Hz, 1 H) 8.75 (d, J = 1.47 Hz, 2 H) 9.04 (s, 1 H). MS (ES): m/z = 368.1 (M + H) |
| 32 | 2-(2-(benzyloxy)phenoxy)-1,6-naphthyridine | ¹H NMR (400 MHz, DMSO-d₆) δ ppm |
| PP- | | 5.07 (s, 2 H) 6.97 − 7.01 (m, 2 H) 7.03 − |
| 1196 | 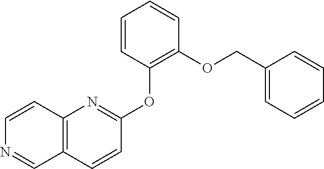 [SMILES: c1ccc(COc2ccccc2Oc2ccc3cnccc3n2)cc1] | 7.18 (m, 3 H) 7.22 − 7.29 (m, 2 H) 7.29 − 7.34 (m, 1 H) 7.54 (d, J = 8.80 Hz, 1 H) 7.73 (d, J = 6.36 Hz, 1 H) 8.64 − 8.70 (m, 2 H) 9.45 (s, 1 H). MS (ES): m/z = 329.1 (M + H) |
| 33 | 2-((4′-bromo-[1,1′-biphenyl]-2-yl)oxy)-1,6-naphthyridine | ¹H NMR (400 MHz, DMSO-d₆) δ ppm |
| PP- | | 7.35 − 7.39 (m, 3 H) 7.40 − 7.43 (m, 1 H) |
| 1195 | 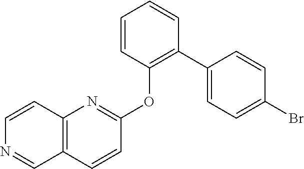 [SMILES: Brc1ccc(-c2ccccc2Oc2ccc3cnccc3n2)cc1] | 7.43 − 7.47 (m, 1 H) 7.47 − 7.50 (m, 2 H) 7.51 − 7.56 (m, 2 H) 7.66 (d, J = 6.11 Hz, 1 H) 8.60 (d, J = 9.54 Hz, 1 H) 8.63 (d, J = 6.11 Hz, 1 H) 9.38 (s, 1 H). MS (ES): m/z = 377.0 (M + H) |
| 34 | 2-([1,1′-biphenyl]-2-yloxy)-4-(trifluoromethyl)quinoline | ¹H NMR (400 MHz, DMSO-d₆) δ ppm |
| PP- | | 7.17 − 7.24 (m, 1 H) 7.25 − 7.31 (m, 2 H) |
| 1193 | 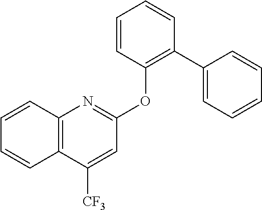 [SMILES: FC(F)(F)c1cc(Oc2ccccc2-c2ccccc2)nc2ccccc12] | 7.35 − 7.45 (m, 4 H) 7.51 (ddd, J = 16.20, 7.64, 1.83 Hz, 2 H) 7.60 − 7.66 (m, 2 H) 7.71 − 7.80 (m, 2 H) 7.96 (d, J = 8.31 Hz, 1 H). MS (ES): m/z = 366.1 (M + H) |
| 35 | 2-([1,1′-biphenyl]-2-yloxy)-8-methylquinoline | ¹H NMR (400 MHz, DMSO-d₆) δ ppm |
| PP- | | 2.31 (s, 3 H) 7.11 (d, J = 8.80 Hz, 1 H) |
| 1192 | 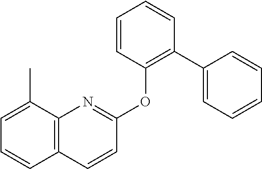 [SMILES: Cc1cccc2ccc(Oc3ccccc3-c3ccccc3)nc12] | 7.17 − 7.24 (m, 1 H) 7.26 − 7.35 (m, 4 H) 7.36 − 7.41 (m, 1 H) 7.42 − 7.49 (m, 4 H) 7.51 (dd, J = 7.58, 1.71 Hz, 1 H) 7.69 (d, J = 7.82 Hz, 1 H) 8.26 (d, J = 8.80 Hz, 1 H). MS (ES): m/z = 312.1 (M + H) |
| 36 | 7-methoxy-2-(2-(pyridin-2-yl)phenoxy)-4- | ¹H NMR (400 MHz, DMSO-d₆) δ ppm |
| PP- | (trifluoromethyl)quinoline | 3.85 (s, 3 H) 7.08 (d, J = 2.69 Hz, 1 H) |
| 1174 | | 7.21 − 7.29 (m, 2 H) 7.39 (dd, J = 8.07, |
| | 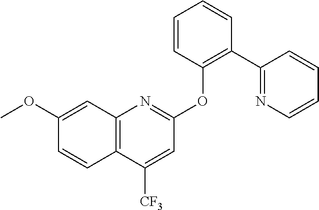 [SMILES: COc1ccc2c(C(F)(F)F)cc(Oc3ccccc3-c3ccccn3)nc2c1] | 0.98 Hz, 1 H) 7.45 (td, J = 7.58, 1.22 Hz, 1 H) 7.51 (s, 1 H) 7.53 − 7.59 (m, 1 H) 7.66 − 7.75 (m, 2 H) 7.84 − 7.89 (m, 2 H) 8.52 − 8.56 (m, 1 H). MS (ES): m/z = 397.1 (M + H) |
| 37 | N-([1,1′-biphenyl]-2-yl)-N,N-dimethyl-1,6-naphthyridin- | ¹H NMR (400 MHz, CHLOROFORM-d₆) |
| PP- | 2-aminium | δ ppm 4.22 (s, 3 H) 6.78 (d, J = 9.54 Hz, 1 |
| 1103 | | H) 7.18 − 7.35 (m, 5 H) 7.52 − 7.68 (m, 4 |
| | 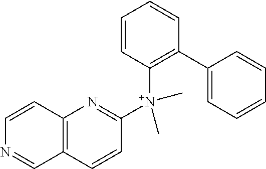 [SMILES: C[N+](C)(c1ccc2cnccc2n1)c1ccccc1-c1ccccc1] | H) 7.82 (d, J = 7.09 Hz, 1 H) 8.06 (d, J = 9.29 Hz, 1 H) 8.52 (dd, J = 7.09, 1.71 Hz, 1 H) 9.30 (d, J = 1.22 Hz, 1 H). MS (ES): m/z = 326.3 (M⁺) |
| 38 | N-([1,1′-biphenyl]-2-yl)-1,7-naphthyridin-2-amine | ¹H NMR (400 MHz, DMSO-d₆) δ ppm |
| PP- | | 7.07 (d, J = 9.05 Hz, 1 H) 7.20 − 7.26 (m, 1 |
| 1100 | 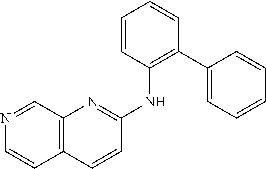 [SMILES: c1ccc(-c2ccccc2Nc2ccc3ccncc3n2)cc1] | H) 7.27 − 7.35 (m, 3 H) 7.36 − 7.45 (m, 4 H) 7.56 (dd, J = 5.38, 0.73 Hz, 1 H) 7.74 (dd, J = 7.95, 1.10 Hz, 1 H) 7.96 (dd, J = 8.93, 0.61 Hz, 1 H) 8.24 (d, J = 5.38 Hz, 1 H) 8.78 (s, 1 H) 8.88 (s, 1 H). MS (ES): m/z = 298.2 (M + H) |
| 39 | N-(4-fluorophenethyl)-7-methoxy-4- | ¹H NMR (400 MHz, DMSO-d₆) δ ppm |
| PP- | (trifluoromethyl)quinolin-2-amine | 2.91 (t, J = 7.21 Hz, 2 H) 3.59 − 3.67 (m, 2 |
| 1097 | | H) 3.87 (s, 3 H) 6.94 (dd, J = 9.05, 2.69 |
| | 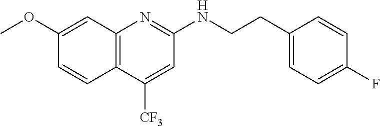 [SMILES: COc1ccc2c(C(F)(F)F)cc(NCCc3ccc(F)cc3)nc2c1] | Hz, 1 H) 7.03 (s, 1 H) 7.07 (d, J = 2.45 Hz, 1 H) 7.12 (t, J = 8.93 Hz, 2 H) 7.33 (dd, J = 8.80, 5.62 Hz, 2 H) 7.49 (br. s., 1 H) 7.62 (dd, J = 9.05, 2.20 Hz, 1 H). MS (ES): m/z = 365.2 (M + H) |
| 40 | 2-(4-(benzyloxy)phenoxy)-7-methoxy-4- | ¹H NMR (400 MHz, DMSO-d₆) δ ppm |
| PP- | (trifluoromethyl)quinoline | 3.87 (s, 3 H) 5.14 (s, 2 H) 7.09 − 7.11 (m, |
| 1096 | | 1 H) 7.11 − 7.14 (m, 2 H) 7.20 − 7.24 (m, |
| | 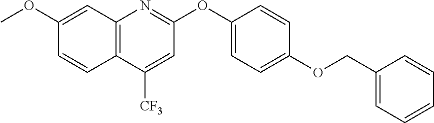 [SMILES: COc1ccc2c(C(F)(F)F)cc(Oc3ccc(OCc4ccccc4)cc3)nc2c1] | 2 H) 7.28 (dd, J = 9.29, 2.69 Hz, 1 H) 7.33 − 7.38 (m, 1 H) 7.39 − 7.46 (m, 2 H) 7.47 − 7.52 (m, 3 H) 7.90 (dd, J = 9.29, 2.20 Hz, 1 H). MS (ES): m/z = 426.1 (M + H) |
| 41 | 2-(2-cyclohexylphenoxy)-7-methoxy-4- | ¹H NMR (400 MHz, DMSO-d₆) δ ppm |
| PP- | (trifluoromethyl)quinoline | 1.12 − 1.21 (m, 3 H) 1.42 (d, J = 11.98 Hz, |
| 1095 | 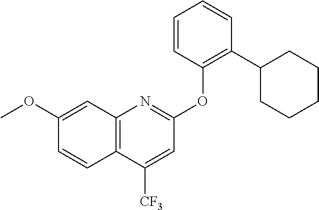 [SMILES: COc1ccc2c(C(F)(F)F)cc(Oc3ccccc3C3CCCCC3)nc2c1] | 2 H) 1.60 (br. s., 1 H) 1.69 (d, J = 8.56 Hz, 4 H) 2.64 − 2.74 (m, 1 H) 7.09 (d, J = 2.69 Hz, 1 H) 7.15 − 7.20 (m, 1 H) 7.24 − 7.31 (m, 3 H) 7.40 − 7.44 (m, 1 H) 7.57 (s, 1 H) 7.91 (dd, J = 9.17, 2.08 Hz, 1 H). MS (ES): m/z = 402.2 (M + H) |
| 42 | 2-([1,1′-biphenyl]-2-yloxy)-7-methoxy-4- | ¹H NMR (400 MHz, DMSO-d₆) δ ppm |
| PP- | (trifluoromethyl)quinoline | 3.86 (s, 3 H) 7.11 (d, J = 2.69 Hz, 1 H) |
| 1094 | | 7.20 − 7.34 (m, 4 H) 7.37 (dd, J = 8.07, |
| | 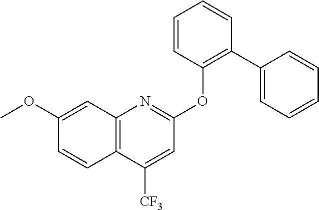 [SMILES: COc1ccc2c(C(F)(F)F)cc(Oc3ccccc3-c3ccccc3)nc2c1] | 0.98 Hz, 1 H) 7.38 − 7.45 (m, 4 H) 7.47 − 7.55 (m, 2 H) 7.85 (dd, J = 9.29, 1.96 Hz, 1 H). MS (ES): m/z = 396.1 (M + H) |
| 43 | 2-(2-(pyridin-2-yl)phenoxy)-1,7-naphthyridine | ¹H NMR (400 MHz, DMSO-d₆) δ ppm |
| PP- | | 7.21 (ddd, J = 7.09, 4.89, 1.47 Hz, 1 H) 7.39 |
| 1083 | 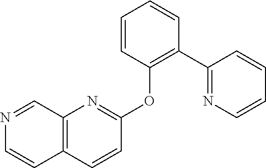 [SMILES: c1ccc(-c2ccccc2Oc2ccc3ccncc3n2)nc1] | (dd, J = 8.07, 1.22 Hz, 1 H) 7.42 − 7.51 (m, 2 H) 7.53 − 7.59 (m, 1 H) 7.62 − 7.72 (m, 2 H) 7.81 − 7.88 (m, 2 H) 8.42 (dd, J = 8.80, 0.49 Hz, 1 H) 8.46 − 8.56 (m, 2 H) 8.91 (s, 1 H). MS (ES): m/z = 300.1 (M + H) |
| 44 | 2-(2-(trifluoromethyl)phenoxy)-1,7-naphthyridine | ¹H NMR (400 MHz, DMSO-d₆) δ ppm |
| PP- | | 7.50 − 7.56 (m, 1 H) 7.58 (d, J = 8.31 Hz, 1 |
| 1082 | 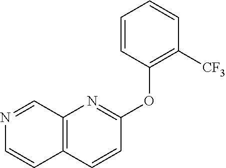 [SMILES: FC(F)(F)c1ccccc1Oc1ccc2ccncc2n1] | H) 7.64 (d, J = 8.80 Hz, 1 H) 7.82 (td, J = 7.95, 0.98 Hz, 1 H) 7.85 − 7.89 (m, 1 H) 7.92 (dd, J = 5.50, 0.86 Hz, 1 H) 8.51 − 8.57 (m, 2 H) 8.97 (s, 1 H). MS (ES): m/z = 291.1 (M + H) |
| 45 | 2-(2-(pyridin-2-yl)phenoxy)-1,6-naphthyridine | ¹H NMR (400 MHz, DMSO-d₆) δ ppm |
| PP- | | 7.18 − 7.24 (m, 1 H) 7.21 (ddd, J = 7.34, |
| 1081 | 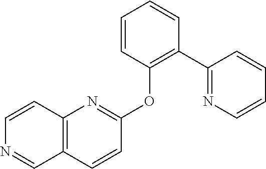 [SMILES: c1ccc(-c2ccccc2Oc2ccc3cnccc3n2)nc1] | 4.89, 1.22 Hz, 1 H) 7.34 − 7.40 (m, 2 H) 7.42 − 7.49 (m, 2 H) 7.53 − 7.59 (m, 1 H) 7.60 − 7.64 (m, 1 H) 7.66 − 7.72 (m, 1 H) 7.84 (dd, J = 7.70, 1.59 Hz, 1 H) 8.47 − 8.59 (m, 3 H) 9.22 (d, J = 0.73 Hz, 1 H): m/z = 300.1 (M + H) |
| 46 | 2-(2-(trifluoromethyl)phenoxy)-1,6-naphthyridine | ¹H NMR (400 MHz, DMSO-d₆) δ ppm |
| PP- | | 7.46 − 7.63 (m, 4 H) 7.75 − 7.93 (m, 2 H) |
| 1080 | 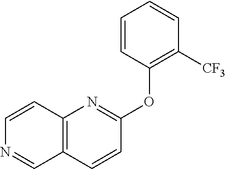 [SMILES: FC(F)(F)c1ccccc1Oc1ccc2cnccc2n1] | 8.56 − 8.71 (m, 2 H) 9.31 (d, J = 0.73 Hz, 1 H). MS (ES): m/z = 291.1 (M + H) |
| 47 | 2-(2-isopropylphenoxy)-1,7-naphthyridine | ¹H NMR (400 MHz, DMSO-d₆) δ ppm |
| PP- | | 1.13 (d, J = 6.85 Hz, 6 H) 3.02 (dt, |
| 1056 | 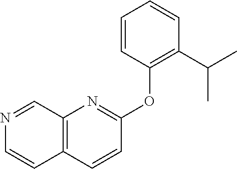 [SMILES: CC(C)c1ccccc1Oc1ccc2ccncc2n1] | J = 13.82, 7.03 Hz, 1 H) 7.16 − 7.21 (m, 1 H) 7.28 − 7.32 (m, 2 H) 7.44 − 7.49 (m, 1 H) 7.66 (d, J = 8.80 Hz, 1 H) 8.03 (dd, J = 5.62, 0.73 Hz, 1 H) 8.54 − 8.56 (m, 1 H) 8.57 (s, 1 H) 9.08 (s, 1 H). MS (ES): m/z = 265.1 (M + H) |
| 48 | 2-(2-cyclohexylphenoxy)-1,7-naphthyridine | ¹H NMR (400 MHz, DMSO-d₆) δ ppm |
| PP- | | 1.08 − 1.18 (m, 3 H) 1.35 − 1.47 (m, 3 H) |
| 1055 | 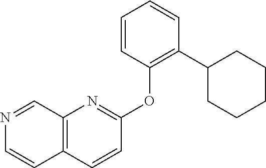 [SMILES: c1ccc(C2CCCCC2)c(Oc2ccc3ccncc3n2)c1] | 1.38 − 1.47 (m, 2 H) 1.60 (d, J = 9.78 Hz, 1 H) 1.68 (d, J = 6.36 Hz, 5 H) 2.64 (t, J = 11.98 Hz, 1 H) 7.17 − 7.20 (m, 1 H) 7.27 − 7.30 (m, 2 H) 7.42 − 7.45 (m, 1 H) 7.65 (d, J = 8.80 Hz, 1 H) 8.01 (dd, J = 5.62, 0.73 Hz, 1 H) 8.53 − 8.55 (m, 1 H) 8.56 (s, 1 H) 9.06 (s, 1 H). MS (ES): m/z = 305.2 (M + H) |
| 49 | 2-(2-cyclohexylphenoxy)-1,6-naphthyridine | ¹H NMR (400 MHz, DMSO-d₆) δ ppm |
| PP- | | 1.06 − 1.18 (m, 3 H) 1.35 − 1.47 (m, 2 H) |
| 1054 | 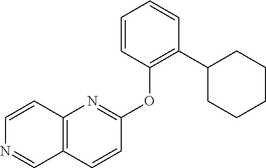 [SMILES: c1ccc(C2CCCCC2)c(Oc2ccc3cnccc3n2)c1] | 1.55 − 1.63 (m, 1 H) 1.67 (d, J = 10.51 Hz, 4 H) 2.60 (t, J = 12.10 Hz, 1 H) 7.20 (s, 1 H) 7.26 − 7.33 (m, 2 H) 7.43 (s, 1 H) 7.57 (d, J = 8.80 Hz, 1 H) 7.72 (d, J = 6.36 Hz, 1 H) 8.66 (d, J = 6.36 Hz, 1 H) 8.69 − 8.73 (m, 1 H) 9.46 (s, 1 H). MS (ES): m/z = 305.2 (M + H) |
| 50 | 2-([1,1′-biphenyl]-4-yloxy)-1,7-naphthyridine | ¹H NMR (400 MHz, DMSO-d₆) δ ppm |
| PP- | | 7.36 − 7.42 (m, 3 H) 7.47 − 7.52 (m, 2 H) |
| 1047 | 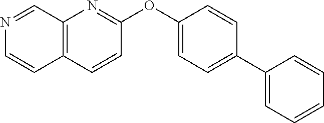 [SMILES: c1ccc(-c2ccc(Oc3ccc4ccncc4n3)cc2)cc1] | 7.59 (d, J = 9.05 Hz, 1 H) 7.70 − 7.75 (m, 2 H) 7.76 − 7.81 (m, 2 H) 7.91 (dd, J = 5.50, 0.86 Hz, 1 H) 8.48 − 8.57 (m, 2 H) 9.01 (s, 1 H). MS (ES): m/z = 299.2 (M + H) |
| 51 | 2-([1,1′-biphenyl]-3-yloxy)-1,7-naphthyridine | ¹H NMR (400 MHz, DMSO-d₆) δ ppm |
| PP- | | 7.31 (ddd, J = 7.70, 2.32, 1.47 Hz, 1 H) |
| 1046 | 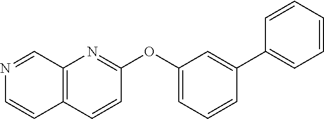 [SMILES: c1ccc(-c2cccc(Oc3ccc4ccncc4n3)c2)cc1] | 7.35 − 7.42 (m, 1 H) 7.44 − 7.51 (m, 2 H) 7.55 − 7.66 (m, 4 H) 7.69 − 7.75 (m, 2 H) 7.98 (dd, J = 5.62, 0.73 Hz, 1 H) 8.52 − 8.58 (m, 2 H) 9.07 (s, 1 H). MS (ES): m/z = 299.2 (M + H) |
| 52 | 2-([1,1′-biphenyl]-4-yloxy)-1,6-naphthyridine | ¹H NMR (400 MHz, DMSO-d₆) δ ppm |
| PP- | | 7.36 − 7.44 (m, 3 H) 7.47 − 7.53 (m, 2 H) |
| 1045 | 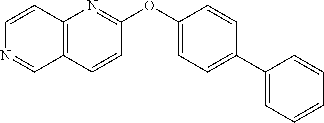 [SMILES: c1ccc(-c2ccc(Oc3ccc4cnccc4n3)cc2)cc1] | 7.58 (d, J = 9.05 Hz, 1 H) 7.71 − 7.75 (m, 2 H) 7.75 − 7.82 (m, 3 H) 8.66 − 8.76 (m, 2 H) 9.48 (s, 1 H). MS (ES): m/z = 299.2 (M + H) |
| 53 | 2-([1,1′-biphenyl]-3-yloxy)-1,6-naphthyridine | ¹H NMR (400 MHz, DMSO-d₆) δ ppm |
| PP- | | 7.32 (ddd, J = 7.89, 2.26, 1.10 Hz, 1 H) |
| 1044 | 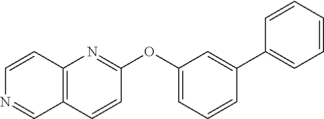 [SMILES: c1ccc(-c2cccc(Oc3ccc4cnccc4n3)c2)cc1] | 7.36 − 7.42 (m, 1 H) 7.44 − 7.51 (m, 2 H) 7.57 − 7.68 (m, 4 H) 7.70 − 7.75 (m, 2 H) 7.82 (d, J = 6.11 Hz, 1 H) 8.70 (d, J = 6.11 Hz, 1 H) 8.75 (dd, J = 9.05, 0.73 Hz, 1 H) 9.52 (s, 1 H). MS (ES): m/z = 299.2 (M + H) |
| 54 | 2-(naphthalen-1-yloxy)-1,7-naphthyridine | ¹H NMR (400 MHz, DMSO-d₆) δ ppm |
| PP- | | 7.47 − 7.53 (m, 2 H) 7.55 − 7.66 (m, 2 H) |
| 1029 | 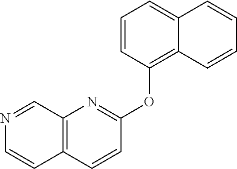 [SMILES: c1ccc2c(Oc3ccc4ccncc4n3)cccc2c1] | 7.82 (d, J = 8.80 Hz, 1 H) 7.87 (d, J = 8.31 Hz, 1 H) 7.93 (d, J = 8.31 Hz, 1 H) 8.02 − 8.08 (m, 2 H) 8.56 (s, 1 H) 8.60 − 8.66 (m, 1 H) 8.97 (s, 1 H). MS (ES): m/z = 273.2 (M + H) |
| 55 | 2-(naphthalen-2-yloxy)-1,7-naphthyridine | ¹H NMR (400 MHz, DMSO-d₆) δ ppm |
| PP- | | 7.48 (dd, J = 8.80, 2.45 Hz, 1 H) 7.52 − |
| 1028 | 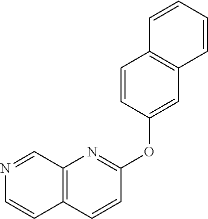 [SMILES: c1ccc2cc(Oc3ccc4ccncc4n3)ccc2c1] | 7.60 (m, 2 H) 7.62 (d, J = 8.80 Hz, 1 H) 7.71 (d, J = 6.11 Hz, 1 H) 7.85 (d, J = 2.45 Hz, 1 H) 7.91 − 8.03 (m, 2 H) 8.07 (s, 1 H) 8.67 (d, J = 6.11 Hz, 1 H) 8.74 (dd, J = 8.80, 0.73 Hz, 1 H) 9.49 (s, 1 H). MS (ES): m/z = 273.1 (M + H) |
| 56 | 2-(naphthalen-1-yloxy)-1,6-naphthyridine | ¹H NMR (400 MHz, DMSO-d₆) δ ppm |
| PP- | | 7.48 − 7.54 (m, 2 H) 7.59 (ddd, J = 8.25, |
| 1027 | 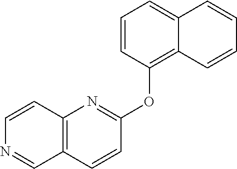 [SMILES: c1ccc2c(Oc3ccc4cnccc4n3)cccc2c1] | 6.91, 1.22 Hz, 1 H) 7.62 − 7.69 (m, 2 H) 7.76 (d, J = 9.05 Hz, 1 H) 7.81 − 7.86 (m, 1 H) 7.95 (d, J = 8.31 Hz, 1 H) 8.06 (d, J = 8.07 Hz, 1 H) 8.65 (d, J = 6.36 Hz, 1 H) 8.80 (dd, J = 9.05, 0.73 Hz, 1 H) 9.53 (s, 2 H). MS (ES): m/z = 273.1 (M + H) |
| 57 | 2-(cyclohexyloxy)-1,7-naphthyridine | ¹H NMR (400 MHz, DMSO-d₆) δ ppm |
| PP- | | 1.24 − 1.63 (m, 6 H) 1.77 (dd, J = 8.68, |
| 1003 | 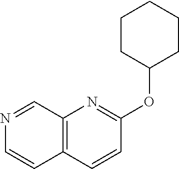 [SMILES: c1cc2ccc(OC3CCCCC3)nc2cn1] | 4.52 Hz, 2 H) 2.05 (dd, J = 12.23, 4.16 Hz, 2 H) 5.19 − 5.37 (m, 1 H) 7.30 (d, J = 9.05 Hz, 1 H) 7.94 (d, J = 5.62 Hz, 1 H) 8.35 (dd, J = 9.05, 0.49 Hz, 1 H) 8.51 (d, J = 5.62 Hz, 1 H) 9.19 (s, 1 H). MS (ES): m/z = 229.2 (M + H) |
| 58 | 2-cyclobutoxy-1,7-naphthyridine | ¹H NMR (400 MHz, DMSO-d₆) δ ppm |
| PP- | | 1.61 − 1.90 (m, 2 H) 2.08 − 2.24 (m, 2 H) |
| 1002 | 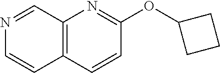 [SMILES: c1cc2ccc(OC3CCC3)nc2cn1] | 5.36 (dd, J = 7.83, 7.09 Hz, 2 H) 7.37 (s, 1 H) 8.00 (d, J = 5.62 Hz, 1 H) 8.39 (dd, J = 9.05, 0.49 Hz, 1 H) 8.55 (d, J = 5.62 Hz, 1 H) 9.23 (s, 1 H). MS (ES): m/z = 201.2 (M + H) |
| 59 | 2-([1,1′-biphenyl]-2-yloxy)-1,7-naphthyridine | ¹H NMR (400 MHz, DMSO-d₆) δ ppm |
| PP- | | 7.18 − 7.23 (m, 1 H) 7.25 − 7.31 (m, 2 H) |
| 1001 | 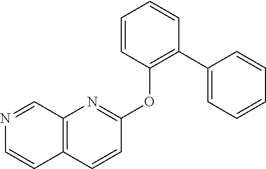 [SMILES: c1ccc(-c2ccccc2Oc2ccc3ccncc3n2)cc1] | 7.35 − 7.45 (m, 4 H) 7.46 − 7.56 (m, 3 H) 7.94 (d, J = 4.89 Hz, 1 H) 8.43 (dd, J = 8.93, 0.61 Hz, 1 H) 8.52 (d, J = 5.38 Hz, 1 H) 9.05 (s, 1 H). MS (ES): m/z = 299.2 (M + H) |
| 60 | 2-((7-methoxy-4-(trifluoromethyl)quinolin-2-yl)thio)-N- | ¹H NMR (400 MHz, CHLOROFORM-d) |
| PP-979 | (2-morpholinoethyl)acetamide | δ ppm 3.19 (t, J = 5.75 Hz, 2 H) 3.69 (q, |
| | | J = 6.03 Hz, 2 H) 3.81 − 3.86 (m, 4 H) 4.01 |
| | 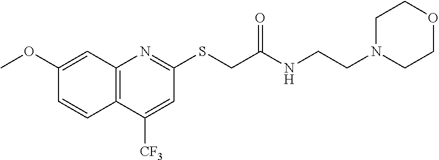 [SMILES: COc1ccc2c(C(F)(F)F)cc(SCC(=O)NCCN3CCOCC3)nc2c1] | (s, 3 H) 4.04 (s, 2 H) 7.23 (dd, J = 9.29, 2.45 Hz, 1 H) 7.42 (s, 1 H) 7.51 (d, J = 2.69 Hz, 1 H) 7.93 (dd, J = 9.29, 1.96 Hz, 1 H) 8.10 (t, J = 5.99 Hz, 1 H). MS (ES): m/z = 430.1 (M + H) |
| 61 | 2-((7-methoxy-4-(trifluoromethyl)quinolin-2-yl)thio)-1- | ¹H NMR (400 MHz, DMSO-d₆) δ ppm |
| PP-978 | morpholinoethan-1-one | 3.48 (d, J = 5.14 Hz, 2 H) 3.58 (d, J = 5.14 |
| | | Hz, 2 H) 3.68 (s, 4 H) 3.95 (s, 3 H) 4.40 |
| | 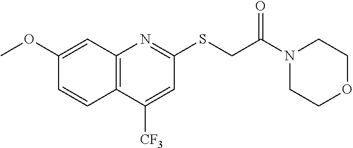 [SMILES: COc1ccc2c(C(F)(F)F)cc(SCC(=O)N3CCOCC3)nc2c1] | (s, 2 H) 7.34 (dd, J = 9.29, 2.69 Hz, 1 H) 7.39 (d, J = 2.45 Hz, 1 H) 7.73 (s, 1 H) 7.89 (dd, J = 9.29, 2.20 Hz, 1 H). MS (ES): m/z = 387.1 (M + H) |
| 62 | N-(4-fluorophenethyl)-2-((7-methoxy-4- | ¹H NMR (400 MHz, DMSO-d₆) δ ppm |
| PP-977 | (trifluoromethyl)quinolin-2-yl)thio)acetamide | 2.69 (t, J = 7.09 Hz, 2 H) 3.27 − 3.32 (m, 2 |
| | | H) 3.93 (s, 3 H) 4.01 (s, 2 H) 6.92 (t, |
| | 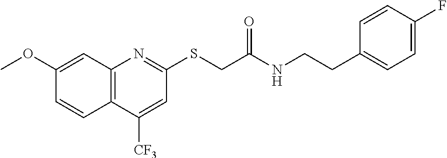 [SMILES: COc1ccc2c(C(F)(F)F)cc(SCC(=O)NCCc3ccc(F)cc3)nc2c1] | J = 8.93 Hz, 2 H) 7.14 (dd, J = 8.80, 5.62 Hz, 2 H) 7.34 (dd, J = 9.29, 2.69 Hz, 1 H) 7.40 (d, J = 2.69 Hz, 1 H) 7.70 (s, 1 H) 7.89 (dd, J = 9.29, 1.96 Hz, 1 H) 8.25 (t, J = 5.62 Hz, 1H). MS (ES): m/z = 439.1 (M + H) |
| 63 | 2-((7-methoxy-4-(trifluoromethyl)quinolin-2-yl)thio)-1- | ¹H NMR (400 MHz, DMSO-d₆) δ ppm |
| PP-976 | (3-(3-(thiophen-2-yl)-1,2,4-oxadiazol-5-yl)azetidin-1- | 3.90 (s, 3 H) 4.10 (s, 1 H) 4.13 (s, 1 H) |
| | yl)ethan-1-one | 4.16 − 4.19 (m, 1 H) 4.37 (d, J = 5.14 Hz, 2 |
| | | H) 4.73 (dd, J = 8.80, 4.89Hz, 1 H) 4.88 (t, |
| | 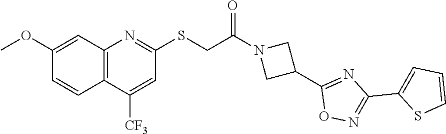 [SMILES: COc1ccc2c(C(F)(F)F)cc(SCC(=O)N3CC(c4nc(-c5cccs5)no4)C3)nc2c1] | J = 8.31 Hz, 1 H) 7.27 (dd, J = 5.14, 3.67 Hz, 1 H) 7.34 (dd, J = 9.29, 2.69 Hz, 1 H) 7.39 (d, J = 2.69 Hz, 1 H) 7.74 (s, 1 H) 7.78 (dd, J = 3.67, 1.22 Hz, 1 H) 7.87 − 7.91 (m, 2 H). MS (ES): m/z = 507.1 (M + H) |
| 64 | 2-(cyclohexyloxy)-1,6-naphthyridine | ¹H NMR (400 MHz, DMSO-d₆) δ ppm |
| PP-972 | | 1.26 − 1.38 (m, 1 H) 1.38 − 1.50 (m, 2 H) |
| | 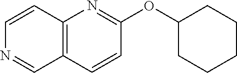 [SMILES: c1cc2nc(OC3CCCCC3)ccc2cn1] | 1.51 − 1.63 (m, 3 H) 1.70 − 1.83 (m, 2 H) 1.96 − 2.10 (m, 2 H) 5.27 − 5.41 (m, 1 H) 7.22 (d, J = 9.05 Hz, 1 H) 7.84 (d, J = 6.11 Hz, 1 H) 8.50 (dd, J = 8.93, 0.61 Hz, 1 H) 8.68 (d, J = 6.11 Hz, 1 H) 9.37 (s, 1 H). |
| | | MS (ES): m/z = 229.2 (M + H) |
| 65 | 2-cyclobutoxy-1,6-naphthyridine | ¹H NMR (400 MHz, CHLOROFORM-d₆) |
| PP-971 | | δ ppm 1.70 − 1.87 (m, 1 H) 1.88 − 2.03 |
| | 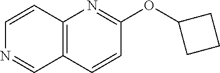 [SMILES: c1cc2nc(OC3CCC3)ccc2cn1] | (m, 1 H) 2.15 − 2.37 (m, 2 H) 2.49 − 2.68 (m, 2 H) 5.40 − 5.59 (m, 1 H) 7.19 (d, J = 9.05 Hz, 1 H) 7.97 (d, J = 6.60 Hz, 1 H) 8.27 (dd, J = 9.05, 0.73 Hz, 1 H) 8.65 (d, J = 6.36 Hz, 1 H) 9.46 (s, 1 H). MS (ES): |
| | | m/z = 201.2 (M + H) |
| 66 | 2-([1,1′-biphenyl]-2-yloxy)-1,6-naphthyridine | ¹H NMR (400 MHz, DMSO-d₆) δ ppm |
| PP-968 | | 7.18 − 7.24 (m, 1 H) 7.25 − 7.30 (m, 3 H) |
| | 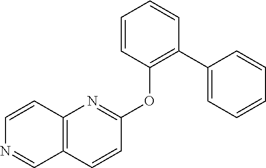 [SMILES: c1ccc(-c2ccccc2Oc2ccc3cnccc3n2)cc1] | 7.35 (dd, J = 7.95, 1.35 Hz, 1 H) 7.38 − 7.44 (m, 3 H) 7.47 − 7.54 (m, 3 H) 8.48 (dd, J = 8.93, 0.61 Hz, 1 H) 8.56 (d, J = 5.87 Hz, 1 H) 9.20 (s, 1 H). MS (ES): m/z = 299.1 (M + H) |
| 67 | 6-([1,1′-biphenyl]-2-yloxy)-1,7-naphthyridine | MS (ES): m/z = 299.1 (M + H) |
| PP-959 | | |
| | 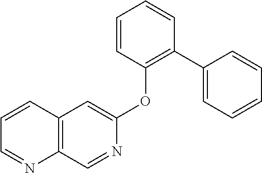 [SMILES: c1ccc(-c2ccccc2Oc2cc3cccnc3cn2)cc1] | |
| 68 | 2-([1,1′-biphenyl]-2-yloxy)-1,5-naphthyridine | ¹H NMR (400 MHz, DMSO-d₆) δ ppm |
| PP-958 | | 7.18 − 7.31 (m, 3 H) 7.33 − 7.44 (m, 4 H) |
| | 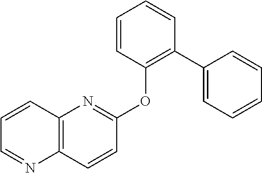 [SMILES: c1ccc(-c2ccccc2Oc2ccc3ncccc3n2)cc1] | 7.46 − 7.55 (m, 2 H) 7.64 (dd, J = 8.44, 4.28 Hz, 1 H) 7.97 − 8.08 (m, 1 H) 8.28 − 8.39 (m, 1 H) 8.81 (dd, J = 4.28, 1.59 Hz, 1 H). MS (ES): m/z = 299.1 (M + H) |
| 69 | N-(4-(5-methyl-1H-pyrazol-1-yl)phenyl)-1,7- | ¹H NMR (400 MHz, DMSO-d₆) δ ppm |
| PP- | naphthyridin-2-amine | 2.31 (s, 3 H) 6.37 (s, 1 H) 7.31 (d, J = 8.80 |
| 1281 | | Hz, 1 H) 7.37 − 7.49 (m, 2 H) 7.70 (d, |
| | 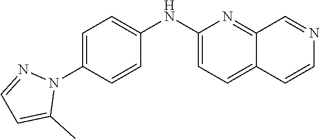 [SMILES: Cc1ccnn1-c1ccc(Nc2ccc3ccncc3n2)cc1] | J = 5.14 Hz, 1 H) 8.02 (d, J = 8.07 Hz, 1 H) 8.16 (d, J = 9.05 Hz, 1 H) 8.32 − 8.41 (m, 2 H) 8.46 (s, 1 H) 9.07 (s, 1 H) 9.93 (s, 1 H). MS (ES): m/z = 302.1 (M + H) |
| 70 | 2-(naphthalen-2-yloxy)-1,7-naphthyridine | ¹H NMR (400 MHz, DMSO-d₆) δ ppm |
| PP- | | 7.50 (dd, J = 8.93, 2.32 Hz, 1 H) 7.52 − |
| 1030 | 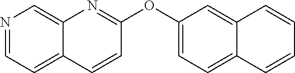 [SMILES: c1ccc2cc(Oc3ccc4ccncc4n3)ccc2c1] | 7.60 (m, 2 H) 7.71 (d, J = 9.05 Hz, 1 H) 7.85 (d, J = 2.45 Hz, 1 H) 7.93 − 7.97 (m, 1 H) 7.99 − 8.03 (m, 1 H) 8.03 − 8.08 (m, 2 H) 8.53 − 8.64 (m, 2 H) 9.07 (s, 1 H). MS (ES): m/z = 273.1 (M + H) |
| 71 | 2-((3,5-dimethyl-[1,1′-biphenyl]-2-yl)oxy)-1,6- | ¹H NMR (400 MHz, CD₃OD) δ ppm |
| | naphthyridine | 9.31 (s, 1H), 8.58 (d, J = 6.38 Hz, |
| | | 1H), 8.49 (d, J = 8.98 1H), 7.80 (d, J = |
| | 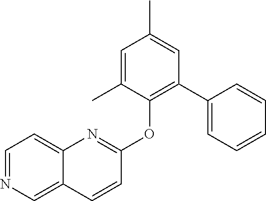 [SMILES: Cc1cc(C)c(Oc2ccc3cnccc3n2)c(-c2ccccc2)c1] | 6.38 Hz, 1H), 7.37 (d, J = 7.04 Hz, 2H), 7.33 (d, J = 8.97 Hz,1H), 7.18 − 7.22 (m, 3H), 7.13 − 7.15 (m, 2H), 2.44 (s, 3H), 2.19 (s, 3H). MS (ES): m/z = 327.1 [M + H⁺] |
| 72 | 6-((1,6-naphthyridin-2-yl)oxy)-N,N-diethyl-4′-methoxy- | ¹H NMR (400 MHz, CD₃OD) δ ppm |
| | [1,1′-biphenyl]-3-amine | 9.28 (s, 1H), 8.68 (d, , J = 6.01 Hz, |
| | | 1H), 8.55 (d, J = 8.64 Hz, 1H), 8.22 |
| | 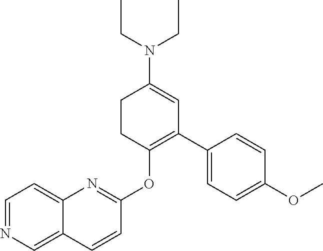 [SMILES: CCN(CC)C1=CC(c2ccc(OC)cc2)=C(Oc2ccc3cnccc3n2)CC1] | (d, J = 8.90 Hz, 2H), 8.19 (d, J = 8.74 Hz, 1H), 7.99 (d, J = 6.00 Hz, 1H), 7.14 (d, J = 8.90 Hz, 2H), 3.93 (s, 3H). MS (ES): m/z = 400.2 [M + H⁺] |
| 73 | 2-((3-chloro-5-methyl-[1,1′-biphenyl]-2-yl)oxy)-1,6- | ¹H NMR (400 MHz, (CD₃OD) δ ppm |
| | naphthyridine | 9.08 (s, 1H), 8.50 (d, J = 5.96 Hz, |
| | | 1H), 8.36 (d, J = 8.88 Hz, 1H), 7.54 |
| | 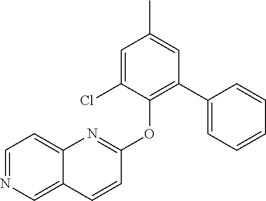 [SMILES: Cc1cc(Cl)c(Oc2ccc3cnccc3n2)c(-c2ccccc2)c1] | (d, J = 6.00 Hz, 1H), 7.37 (d, J = 7.32 Hz, 3H), 7.20 − 7.23 (m, 3H), 7.17 − 7.18 (m, 2H), 2.44 (s, 3H). MS (ES): m/z = 347.1 [M + H⁺] |
| 74 | 2-((3-chloro-[1,1′-biphenyl]-2-yl)oxy)-1,6-naphthyridine | ¹H NMR (400 MHz, (CD₃OD) δ ppm |
| | | 9.02 (s, 1H), 8.46 (d, J = 5.96 Hz, |
| | 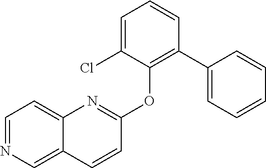 [SMILES: Clc1cccc(-c2ccccc2)c1Oc1ccc2cnccc2n1] | 1H), 8.29 (d, J = 8.92 Hz, 1H), 7.51 (d, J = 5.96 Hz, 1H), 7.35 (s, 1H), 7.33 (s, 2H), 7.29 (d, J = 7.16 Hz, 2H), 7.16 (t, J = 7.44 Hz, 2H), 7.09 (d, J = 5.52 Hz, 2H), 2.18 (s, 3H). MS (ES): m/z = 333.1 [M + H⁺] |
| 75 | 2′-((1,6-naphthyridin-2-yl)oxy)-3′-chloro-[1,1′-biphenyl]- | ¹H NMR (400 MHz, (CD₃OD) δ ppm |
| | 3-carbonitrile | 9.14 (s, 1H), 8.52 − 8.54 (m, 1H), |
| | | 8.44 (d, J = 8.88 Hz, 1H), 7.79 (s, |
| | 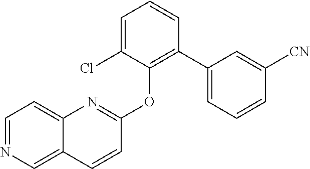 [SMILES: N#Cc1cccc(-c2cccc(Cl)c2Oc2ccc3cnccc3n2)c1] | 1H), 7.73 (d, J = 7.96 Hz, 1H), 7.64 (dd, J = 7.36 Hz, 2.20 Hz, 1H), 7.54 − 7.58 (m, 2H), 7.45 − 7.48 (m, 2H), 7.41 − 7.43 (m, 1H), 7.29 (d, J = 8.88 Hz, 1H MS (ES): m/z = 358.1 [M + H⁺] |
| 76 | 2-((3,3′-difluoro-[1,1′-biphenyl]-2-yl)oxy)-1,6- | ¹H NMR (400 MHz, (CD₃OD) δ ppm |
| | naphthyridine | 9.08 (s, 1H), 8.50 (d, J = 6.00 Hz, |
| | | 1H), 8.37 (d, J = 8.88 Hz, 1H), 6.44 |
| | 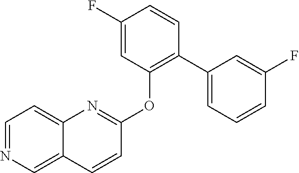 [SMILES: Fc1cccc(-c2ccc(F)cc2Oc2ccc3cnccc3n2)c1] | Hz, 2H), 7.16 − 7.24 (m, 4H), 7.10 − 7.16 (m, 2H), 6.88 (t, J = 7.88 Hz, 1H). MS (ES): m/z = 335.1 [M + H⁺] |
| 77 | 2-((5,3′-difluoro-[1,1′-biphenyl]-2-yl)oxy)-1,6- | ¹H NMR (400 MHz, (CD₃OD) δ ppm |
| | naphthyridine | 9.08 (s, 1H), 8.51 (d, J = 5.96 Hz, |
| | | 1H), 8.37 (d, J = 8.88 Hz, 1H), 7.53 |
| | 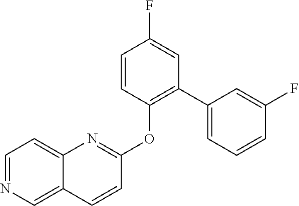 [SMILES: Fc1cccc(-c2cc(F)ccc2Oc2ccc3cnccc3n2)c1] | (d, J = 5.96 Hz, 1H), 7.32 − 7.35 (m, 1H), 7.26 −7.29 (m, 2H), 7.19 − 7.24 (m, 3H), 7.16 (d, J = 10.32 Hz, 1H), 6.92 (t, J = 6.84 Hz, 1H). MS (ES): m/z = 335.1 [M + H⁺] |
| 78 | 2-((4′-methoxy-4-(trifluoromethyl)-[1,1′-biphenyl]-2- | ¹H NMR (400 MHz, CD₃OD) δ ppm |
| | yl)oxy)-1,6-naphthyridine | 9.07 (s, 1H), 8.48 (d, J = 5.98 Hz, |
| | | 1H), 8.36 (d, J = 8.87 Hz, 1H), 7.64 − |
| | 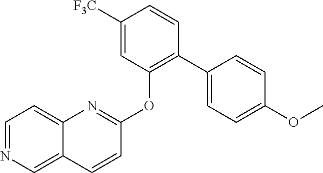 [SMILES: COc1ccc(-c2ccc(C(F)(F)F)cc2Oc2ccc3cnccc3n2)cc1] | 7.69 (m, 2H), 7.61 (s, 1H), 7.51 (d, J = 5.99 Hz, 1H), 7.36 (d, J = 8.85 Hz, 2H), 7.22 (d, J = 8.88 Hz, 2H), 6.76 (d, J = 8.85 Hz, 2H), 3.68 (s, 3H). MS (ES): m/z = 397.1 [M + H⁺] |
| 79 | 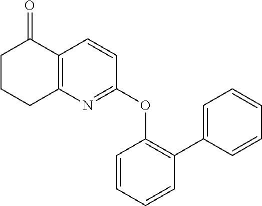 [SMILES: O=C1CCCc2nc(Oc3ccccc3-c3ccccc3)ccc21] | |
| 80 | 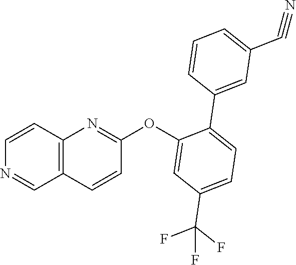 [SMILES: N#Cc1cccc(-c2ccc(C(F)(F)F)cc2Oc2ccc3cnccc3n2)c1] | |
| 81 | 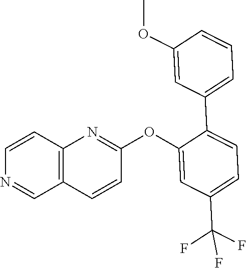 [SMILES: COc1cccc(-c2ccc(C(F)(F)F)cc2Oc2ccc3cnccc3n2)c1] | |
| 82 | 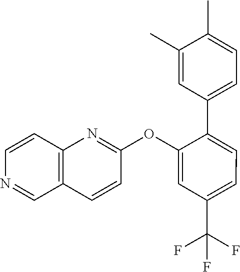 [SMILES: Cc1ccc(-c2ccc(C(F)(F)F)cc2Oc2ccc3cnccc3n2)cc1C] | |
| 83 | 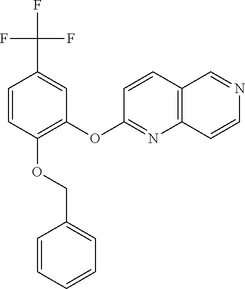 [SMILES: FC(F)(F)c1ccc(OCc2ccccc2)c(Oc2ccc3cnccc3n2)c1] | |
| 84 | 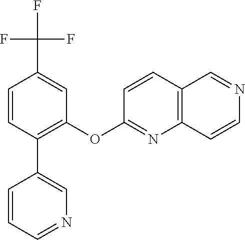 [SMILES: FC(F)(F)c1ccc(-c2cccnc2)c(Oc2ccc3cnccc3n2)c1] | |
| 85 | 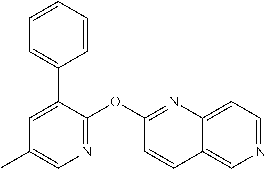 [SMILES: Cc1cnc(Oc2ccc3cnccc3n2)c(-c2ccccc2)c1] | |
| 86 | 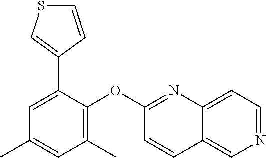 [SMILES: Cc1cc(C)c(Oc2ccc3cnccc3n2)c(-c2ccsc2)c1] | |
| 87 | 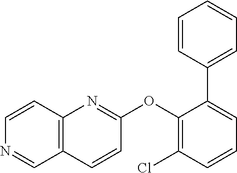 [SMILES: Clc1cccc(-c2ccccc2)c1Oc1ccc2cnccc2n1] | |
| 88 | 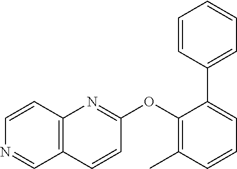 [SMILES: Cc1cccc(-c2ccccc2)c1Oc1ccc2cnccc2n1] | |
| 89 | 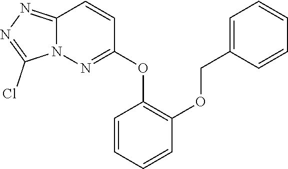 [SMILES: Clc1nnc2ccc(Oc3ccccc3OCc3ccccc3)nn12] | |
| 90 | 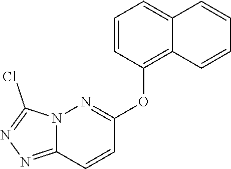 [SMILES: Clc1nnc2ccc(Oc3cccc4ccccc34)nn12] | |
| 91 | 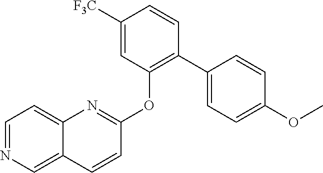 [SMILES: COc1ccc(-c2ccc(C(F)(F)F)cc2Oc2ccc3cnccc3n2)cc1] | |

### Biological Examples

### Example A: FRET Assay for REV-ERB Ligands

The capacity for compounds to function as ligands for either REV-ERBα or REV-ERBβ was assessed using a fluorescent resonance energy transfer assay that detects the interaction between these receptors and the Nuclear Receptor Corepressor (NCoR) protein (the ID2 corepressor interaction domain peptides is used). This interaction is known to be ligand dependent and thus this assay that can detect an alteration of the affinity of these two proteins is able to detect ligands. The His-tagged ligand binding domain (LBD) of either REV-ERBα or REV-ERBβ and fluorescein labeled NCoR ID2 peptide (Life Technologies #PV4624) is used in these assays. The His-tagged LBDs were expressed in E. coli (amino acids 281-614 REV-ERBα and 381-579 REV-ERBβ) and labeled with a Terbium (Tb) labeled anti-His antibody (Life Technologies #PV5895). The assay buffer was PBS (phosphate buffered saline) with 5 mM DTT (dithiothreitol). The final concentration of various reagents in the assay are: REV-ERB LBD (either isoform) [5 nM], Fluorescein labeled NCoR ID2 peptide [250 nM], Tb labeled anti-his antibody [10 nM], dimethylsulfoxide [1%] and test compound [varying concentrations]. The assay was performed in Corning NBS black 384-well plates in a total volume of 20 μl. The assay plate was incubated for 1 hour at room temperature protected from light and then TR-FRET was assessed on a Biotek plate reader with the following excitation/emission pairs (340 nm/495 nm and 340 nm/520 nm. The 520 nm/495 nm emission signal ratio was used as an indicator of the degree of interaction between the LBD and NCoR peptide and EC₅₀'s were calculated using GraphPad Prizm software.

The results of testing in the FRET Assay are summarized in Table 2. In Table 2, “++” indicates an IC₅₀≤0.25 μM, “+” indicates an IC₅₀≤1 μM, and “−” indicates an IC₅₀>1 μM.

TABLE 2

| Example | RevErbα Inhibition IC₅₀ μM | RevErbβ Inhibition IC₅₀ μM |
|---|---|---|
| 1 | | |
| CDD-1285 | | |
| 2 | | |
| CDD-1289 | | |
| 3 | + | ++ |
| CDD-1316 | | |
| 4 | | ++ |
| CDD-1459 | | |
| 5 | ++ | + |
| CDD-1496 | | |
| 6 | ++ | |
| CDD-1317 | | |
| 7 | | |
| CDD-1304 | | |
| 8 | | |
| CDD-1450 | | |
| 9 | | |
| CDD-1430 | | |
| 10 | ++ | ++ |
| CDD-1427 | | |
| 11 | | |
| CDD-1429 | | |
| 12 | | |
| CDD-1309 | | |
| 13 | − | |
| CDD-1450 | | |
| 14 | + | |
| CDD-1477 | | |
| 15 | | |
| CDD-1306 | | |
| 16 | | |
| CDD-1399 | | |
| 17 | ++ | |
| CDD-1426 | | |
| 18 | | |
| CDD-1460 | | |
| 19 | | |
| CDD-1479 | | |
| 20 | | |
| CDD-1478 | | |
| 21 | − | |
| CDD-1435 | | |
| 22 | ++ | |
| CDD-1449 | | |
| 23 | | |
| CDD-1506 | | |
| 24 | ++ | |
| CDD-1487 | | |
| 25 | | |
| CDD-1497 | | |
| 26 | | |
| SLUPP_1101 | | |
| 27 | | |
| SLUPP-1102 | | |
| 28 | | |
| SLUPP-975 | | |
| 29 | | |
| PP-1199 | | |
| 30 | | |
| PP-1198 | | |
| 31 | | |
| PP-1197 | | |
| 32 | | |
| PP-1196 | | |
| 33 | | |
| PP-1195 | | |
| 34 | | − |
| PP-1193 | | |
| 35 | | |
| PP-1192 | | |
| 36 | ++ | |
| PP-1174 | | |
| 37 | | |
| PP-1103 | | |
| 38 | | |
| PP-1100 | | |
| 39 | | |
| PP-1097 | | |
| 40 | | |
| PP-1096 | | |
| 41 | | |
| PP-1095 | | |
| 42 | | |
| PP-1094 | | |
| 43 | | |
| PP-1083 | | |
| 44 | | ++ |
| PP-1082 | | |
| 45 | − | ++ |
| PP-1081 | | |
| 46 | + | − |
| PP-1080 | | |
| 47 | | |
| PP-1056 | | |
| 48 | ++ | |
| PP-1055 | | |
| 49 | + | |
| PP-1054 | | |
| 50 | | |
| PP-1047 | | |
| 51 | | − |
| PP-1046 | | |
| 52 | | |
| PP-1045 | | |
| 53 | | |
| PP-1044 | | |
| 54 | ++ | |
| PP-1029 | | |
| 55 | | |
| PP-1028 | | |
| 56 | − | − |
| PP-1027 | | |
| 57 | − | |
| PP-1003 | | |
| 58 | | |
| PP-1002 | | |
| 59 | ++ | ++ |
| PP-1001 | | |
| 60 | | |
| PP-979 | | |
| 61 | | |
| PP-978 | | |
| 62 | ++ | |
| PP-977 | | |
| 63 | | |
| PP-976 | | |
| 64 | | |
| PP-972 | | |
| 65 | | |
| PP-971 | | |
| 66 | | |
| PP-968 | | |
| 67 | | |
| PP-959 | | |
| 68 | + | − |
| PP-958 | | |
| 69 | | |
| PP-1281 | | |
| 70 | ++ | |
| PP-1030 | | |

### Example B: Pharmacokinetics of Selected Compounds

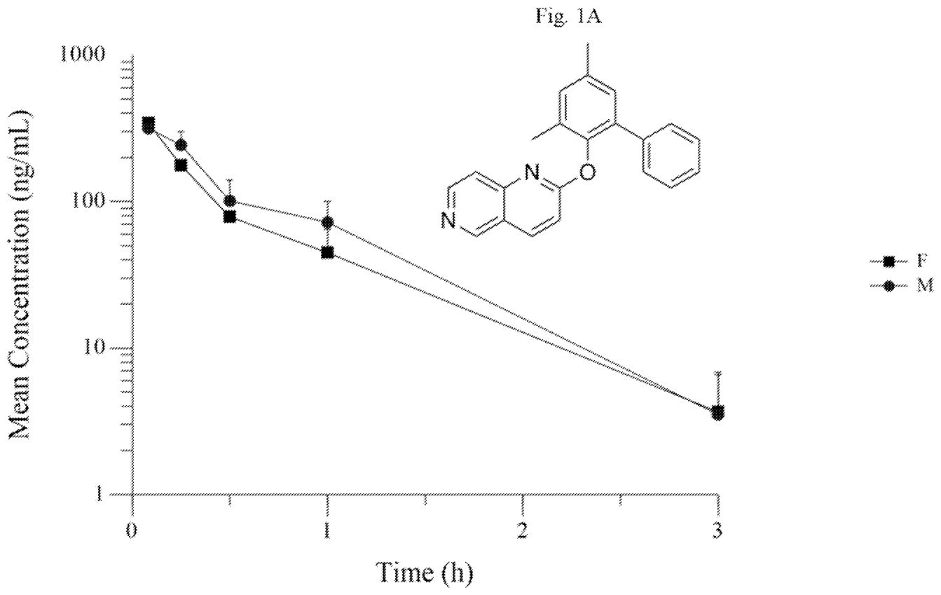

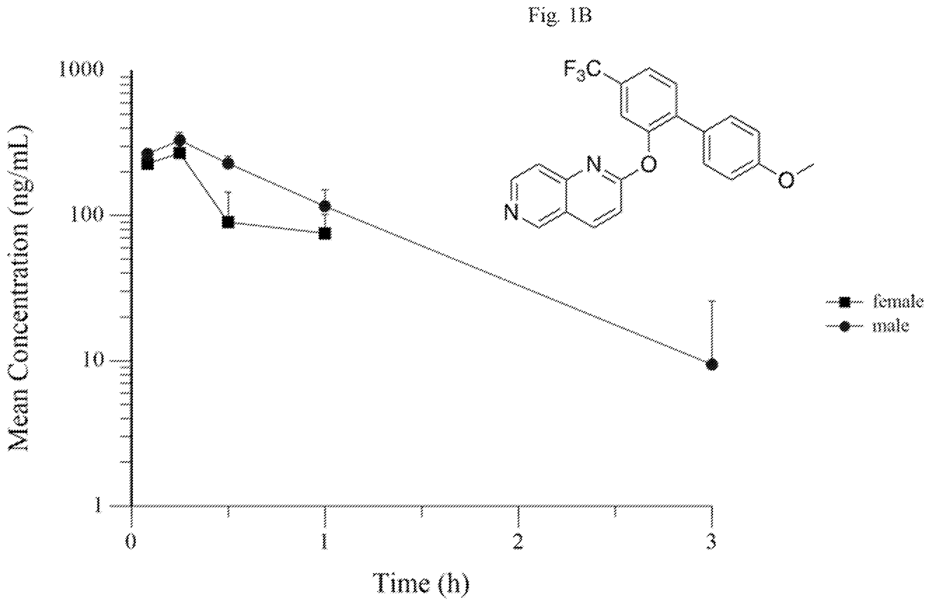

Mice were injected intraperitoneally with (5 mg/kg) of the compound of Example 71 or Example 78 and plasma samples were taken at intervals of 0.83 hr, 0.25 hr, 0.5 hr. 1.0 hr, 3.0 hr, and 6.0 hr post-administration in order to analyze the pharmacokinetic profile of these compounds. The mean plasma content for male and for female mice at these time points post-injection are shown for Example 71 in FIG. 1A and Table 3, and for Example 78, in FIG. 1B and Table 4.

TABLE 3
Plasma Pharmacokinetic Parameters Following 5 mg/kg IP
Administration in Mice

| | | | | | | Cl/F | |
|---|---|---|---|---|---|---|---|
| | Tₘₐₓ | Cₘₐₓ | AUCₗₐₛₜ | AUC_{INF} | Vz/F | (mL/min/ | t_{1/2} |
| Sex | (h) | (ng/mL) | (h*ng/mL) | (h*ng/ml) | (L/kg) | kg) | (h) |
| female | 0.083 | 345 | 169 | 172 | 21.9 | 484 | 0.52 |
| male | 0.083 | 317 | 222 | 225 | 15.2 | 371 | 0.47 |

TABLE 4

| | | | | | | CL/F | |
|---|---|---|---|---|---|---|---|
| | | Tmax | Cmax | AUClast | Vz/F | (mL/min/ | t_{1/2} |
| Compound | Sex | (h) | (ng/mL) | (h*ng/ml) | (L/kg) | kg) | (h) |
| SLUPP- | female | 0.25 | 269 | 137 | | | |
| 1657 | male | 0.25 | 330 | 343 | 11.3 | 238 | 0.55 |

### Prophetic Examples

### Prophetic Example 1: Assessing REV-ERB Agonist Efficacy in a Neurodegenerative Animal Model

The senescence accelerated mouse P8 (SAMP8) mouse model is a well characterized model to study Alzheimer's Disease and for development of drugs to treat AD. REV-ERB agonist SR9009 is effective in reversing the cognitive decline in these mice that is associated with an AD-like pathology. This mouse model will be utilized to evaluate REV-ERB agonist compounds efficacy in reversing cognitive decline. SAMP8 mice (young: 5-month-old or aged: 13-months-old) will be treated with vehicle or REV-ERB agonist for 1 month followed by assessment of memory using three distinct behavioral assays (T-maze for hippocampal task assessment, novel object recognition for nonspatial reference memory assessment, and lever press for operant associative assessment) on separate days. After the final study, mice will be sacrificed and inflammation in the brain will be assessed.

### Prophetic Example 2: Assessing REV-ERB Agonist Efficacy in a Cardiomyopathy Model

To assess efficacy of REV-ERB agonists in cardio-protection using a clinically-relevant disease model, compounds will be tested using the in vivo pressure overload transaortic constriction (TAC) model. TAC will be performed in 9-week old adult male and female wild type C57BL/6 mice. One day after TAC, mice will be randomized based on BW to receive vehicle or REV-ERB agonists. Mice will be evaluated by ECHO (by a blinded sonographer) every two weeks for 6 weeks to monitor the effect on cardiac function and structural remodeling. Detailed analysis will be performed at the end of 6 weeks, including heart weight (biventricular weight normalized to tibia length), wet lung weight (to monitor for pulmonary edema), cardiac fiber staining (WGA), fibrosis (trichrome or picosirius red), myocytes alignment, TUNEL and gene expression (ANF, BNP, ACTA1, etc.). Adult myocytes will be isolated by Langendorff apparatus and myocytes width and length will be measured as indices for concentric vs eccentric hypertrophy.

### Prophetic Example 3: Assessing REV-ERB Agonist Efficacy in an Amyotrophic Lateral Sclerosis Model

The SOD1G93A mouse model is a well-characterized model of ALS. The mice exhibit progression of muscle weakness, involvement of both upper and lower motor neurons, and cellular and molecular changes that are observed in humans. To determine the potential therapeutic effects of REV-ERB agonists, test compounds or placebo will be administered i.p. once a day beginning at postnatal day 30 or 60 through end stage. Initial neuromuscular junction (NMJ) denervation, behavioral deficits, pathological changes in upper cortical spinal motor neurons and lower spinal cord motor neurons, and glia activation are reported to occur within this time frame. Motor behavior will be monitored using leg extension and paw grip endurance assays. NMJ innervation (identified based on size, fluorescent Nissl stain and location within the motor cortex), upper cortical spinal and lower spinal cord motor neuron pathology and number, glial activation (IBA1 and CD68 expression), and astrocyte activation (GFAP positive cells that also express complement C3 as activated) will be analyzed to assess efficacy of REV-ERB agonists compared to placebo.

### Prophetic Example 4: Assessing REV-ERB Agonist Efficacy in a Nonalcoholic Steatohepatitis (NASH) Model

A diet-induced obesity mouse model will be used to replicate the etiology and natural progression of NASH observed in humans. Mice will be fed a diet containing high amounts of trans-fat, fructose, and cholesterol for 6 months to induce NASH, then administered REV-ERB agonist or placebo via i.p. for 30 days while maintaining the NASH diet. Body weight and food intake will be monitored daily. Blood glucose will be quantified weekly with a glucometer, and a final fasting blood glucose collected at experiment termination. Liver will be collected and weighed. Plasma lipid levels and liver health will be analyzed by clinical chemistry and ELISA (liver enzyme levels). qPCR (genes involved in lipogenesis, hepatic steatosis inflammation), Western blot, and immunohistochemistry (to detect signals of fibrosis, steatosis, and inflammation) will be used to assess disease severity and assess the efficacy of REV-ERB agonists at reducing and/or reversing disease.

Embodiments of the disclosure include the following.
1. A compound of the formula:

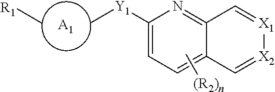

wherein:
   X₁ and X₂ are each independently C or N;
   Y₁ is O, S(O)_{q}, NRₐ, or ⁺NR_{b}R_{c}, wherein
      q is 0, 1, or 2;
      Rₐ, R_{b}, and R_{c} are each independently hydrogen, alkyl_{(C≤6)}, or substituted alkyl_{(C≤6)};
   A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof; or —(CH₂)ₘC(O)—, wherein m is 1, 2, or 3;
   R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
   R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   n is 1, 2, 3, or 4;
or a compound of the formula:

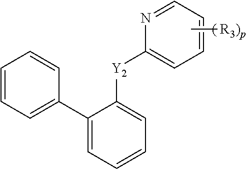

wherein:
   Y₂ is O, S(O)ᵣ, NR_{d}, or ⁺NRₑR_{f}, wherein
      r is 0, 1, or 2;
      R_{d}, Rₑ, and R_{f} are each independently hydrogen, alkyl_{(C≤6)}, or substituted alkyl_{(C≤6)};
   R₃ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   p is 1, 2, or 3
or a pharmaceutically acceptable salt thereof.
2. The compound of Embodiment 1 further defined as:

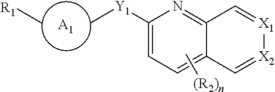

wherein:
   X₁ and X₂ are each independently C or N;
   Y₁ is O, S(O)_{q}, NRₐ, or ⁺NR_{b}R_{c}, wherein
      q is 0, 1, or 2;
      Rₐ, R_{b}, and R_{c} are each independently hydrogen, alkyl_{(C≤6)}, or substituted alkyl_{(C≤6)};
   A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof; or —(CH₂)ₘC(O)—, wherein m is 1, 2, or 3;
   R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
   R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   n is 1, 2, 3, or 4;
or a pharmaceutically acceptable salt thereof.
3. The compound of either Embodiment 1 or Embodiment 2 further defined as:

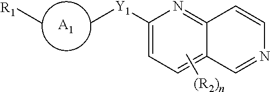

wherein:
   Y₁ is O, S(O)_{q}, NRₐ, or ⁺NR_{b}R_{c}, wherein
      q is 0, 1, or 2;
      Rₐ, R_{b}, and R_{c} are each independently hydrogen, alkyl_{(C≤6)}, or substituted alkyl_{(C≤6)};
   A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof; or —(CH₂)ₘC(O)—, wherein m is 1, 2, or 3;
   R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
   R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   n is 1, 2, 3, or 4;
or a pharmaceutically acceptable salt thereof.
4. The compound according to any one of Embodiments 1-3 further defined as:

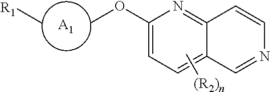

wherein:
   A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof;
   R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
   R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   n is 1, 2, 3, or 4;
or a pharmaceutically acceptable salt thereof.
5. The compound according to any one of claims 1-3 further defined as:

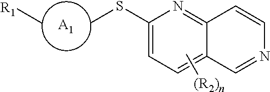

wherein:
   A₁ is —(CH₂)ₘC(O)—, wherein m is 1, 2, or 3;
   R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
   R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   n is 1, 2, 3, or 4;
or a pharmaceutically acceptable salt thereof.
6. The compound of either Embodiment 1 or Embodiment 2 further defined as:

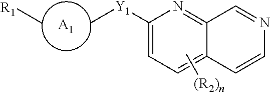

wherein:
   Y₁ is O, S(O)_{q}, NRₐ, or ⁺NR_{b}R_{c}, wherein
      q is 0, 1, or 2;
      Rₐ, R_{b}, and R_{c} are each independently hydrogen, alkyl_{(C≤6)}, or substituted alkyl_{(C≤6)};
   A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof; or —(CH₂)ₘC(O)—, wherein m is 1, 2, or 3;
   R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
   R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   n is 1, 2, 3, or 4;
or a pharmaceutically acceptable salt thereof.
7. The compound according to any one of Embodiments 1, 2, and 6 further defined as:

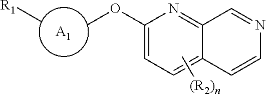

wherein:
   A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof;
   R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
   R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   n is 1, 2, 3, or 4;
or a pharmaceutically acceptable salt thereof.
8. The compound according to any one of Embodiments 1, 2, and 6 further defined as:

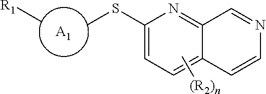

wherein:
   A₁ is —(CH₂)ₘC(O)—, wherein m is 1, 2, or 3;
   R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
   R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
   n is 1, 2, 3, or 4;
or a pharmaceutically acceptable salt thereof.
9. The compound of either Embodiment 1 or Embodiment 2, wherein X₁ is C.
10. The compound of either Embodiment 1 or Embodiment 2, wherein X₁ is N.
11. The compound according to any one of Embodiments 1, 2, 9, and 10, wherein X₂ is C.
12. The compound according to any one of Embodiments 1, 2, 9, and 10, wherein X₂ is N.
13. The compound according to any one of Embodiments 1, 2, 3, 6, and 9-12, wherein Y₁ is O.
14. The compound according to any one of Embodiments 1, 2, 3, 6, and 9-12, wherein Y₁ is S.
15. The compound according to any one of Embodiments 1, 2, 3, 6, and 9-12, wherein Y₁ is NRₐ.
16. The compound of Embodiment 15, wherein Rₐ is hydrogen.
17. The compound according to any one of Embodiments 1, 2, 3, 6, and 9-12, wherein Y₁ is ⁺NR_{b}R_{c}.
18. The compound of Embodiment 17, wherein R_{b} is methyl.
19. The compound of either Embodiment 17 or Embodiment 18, wherein R_{c} is methyl.
20. The compound according to any one of Embodiments 1-4, 6, 7, and 9-19, wherein A₁ is arenediyl_{(C≤18)} or substituted arenediyl_{(C≤18)}.
21. The compound of Embodiment 20, wherein A₁ is arenediyl_{(C≤18)}.
22. The compound of Embodiment 20, wherein A₁ is substituted arenediyl_{(C≤18)}.
23. The compound according to any one of Embodiments 1-4, 6, 7, and 9-19, wherein A₁ is cycloalkanediyl_{(C≤18)} or substituted cycloalkanediyl_{(C≤18)}.
24. The compound of Embodiment 23, wherein A₁ is cycloalkanediyl_{(C≤18)}.
25. The compound of Embodiment 23, wherein A₁ is substituted cycloalkanediyl_{(C≤18)}.
26. The compound according to any one of Embodiments 1-4, 6, 7, and 9-19, wherein A₁ is aralkenediyl_{(C≤18)} or substituted aralkenediyl_{(C≤18)}.
27. The compound of Embodiment 26, wherein A₁ is aralkenediyl_{(C≤18)}.
28. The compound of Embodiment 26, wherein A₁ is substituted aralkenediyl_{(C≤18)}.
29. The compound according to any one of Embodiments 1-3, 5, and 8-19, wherein A₁ is —(CH₂)ₘC(O)—.
30. The compound of Embodiment 29, wherein m is 1.
31. The compound according to any one of Embodiments 1-30, wherein R₁ is hydrogen.
32. The compound according to any one of Embodiments 1-30, wherein R₁ is halo.
33. The compound of Embodiment 32, wherein R₁ is fluoro or bromo.
34. The compound according to any one of Embodiments 1-30, wherein R₁ is hydroxy.
35. The compound according to any one of Embodiments 1-30, wherein R₁ is alkyl_{(C≤12)} or substituted alkyl_{(C≤12)}.
36. The compound of Embodiment 35, wherein R₁ is alkyl_{(C≤12)}.
37. The compound of Embodiment 35, wherein R₁ is substituted alkyl_{(C≤12)}.
38. The compound according to any one of Embodiments 1-30, wherein R₁ is cycloalkyl_{(C≤12)} or substituted cycloalkyl_{(C≤12)}.
39. The compound of Embodiment 38, wherein R₁ is cycloalkyl_{(C≤12)}.
40. The compound of Embodiment 39, wherein R₁ is substituted cycloalkyl_{(C≤12)}.
41. The compound according to any one of Embodiments 1-30, wherein R₁ is aryl_{(C≤12)} or substituted aryl_{(C≤12)}.
42. The compound of Embodiment 41, wherein R₁ is aryl_{(C≤12)}.
43. The compound of Embodiment 41, wherein R₁ is substituted aryl_{(C≤12)}.
44. The compound according to any one of Embodiments 1-30, wherein R₁ is heteroaryl_{(C≤12)} or substituted heteroaryl_{(C≤12)}.
45. The compound of Embodiment 44, wherein R₁ is heteroaryl_{(C≤12)}.
46. The compound of Embodiment 44, wherein R₁ is substituted heteroaryl_{(C≤12)}.
47. The compound according to any one of Embodiments 1-30, wherein R₁ is heterocycloalkyl_{(C≤12)} or substituted heterocycloalkyl_{(C≤12)}.
48. The compound of Embodiment 47, wherein R₁ is heterocycloalkyl_{(C≤12)}.
49. The compound of Embodiment 47, wherein R₁ is substituted heterocycloalkyl_{(C≤12)}.
50. The compound according to any one of Embodiments 1-30, wherein R₁ is alkoxy_{(C≤12)} or substituted alkoxy_{(C≤12)}.
51. The compound of Embodiment 50, wherein R₁ is alkoxy_{(C≤12)}.
52. The compound of Embodiment 51, wherein R₁ is substituted alkoxy_{(C≤12)}.
53. The compound according to any one of Embodiments 1-30, wherein R₁ is aralkoxy_{(C≤12)} or substituted aralkoxy_{(C≤12)}.
54. The compound of Embodiment 53, wherein R₁ is aralkoxy_{(C≤12)}.
55. The compound of Embodiment 53, wherein R₁ is substituted aralkoxy_{(C≤12)}.
56. The compound according to any one of Embodiments 1-30, wherein R₁ is aralkylamino_{(C≤12)} or substituted aralkylamino_{(C≤12)}.
57. The compound of Embodiment 56, wherein R₁ is aralkylamino_{(C≤12)}.
58. The compound of Embodiment 56, wherein R₁ is substituted aralkylamino_{(C≤12)}.
59. The compound according to any one of Embodiments 1-30, wherein R₁ is -heterocycloalkanediyl_{(C≤8)}-heteroaryl_{(C≤12)} or substituted -heterocycloalkanediyl_{(C≤8)}-heteroaryl_{(C≤12)}.
60. The compound of Embodiment 59, wherein R¹ is -heterocycloalkanediyl_{(C≤8)}-heteroaryl_{(C≤12)}.
61. The compound of Embodiment 59, wherein R₁ is substituted -heterocycloalkanediyl_{(C≤8)}-heteroaryl_{(C≤12)}.
62. The compound according to any one of Embodiments 1-61, wherein R₂ is hydrogen.
63. The compound according to any one of Embodiments 1-61, wherein R₂ is alkyl_{(C≤8)} or substituted alkyl_{(C≤8)}.
64. The compound of Embodiment 63, wherein R₂ is alkyl_{(C≤8)}.
65. The compound of Embodiment 63, wherein R₂ is substituted alkyl_{(C≤8)}.
66. The compound according to any one of Embodiments 1-61, wherein R₂ is alkoxy_{(C≤8)} or substituted alkoxy_{(C≤8)}.
67. The compound of Embodiment 66, wherein R₂ is alkoxy_{(C≤8)}.
68. The compound of Embodiment 66, wherein R₂ is substituted alkoxy_{(C≤8)}.
69. The compound according to any one of Embodiments 1-68, wherein n is 1 or 2.
70. The compound of Embodiment 69, wherein n is 1.
71. The compound of Embodiment 69, wherein n is 2.
72. The compound of Embodiment 1, wherein the compound is further defined as:

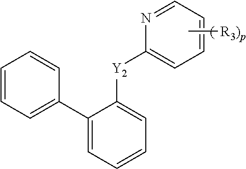

wherein:
   Y₂ is O, S(O)ᵣ, NR_{d}, or ⁺NRₑR_{f}, wherein
      r is 0, 1, or 2;
      R_{d}, Rₑ, and R_{f} are each independently hydrogen, alkyl_{(C≤6)}, or substituted alkyl_{(C≤6)};
   R₃ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤|8)}, amido_{(C≤8)}, alkylamino_{(C≤8)}, dialkylamino_{(C≤8)}, or a substituted version thereof; and
   p is 1, 2, or 3
or a pharmaceutically acceptable salt thereof.
73. The compound of either Embodiment 1 or Embodiment 72, wherein Y₁ is O.
74. The compound according to any one of Embodiments 1, 72, and 73, wherein R₃ is cyano.
75. The compound according to any one of Embodiments 1, 72, and 73, wherein R₃ is carboxy.
76. The compound according to any one of Embodiments 1-75 further defined as:

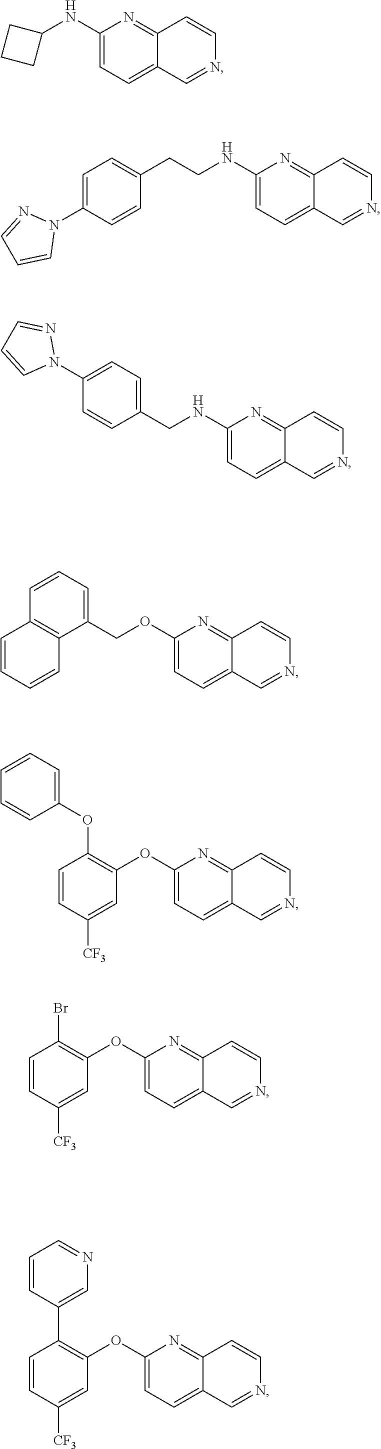

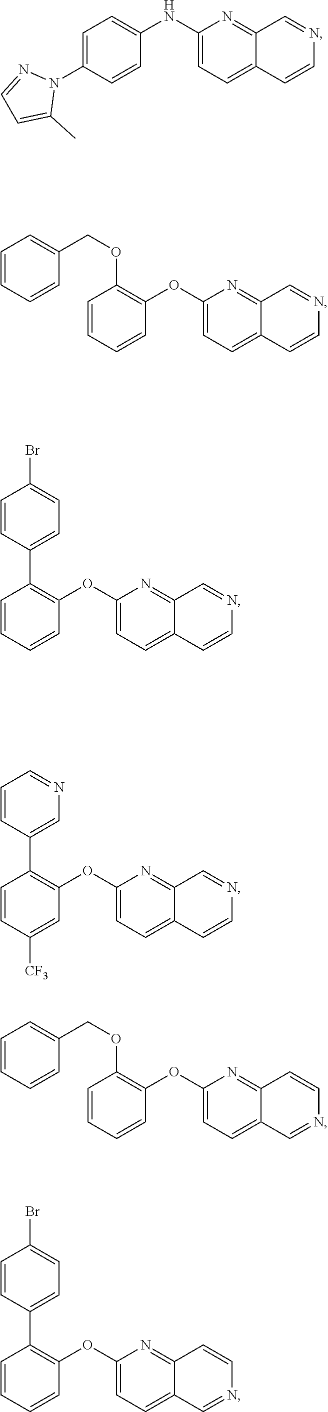 [SMILES: Cc1ccnn1-c1ccc(Nc2ccc3ccncc3n2)cc1 ; c1ccc(COc2ccccc2Oc2ccc3ccncc3n2)cc1 ; Brc1ccc(-c2ccccc2Oc2ccc3ccncc3n2)cc1 ; FC(F)(F)c1ccc(-c2cccnc2)c(Oc2ccc3ccncc3n2)c1 ; c1ccc(COc2ccccc2Oc2ccc3cnccc3n2)cc1 ; Brc1ccc(-c2ccccc2Oc2ccc3cnccc3n2)cc1]

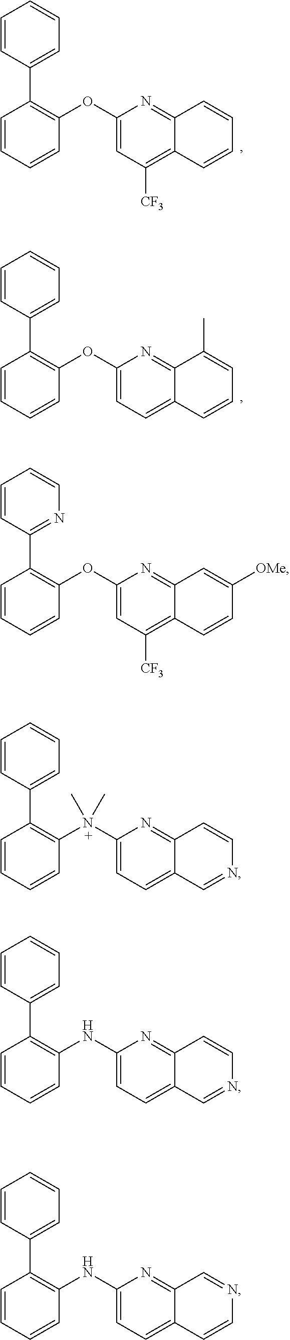

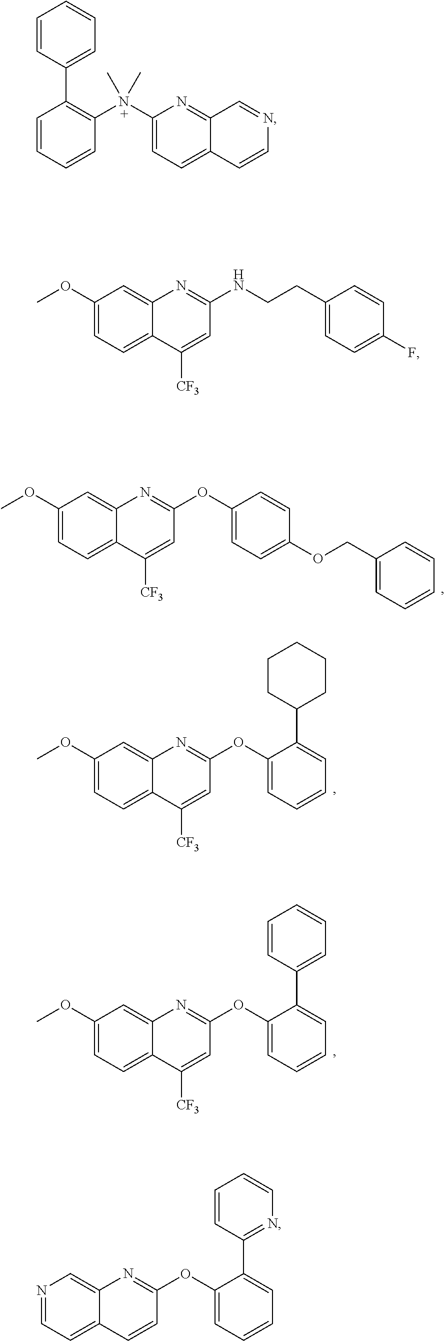

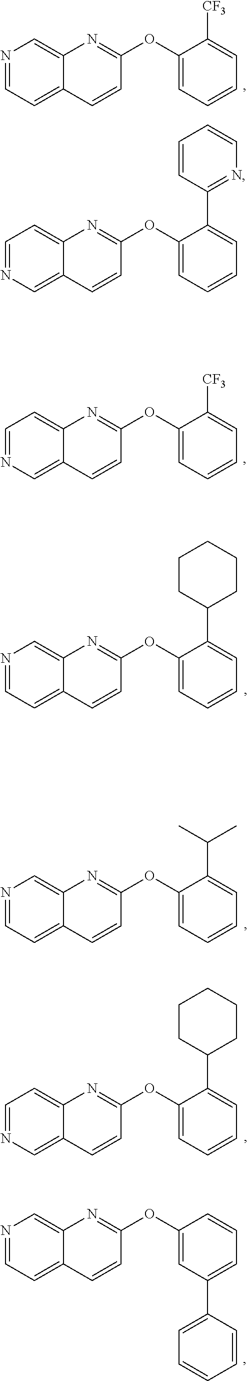

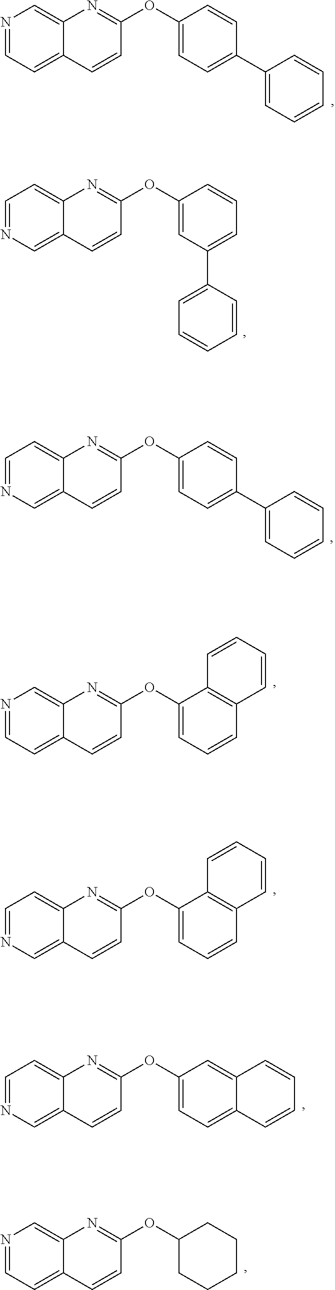

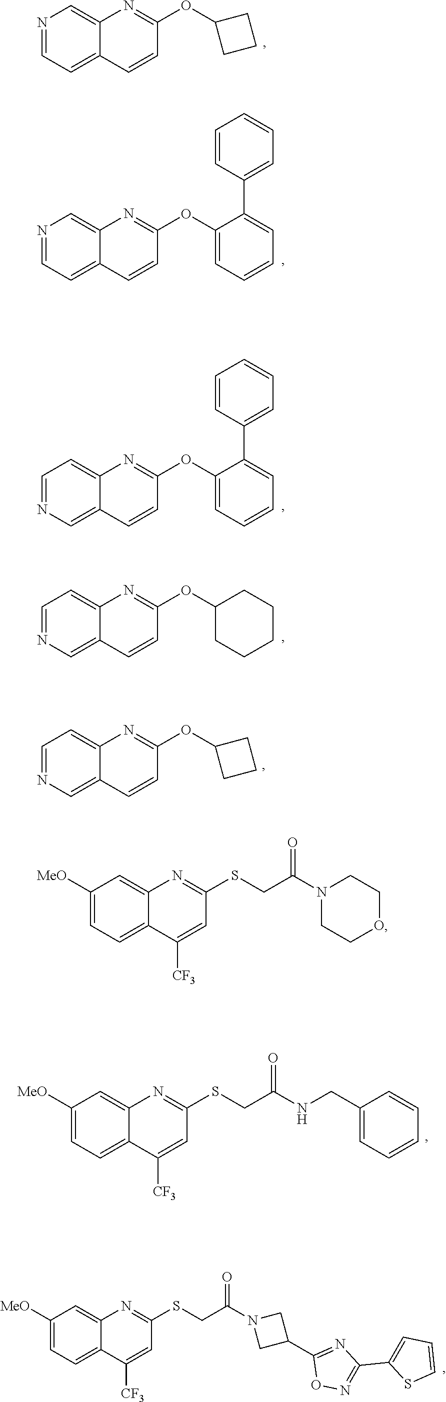

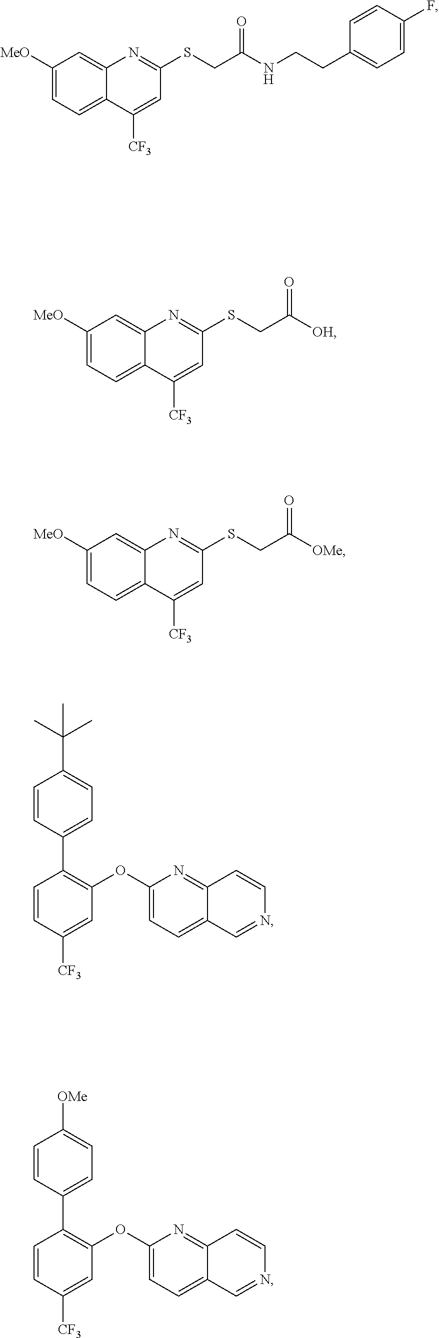 [SMILES: COc1ccc2c(C(F)(F)F)cc(SCC(=O)NCCc3ccc(F)cc3)nc2c1 ; COc1ccc2c(C(F)(F)F)cc(SCC(=O)O)nc2c1 ; COC(=O)CSc1cc(C(F)(F)F)c2ccc(OC)cc2n1 ; CC(C)(C)c1ccc(-c2ccc(C(F)(F)F)cc2Oc2ccc3cnccc3n2)cc1 ; COc1ccc(-c2ccc(C(F)(F)F)cc2Oc2ccc3cnccc3n2)cc1]

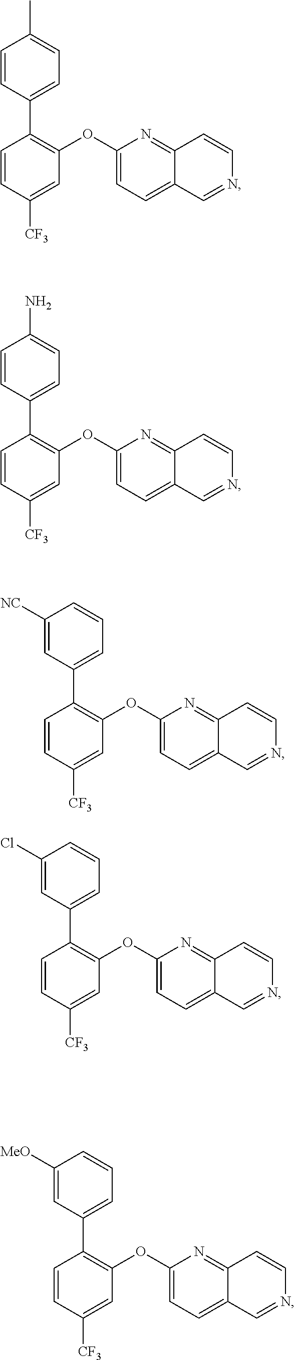

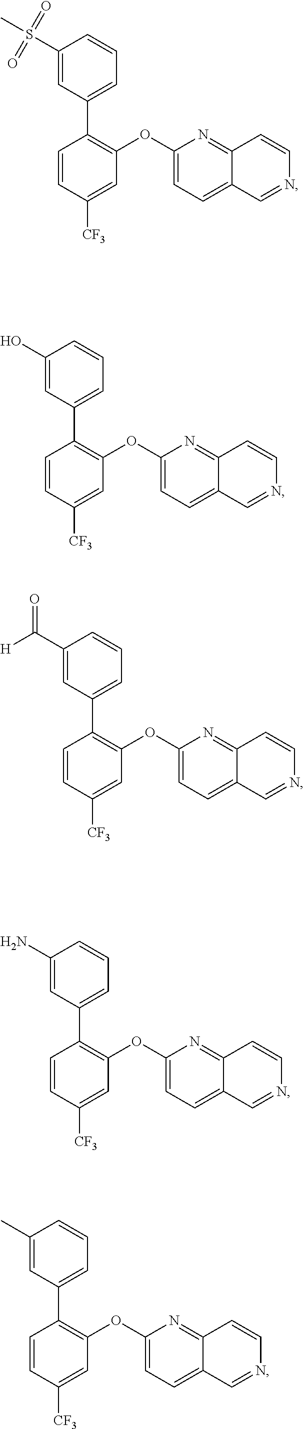 [SMILES: CS(=O)(=O)c1cccc(-c2ccc(C(F)(F)F)cc2Oc2ccc3cnccc3n2)c1 ; Oc1cccc(-c2ccc(C(F)(F)F)cc2Oc2ccc3cnccc3n2)c1 ; O=Cc1cccc(-c2ccc(C(F)(F)F)cc2Oc2ccc3cnccc3n2)c1 ; Nc1cccc(-c2ccc(C(F)(F)F)cc2Oc2ccc3cnccc3n2)c1 ; Cc1cccc(-c2ccc(C(F)(F)F)cc2Oc2ccc3cnccc3n2)c1]

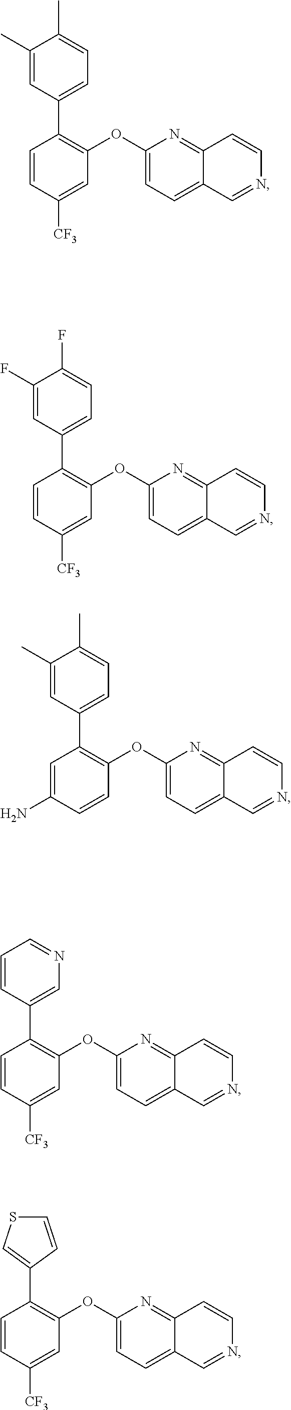 [SMILES: Cc1ccc(-c2ccc(C(F)(F)F)cc2Oc2ccc3cnccc3n2)cc1C ; Fc1ccc(-c2ccc(C(F)(F)F)cc2Oc2ccc3cnccc3n2)cc1F ; Cc1ccc(-c2cc(N)ccc2Oc2ccc3cnccc3n2)cc1C ; FC(F)(F)c1ccc(-c2cccnc2)c(Oc2ccc3cnccc3n2)c1 ; FC(F)(F)c1ccc(-c2ccsc2)c(Oc2ccc3cnccc3n2)c1]

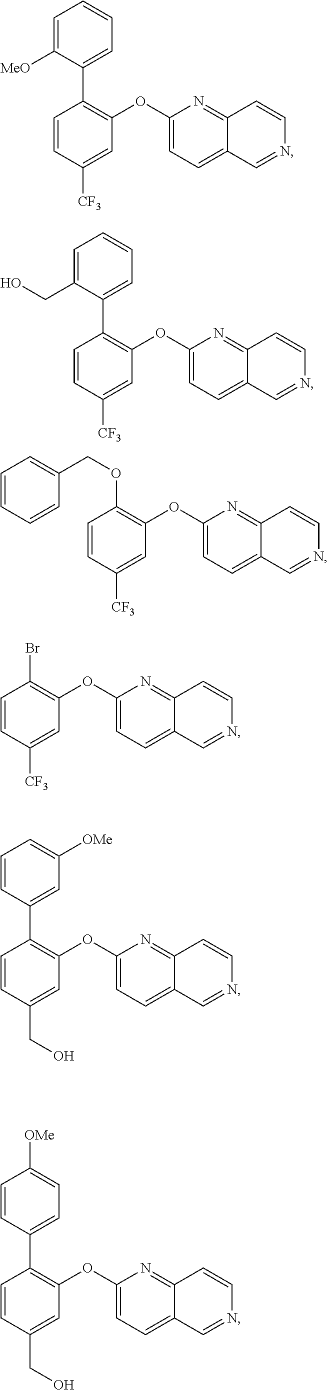

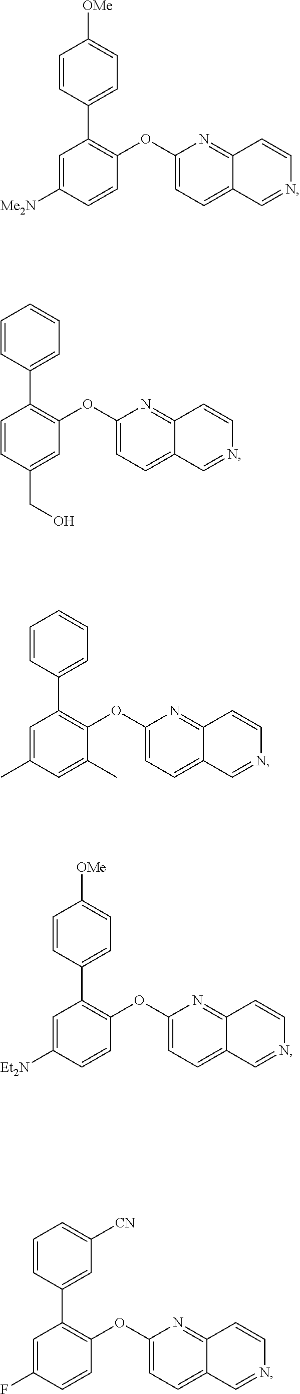

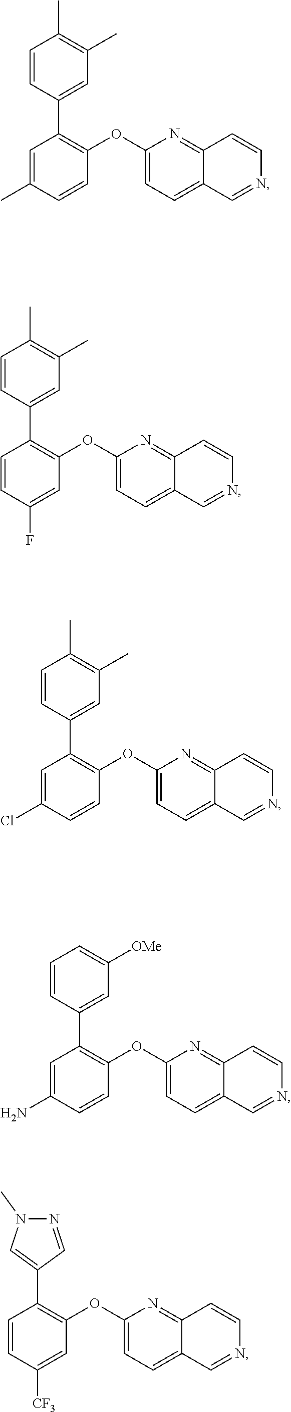 [SMILES: Cc1ccc(Oc2ccc3cnccc3n2)c(-c2ccc(C)c(C)c2)c1 ; Cc1ccc(-c2ccc(F)cc2Oc2ccc3cnccc3n2)cc1C ; Cc1ccc(-c2cc(Cl)ccc2Oc2ccc3cnccc3n2)cc1C ; COc1cccc(-c2cc(N)ccc2Oc2ccc3cnccc3n2)c1 ; Cn1cc(-c2ccc(C(F)(F)F)cc2Oc2ccc3cnccc3n2)cn1]

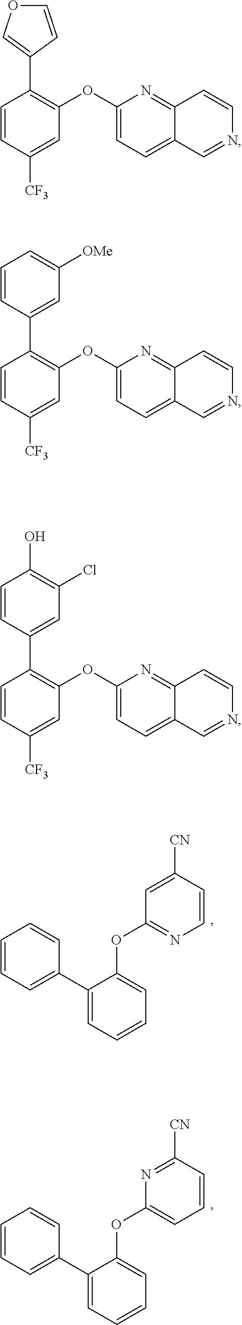 [SMILES: FC(F)(F)c1ccc(-c2ccoc2)c(Oc2ccc3cnccc3n2)c1 ; COc1cccc(-c2ccc(C(F)(F)F)cc2Oc2ccc3cnccc3n2)c1 ; Oc1ccc(-c2ccc(C(F)(F)F)cc2Oc2ccc3cnccc3n2)cc1Cl ; N#Cc1ccnc(Oc2ccccc2-c2ccccc2)c1 ; N#Cc1cccc(Oc2ccccc2-c2ccccc2)n1]

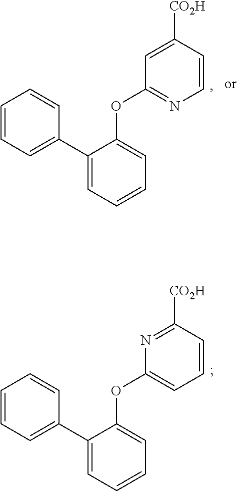 [SMILES: O=C(O)c1ccnc(Oc2ccccc2-c2ccccc2)c1 ; O=C(O)c1cccc(Oc2ccccc2-c2ccccc2)n1]

or a pharmaceutically acceptable salt thereof.
77. A compound of Formula (VIII):

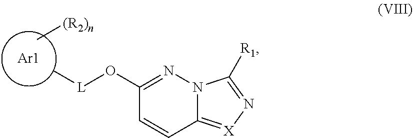

wherein:
   X is N or CH;
   R₁ is H, halogen, or C₁-C₆ alkyl;
   L is a bond or C₁-C₂ alkylenyl;
   Ar1 is phenyl or naphthalenyl;
   R₂ is selected from the group consisting of H, C₁-C₆ alkyl, C₁-C₆ alkoxy, phenyl, -L₂-R₃, and —O-L₂-R₃, wherein
      L₂ is C₁-C₂ alkylenyl, and
      R₃ is selected from the group consisting of C₆-C₁₀ aryl and C₃-C₆ cycloalkyl, wherein the C₆-C₁₀ aryl is unsubstituted or substituted with 1-3 groups which may be the same or different selected from the group consisting of halogen, C₁-C₆ alkyl, C₁-C₆ haloalkyl, C₁-C₆ alkoxy, and phenyl;
   n is an integer selected from 0, 1, and 2,
or a pharmaceutically acceptable salt thereof.
78. A compound of the formula:

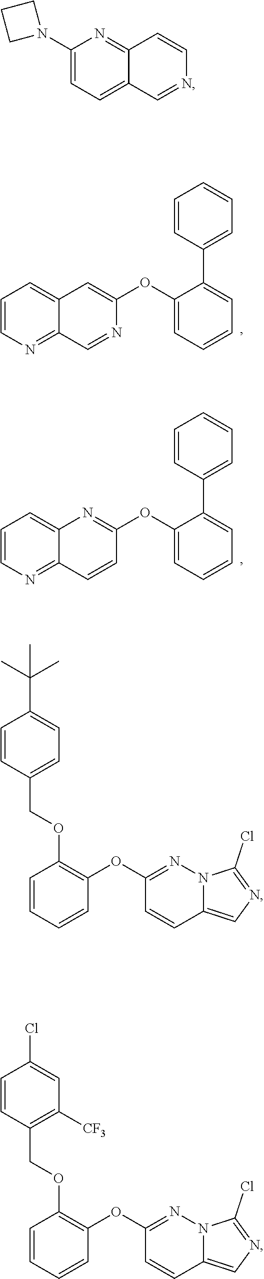 [SMILES: c1cc2nc(N3CCC3)ccc2cn1 ; c1ccc(-c2ccccc2Oc2cc3cccnc3cn2)cc1 ; c1ccc(-c2ccccc2Oc2ccc3ncccc3n2)cc1 ; CC(C)(C)c1ccc(COc2ccccc2Oc2ccc3cnc(Cl)n3n2)cc1 ; FC(F)(F)c1cc(Cl)ccc1COc1ccccc1Oc1ccc2cnc(Cl)n2n1]

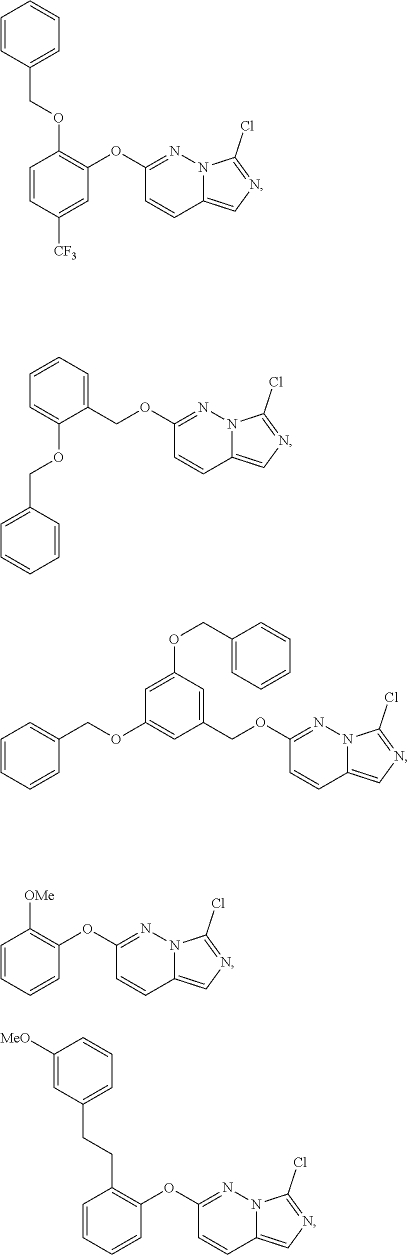

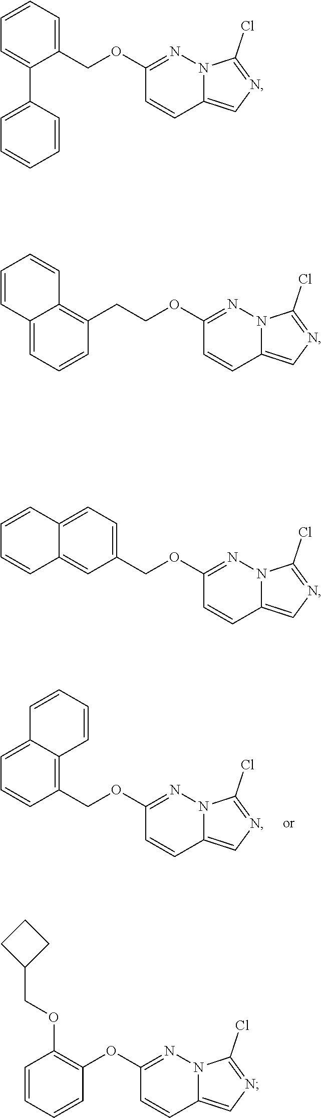 [SMILES: Clc1ncc2ccc(OCc3ccccc3-c3ccccc3)nn12 ; Clc1ncc2ccc(OCCc3cccc4ccccc34)nn12 ; Clc1ncc2ccc(OCc3ccc4ccccc4c3)nn12 ; Clc1ncc2ccc(OCc3cccc4ccccc34)nn12 ; Clc1ncc2ccc(Oc3ccccc3OCC3CCC3)nn12]

or a pharmaceutically acceptable salt thereof.
79. A pharmaceutical composition comprising:
   (A) a compound according to any one of Embodiments 1-78; and
   (B) an excipient.
80. The pharmaceutical composition of Embodiment 79, wherein the pharmaceutical composition has been formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.
81. The pharmaceutical composition of Embodiment 78, wherein the pharmaceutical composition is formulated as a unit dose.
82. A method of treating a disease or disorder in a patient using a compound or pharmaceutical composition according to any one of Embodiments 1-81 comprising administering to the patient in need thereof a therapeutically effective amount of the compound or pharmaceutical composition.
83. The method of Embodiment 82, wherein the disease or disorder is a neurological disease.
84. The method of Embodiment 83, wherein the neurological disease is an anxiety disorder.
85. The method of Embodiment 82, wherein the disease or disorder is an autoimmune disorder.
86. The method of Embodiment 82, wherein the disease or disorder is a muscular disorder.
87. The method of Embodiment 86, wherein the muscular disorder is sarcopenia.
88. The method according to any one of Embodiments 82-87, wherein the method further comprises administering a second therapeutic agent.
89. The method according to any one of Embodiments 82-88, wherein the method comprises administering the compound once.
90. The method according to any one of Embodiments 82-88, wherein the method comprises administering the compound two or more times.
91. A method of modulating the activity of a nuclear receptor comprising contacting the nuclear receptor with an effective amount of the compound according to any one of Embodiments 1-81.
92. The method of Embodiment 91, wherein the nuclear receptor is a rev-erb nuclear receptor.
93. The method of either Embodiment 91 or Embodiment 92, wherein the method is performed in vitro.
94. The method of either Embodiment 91 or Embodiment 92, wherein the method is performed ex vivo.
95. The method of either Embodiment 91 or Embodiment 92, wherein the method is performed in vivo.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1.-8. (canceled)

9. A compound, or a pharmaceutically acceptable salt, hydrate or solvate thereof, of Formula (III): 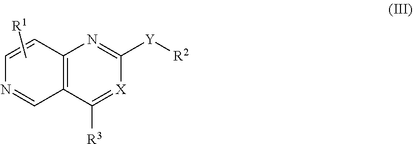
X is CR⁴ or N;
Y is a direct bond, O, S, SO, SO₂, CH₂ or NR⁵ or N⁺(R⁵)₂;
R¹ is H, halogen, NHR⁵, C₁-C₅ haloalkyl, C₁-C₅ alkoxy, or O—C₃-C₇ cycloalkyl; or C₁-C₆ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇-cycloalkyl; or G²;
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
each R⁹ and R¹⁰ is independently H, C₁-C₆ alkyl, halogen, C₁-C₅ haloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R⁹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R⁹ is independently H, C₁-C₅ alkyl, C₁-C₅ haloalkyl, or C₁-C₅ alkyl substituted with NR⁵R⁵ or OR⁵.

10. The compound according to claim 9 wherein
Y is O, S, SO, SO₂, NH or N⁺(CH₃)₂;
R¹ is H or C₁-C₅ alkyl;
R² is C₁-C₅-alkyl or C₃-C₆-cycloalkyl; or phenyl or naphthalenyl, each optionally substituted with up to 5 substituents independently selected from R⁶ and R⁷;
R³ is H or C₁-C₅ alkyl; and
R⁴ is H or C₁-C₅ alkyl.

11. The compound according to claim 10 wherein
Y is O;
R¹ is H;
R³ is H; and
R⁴ is H.

12. The compound according to claim 9 wherein
Y is O;
R¹ is H;
R² is phenyl substituted with one R⁶ and optionally substituted with up to 2 substituents independently selected from R⁷;
R³ is H;
R⁴ is H;
R⁶ is G²;
G² is phenyl optionally substituted with up to 3 substituents independently selected from R⁷; or a 5- to 6-membered fully unsaturated heterocyclic ring containing ring members selected from carbon atoms and 1 to 4 heteroatoms independently selected from up to 2 O, up to 2 S and up to 4 N atoms, optionally substituted with up to 3 substituents independently selected from R⁷ on carbon atom ring members and selected from R⁹ on nitrogen atom ring members;
each R⁷ is independently H, C₁-C₆ alkyl, halogen, NHR⁵ or C₁-C₅ haloalkyl; and
each R⁹ is independently H or methyl.

13. A compound, or a pharmaceutically acceptable salt, hydrate or solvate thereof, of Formula (IV): 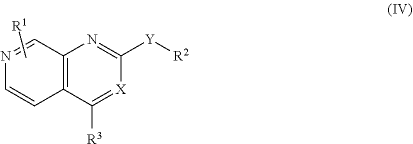
X is CR⁴ or N;
Y is a direct bond, O, S, SO, SO₂, CH₂ or NR⁵ or N⁺(R⁵)₂;
R¹ is H, halogen, NHR⁵, C₁-C₅ haloalkyl, C₁-C₅ alkoxy, or O—C₃-C₇ cycloalkyl; or C₁-C₆ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇-cycloalkyl; or G²;
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
each R⁹ and R¹⁰ is independently H, C₁-C₆ alkyl, halogen, C₁-C₅ haloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R¹¹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R⁹ is independently H, C₁-C₅ alkyl, C₁-C₅ haloalkyl, or C₁-C₅ alkyl substituted with NR⁵R⁵ or OR⁵.

14.-16. (canceled)

17. A compound, or a pharmaceutically acceptable salt, hydrate or solvate thereof, of Formula (V): 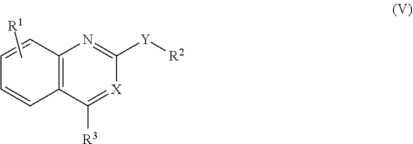
wherein
X is CR⁴ or N;
Y is a direct bond, O, S, SO, SO₂, CH₂ or NR⁵ or N⁺(R⁵)₂;
R¹ is H, halogen, NHR⁵, C₁-C₅ haloalkyl, C₁-C₅ alkoxy, or O—C₃-C₇ cycloalkyl; or C₁-C₆ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇ cycloalkyl; or G²;
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
each R⁹ and R¹⁰ is independently H, C₁-C₆ alkyl, halogen, C₁-C₅ haloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R⁹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R⁹ is independently H, C₁-C₅ alkyl, C₁-C₅ haloalkyl, or C₁-C₅ alkyl substituted with NR⁵R⁵ or OR⁵.

18.-20. (canceled)

21. A compound, or a pharmaceutically acceptable salt, hydrate or solvate thereof, of Formula (VI): 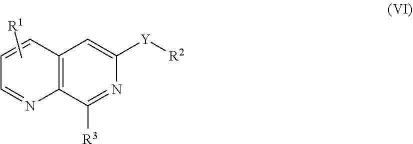
wherein
X is CR⁴ or N;
Y is a direct bond, O, S, SO, SO₂, CH₂ or NR⁵ or N⁺(R⁵)₂;
R¹ is H, halogen, NHR⁵, C₁-C₅ haloalkyl, C₁-C₅ alkoxy, or O—C₃-C₇ cycloalkyl; or C₁-C₆ alkyl or C₃-C₇ cycloalkyl, each optionally substituted with one or more groups selected from halogen, hydroxy, C₁-C₅ alkoxy or NR⁵R⁵;
R² is C₁-C₅-alkyl, C₃-C₆-cycloalkyl or C₁-C₄ alkylene-COR⁸; or phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members;
R³ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
R⁴ is H, C₁-C₅ alkyl, C₁-C₅ haloalkyl, halogen, C₁-C₅ alkoxy, NR⁵R⁵, or C₁-C₅ alkyl substituted with NR⁵R⁵ or OR⁵;
each R⁵ is independently H, C₁-C₅ alkyl or C₃-C₇ cycloalkyl;
each R⁶ is independently H, C₁-C₆ alkyl, C₁-C₄ alkyl-G¹, OC₁-C₄ alkyl-G¹ or C₃-C₇ cycloalkyl; or G²;
each R⁷ is independently H, C₁-C₆ alkyl, halogen, NHR⁵, C₁-C₅ haloalkyl, C₃-C₇ cycloalkyl, cyano, C₁-C₅ alkoxy, or O—C₃-C₇ cycloalkyl;
R⁸ is G³, O—C₁-C₄ alkylene-G³ or NH—C₁-C₄ alkylene-G³;
each G¹ is independently phenyl or naphthalenyl, each optionally substituted with up to 5 substituents independently selected from R⁶ and R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁶ and R⁷ on carbon atom ring members and selected from R¹¹ on nitrogen atom ring members;
each G² is phenyl or naphthalenyl, each optionally substituted with up to 5 substituents independently selected from R⁷ or G³; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ or G³ on carbon atom ring members and selected from R⁹ on nitrogen atom ring members;
G³ is phenyl or naphthalenyl, each optionally substituted with up to 5 substituents independently selected R⁷; or a 5- to 6-membered saturated, partially unsaturated, or fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, each ring or ring system optionally substituted with up to 5 substituents independently selected from R⁷ on carbon atom ring members and selected from R⁹ on nitrogen atom ring members; and
each R⁹ is independently H, C₁-C₅ alkyl, C₁-C₅ haloalkyl, or C₁-C₅ alkyl substituted with NR⁵R⁵ or OR⁵.

22.-26. (canceled)

27. A compound of Formula (VII): 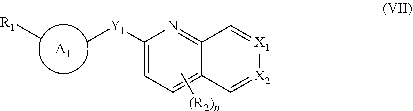
wherein:
X₁ and X₂ are each independently C or N;
Y₁ is O, S(O)_{q}, NRₐ, or ⁺NR_{b}R_{c}, wherein
q is 0, 1, or 2;
Rₐ, R_{b}, and R_{c} are each independently hydrogen, alkyl_{(C≤6)}, or substituted alkyl_{(C≤6)};
A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof; or —(CH₂)ₘC(O)—, wherein m is 1, 2, or 3;
R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
n is 1, 2, 3, or 4;
or a compound of the formula: 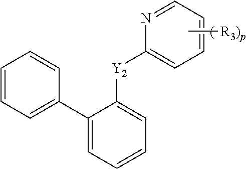
wherein:
Y₂ is O, S(O)ᵣ, NRₐ, or ⁺NRₑR_{f}, wherein
r is 0, 1, or 2;
R_{d}, Rₑ, and R_{f} are each independently hydrogen, alkyl_{(C≤6)}, or substituted alkyl_{(C≤6)};
R₃ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
p is 1, 2, or 3
or a pharmaceutically acceptable salt thereof.

28. The compound of claim 27 further defined as: 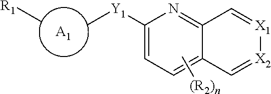
wherein:
X₁ and X₂ are each independently C or N;
Y₁ is O, S(O)_{q}, NRₐ, or ⁺NR_{b}R_{c}, wherein
q is 0, 1, or 2;
Rₐ, R_{b}, and R_{c} are each independently hydrogen, alkyl_{(C≤6)}, or substituted alkyl_{(C≤6)};
A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof; or —(CH₂)ₘC(O)—, wherein m is 1, 2, or 3;
R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
n is 1, 2, 3, or 4;
or a pharmaceutically acceptable salt thereof.

29. The compound of claim 27 further defined as: 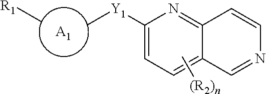
wherein:
Y₁ is O, S(O)_{q}, NRₐ, or ⁺NR_{b}R_{c}, wherein
q is 0, 1, or 2;
Rₐ, R_{b}, and R_{c} are each independently hydrogen, alkyl_{(C≤6)}, or substituted alkyl_{(C≤6)};
A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof; or —(CH₂)ₘC(O)—, wherein m is 1, 2, or 3;
R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
n is 1, 2, 3, or 4;
or a pharmaceutically acceptable salt thereof.

30. The compound according to claim 27, further defined as: 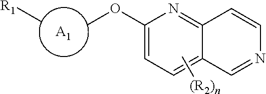
wherein:
A₁ is cycloalkanediyl_{(C≤18)}, heterocycloalkanediyl_{(C≤18)}, arenediyl_{(C≤18)}, heteroarenediyl_{(C≤18)}, aralkenediyl_{(C≤18)}, or a substituted version thereof;
R₁ is hydrogen, amino, cyano, halo, hydroxy, sulfonyl, or alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, alkoxy_{(C≤12)}, aralkoxy_{(C≤12)}, alkylamino_{(C≤12)}, dialkylamino_{(C≤12)}, aralkylamino_{(C≤12)}, -heterocycloalkanediyl_{(C≤8)}-heteroaryl_{(C≤12)}, or a substituted version thereof;
R₂ is hydrogen, amino, carboxy, cyano, halo, hydroxy, alkyl_{(C≤8)}, cycloalkyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, amido_{(C≤8)}, alkylamino_{(C≤8)}, dialkylamino_{(C≤8)}, or a substituted version thereof; and
n is 1, 2, 3, or 4;
or a pharmaceutically acceptable salt thereof.

31.-34. (canceled)

35. The compound of claim 27, wherein X₁ is C.

36.-37. (canceled)

38. The compound according to claim 27, wherein X₂ is N.

39. The compound according to claim 27, wherein Y₁ is O.

40.-45. (canceled)

46. The compound according to claim 27, wherein A₁ is arenediyl_{(C≤18)} or substituted arenediyl_{(C≤18)}.

47.-57. (canceled)

58. The compound according to claim 27, wherein R₁ is hydrogen.

59.-67. (canceled)

68. The compound according to claim 27, wherein R₁ is aryl_{(C≤12)} or substituted aryl_{(C≤12)}.

69.-88. (canceled)

89. The compound according to claim 27, wherein R₂ is hydrogen.

90.-95. (canceled)

96. The compound according to claim 27, wherein n is 1 or 2.

97.-102. (canceled)

103. The compound according to claim 27, further defined as: 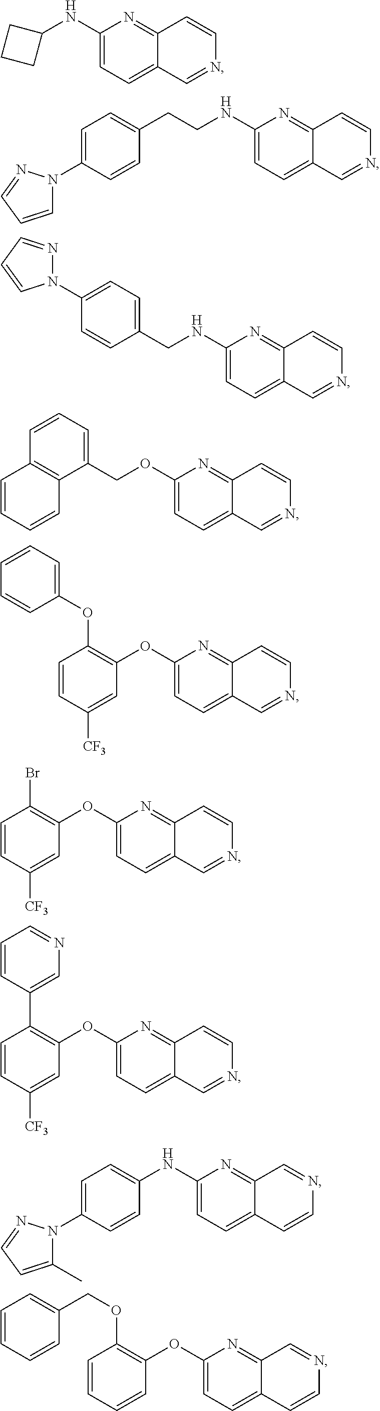 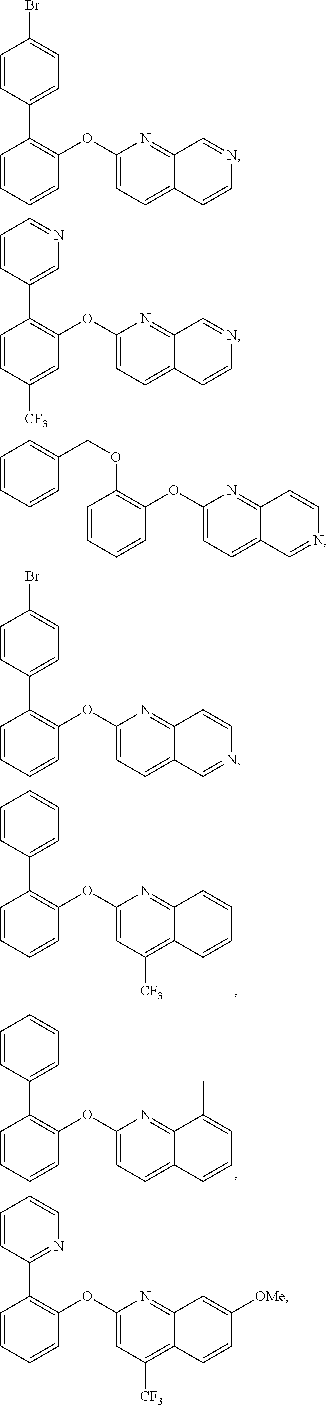 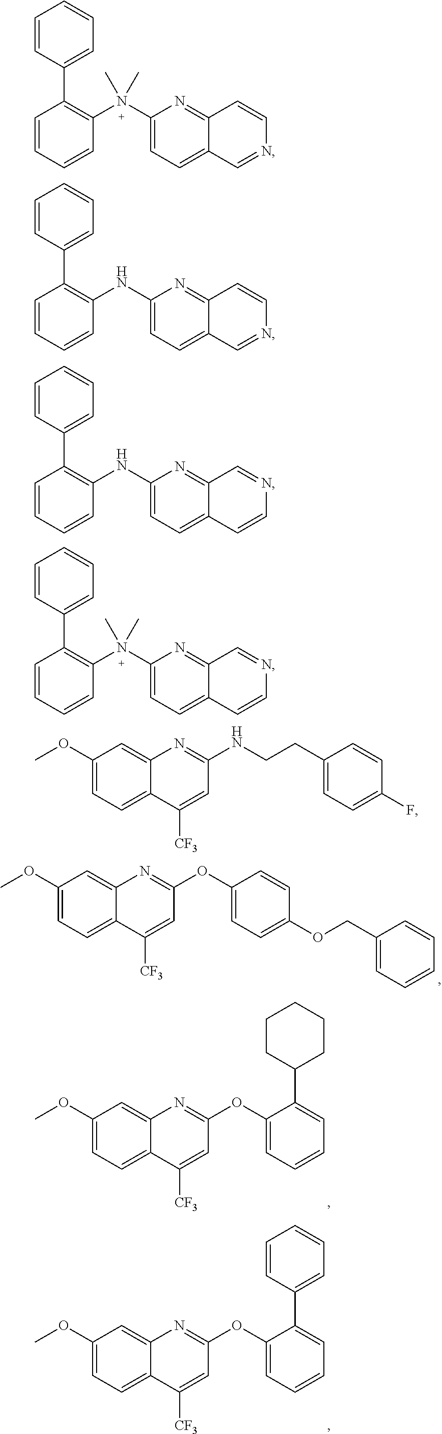 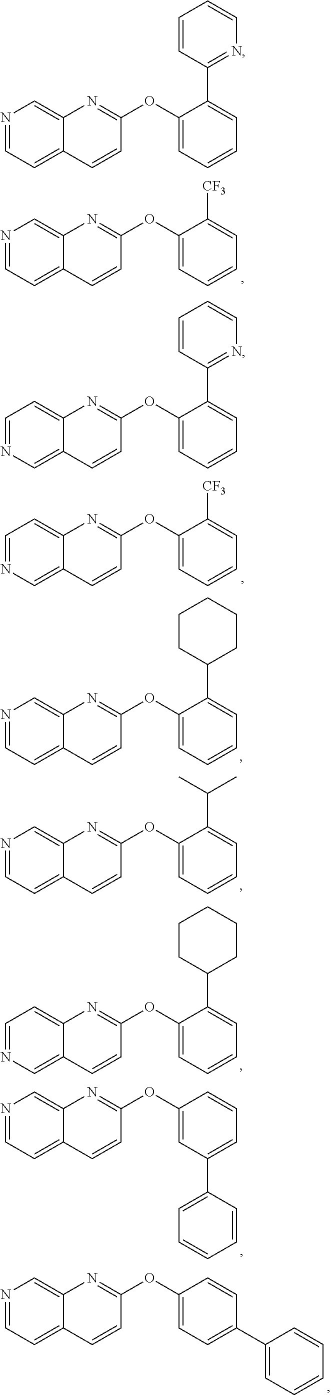 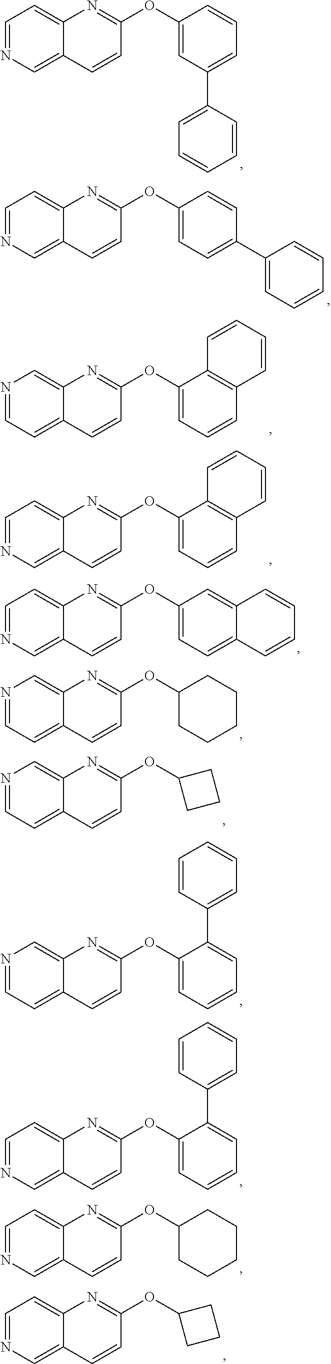 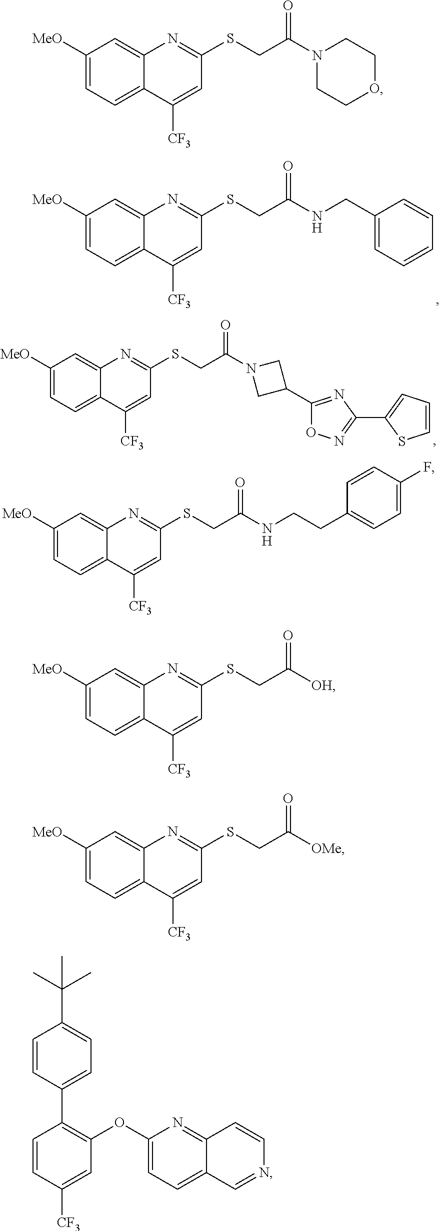 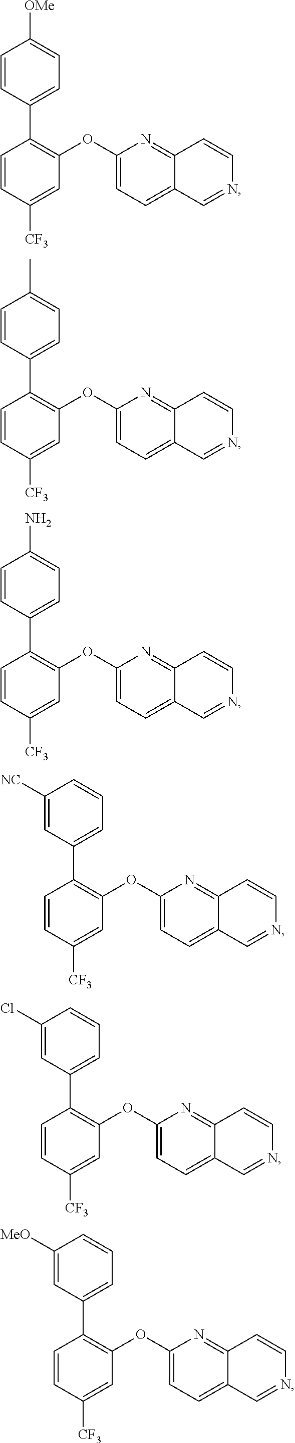 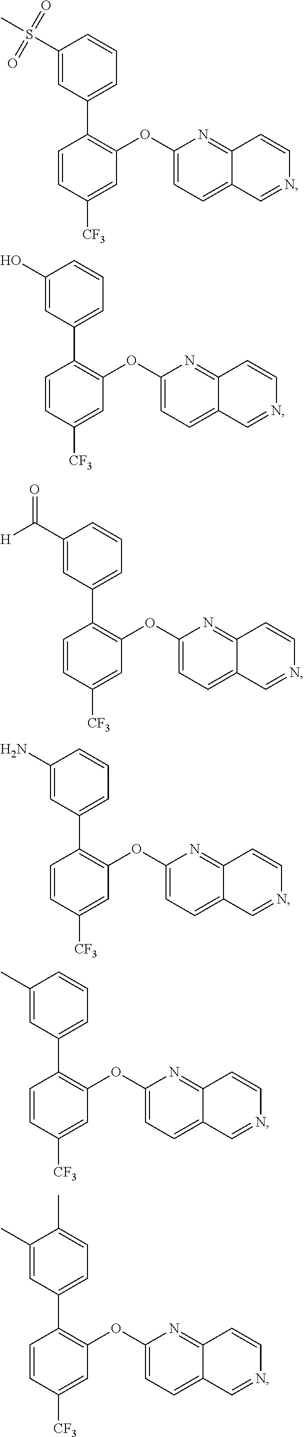 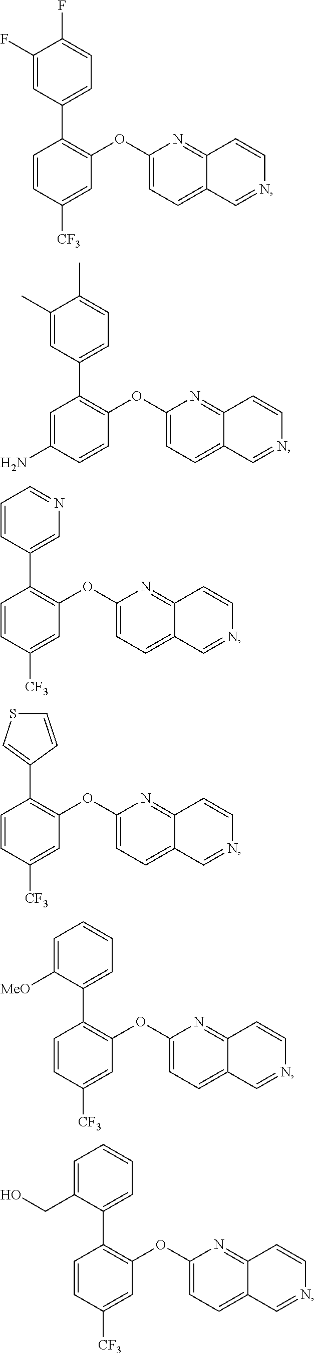 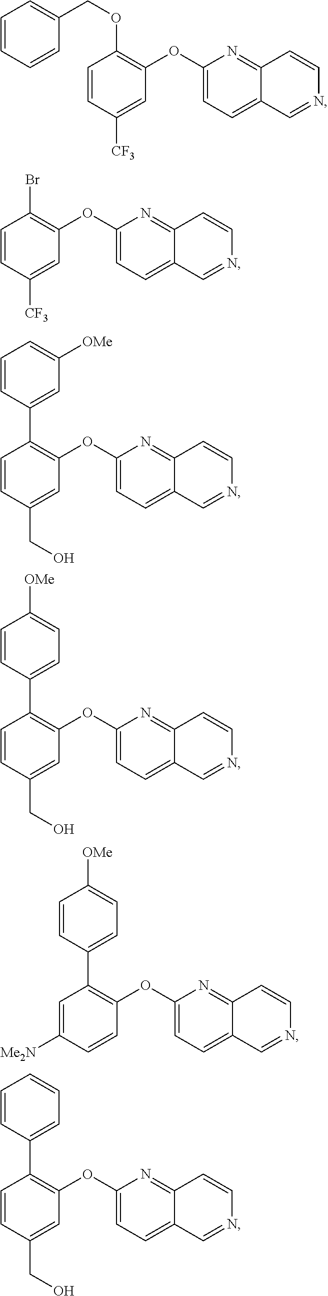 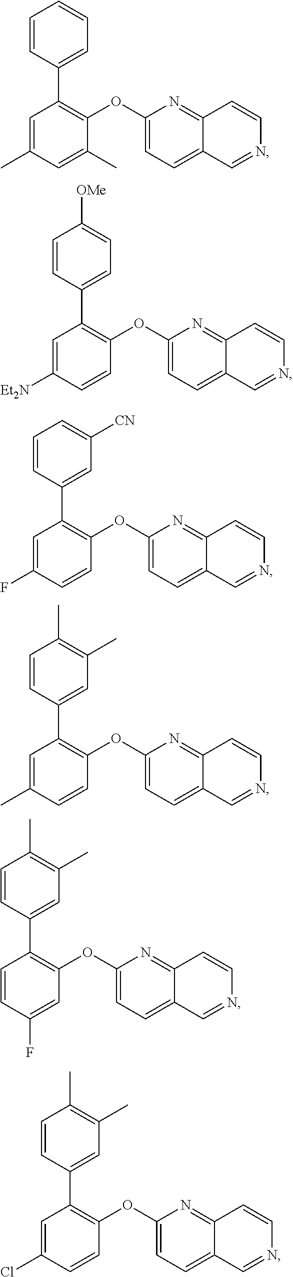 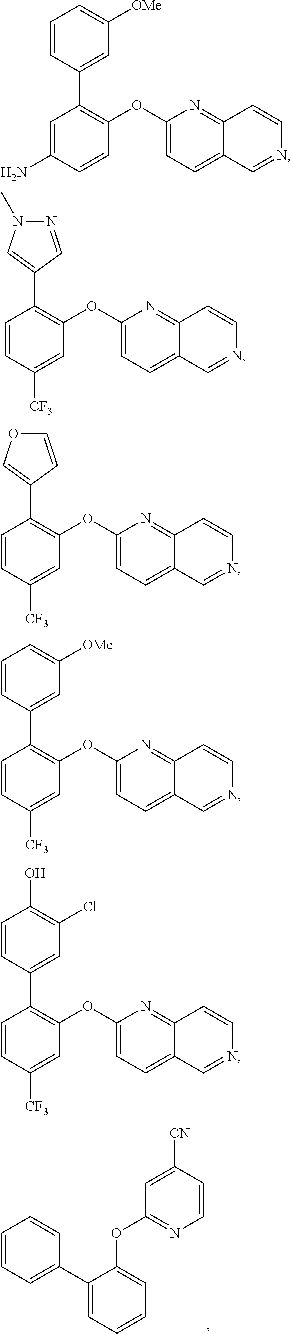 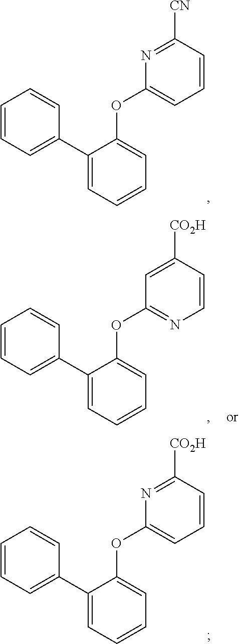
or a pharmaceutically acceptable salt thereof.

104.-105. (canceled)

106. A compound of the formula: 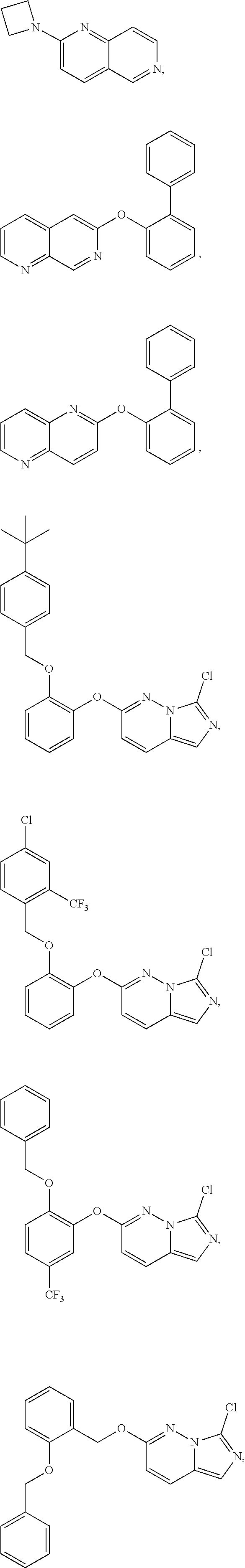 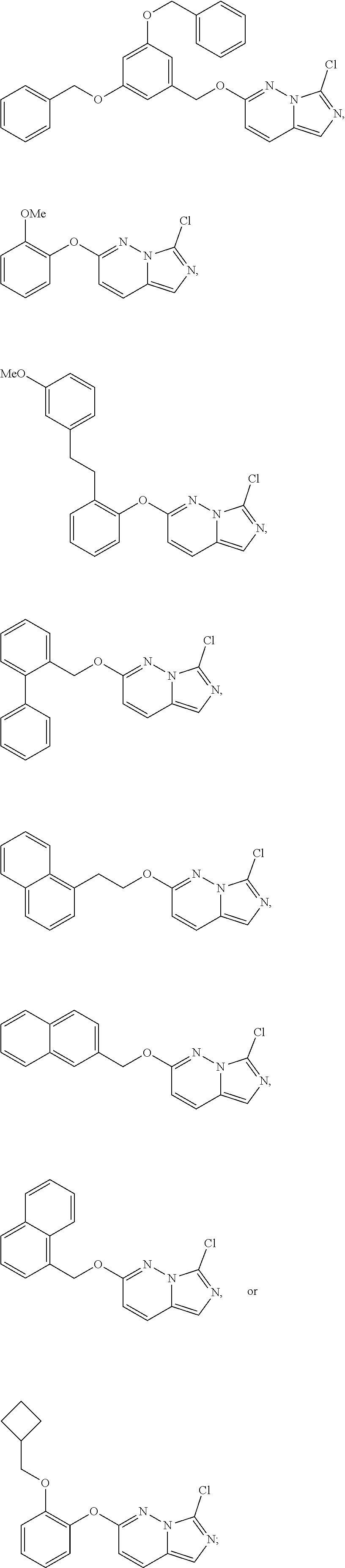
or a pharmaceutically acceptable salt thereof.

107. The compound of claim 27, selected from: 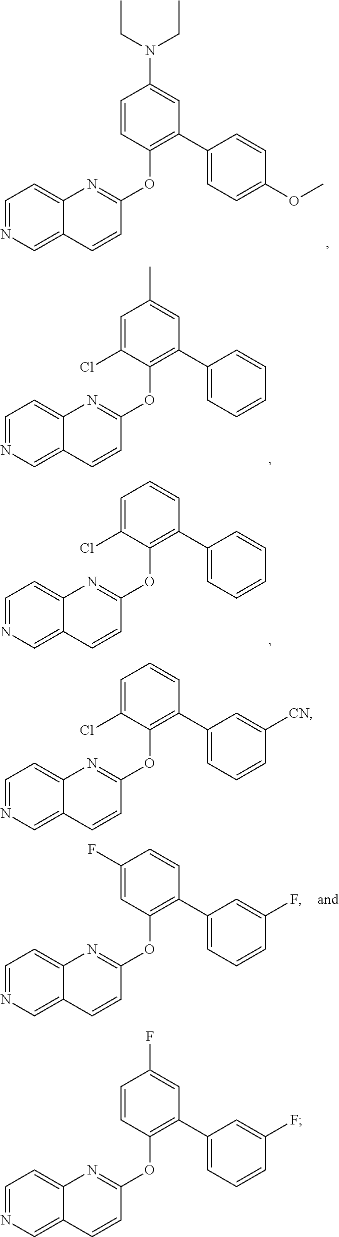
or a pharmaceutically acceptable salt thereof.

108. A pharmaceutical composition comprising:
(A) a compound according to claim 9; and
(B) an excipient, a pharmaceutically acceptable adjuvant, or a combination thereof.

109.-110. (canceled)

111. A method of treating a disease or disorder in a patient using a compound according to claim 9, comprising administering to the patient in need thereof a therapeutically effective amount of the compound;
optionally wherein:
the disease or disorder is selected from type 2 diabetes, obesity, heart disease, autoimmunity, chronic inflammation, neuroinflammation, anxiety, sepsis, sleep disorders, cancer, muscular dystrophy and cognitive disorders; or
the disease or disorder is a neurological disease, optionally wherein the neurological disease is an anxiety disorder; or
the disease or disorder is an autoimmune disorder; or
the disease or disorder is a muscular disorder, optionally wherein the muscular disorder is sarcopenia.

112.-116. (canceled)

117. The method according to claim 111, wherein
the method further comprises administering a second therapeutic agent.

118.-124. (canceled)
